(19) United States
(12) Patent Application Publication
(10) Pub. No.: US 2025/0043285 A1
(43) Pub. Date: Feb. 6, 2025

(54) Mammalian ATG8 Proteins and ATG9A Direct Sealing of Autophagosomal Membranes

(71) Applicant: UNM Rainforest Innovations, Albuquerque, NM (US)

(72) Inventors: Vojo Deretic, Placitas, NM (US); Graham Timmins, Albuquerque, NM (US); Rujeena Javed, Albuquerque, NM (US)

(21) Appl. No.: 18/761,527

(22) Filed: Jul. 2, 2024

Related U.S. Application Data
(60) Provisional application No. 63/524,711, filed on Jul. 3, 2023.

(51) Int. Cl. C12N 15/113; A61K 31/357; A61K 31/7034; A61K 38/10; A61K 45/06; A61P 3/00

(52) U.S. Cl. CPC C12N 15/113; A61K 31/357; A61K 31/7034; A61K 38/10; A61K 45/06; A61P 3/00; C12N 2310/141

(57) ABSTRACT

The present invention relates to discovery that modulators of ATG8 and/or ATG9A act in sequence to orchestrate the sealing of autophagosomal membranes. The present invention is directed to use of these agents and this mechanism in the treatment of various disease states and/or conditions, especially cancer.

### RELATED APPLICATIONS AND GRANT SUPPORT

This application claims the benefit of priority of United States provisional application number Ser. No. 63/524,711, filed Jul. 3, 2023, the entire contents of which application is incorporated by reference herein.

This invention was made with government support under grant nos. R37AI042999, R01AI111935 and center grant no. P20GM121176, awarded by the National Institute of Health. The government has certain rights in the invention.

### FIELD OF THE INVENTION

The present invention relates to discovery that modulators of ATG8 and/or ATG9A are useful in the treatment of diverse disease states and/or conditions which are related to autophagy mediation. The present invention is directed to this discovery and the use of modulators of ATG8 and/or ATG9A alone or in combination with additional bioactive agents can be used to treat, reduce the likelihood of, inhibit or ameliorate autophagy in the treatment of disease, especially the treatment of neurodegenerative diseases, infections, autoimmune diseases, inflammatory diseases and cancers. Methods of treating these disease states and/or conditions are disclosed using modulators of ATG8 and/or ATG9A (e.g. inhibitors and/or agonists) to patients with a disease state and/or condition including cancer and into tissues for the treatment of a disease state and/or condition including the inhibition, amelioration, reduction in the disease state and/or condition including metastasis and in recurrence of cancer in remission.

### SEQUENCE LISTING

This application incorporates by reference the XML file named “Sequence Listing N12-370US”, the date of creation of the XML file is Oct. 2, 2024, and the size of the XML file is 4,628 bytes.

### BACKGROUND AND OVERVIEW OF THE INVENTION

Autophagy represents a collection of diverse pathways sequestering cytoplasmic/cytosolic cargo for removal or recycling through degradation. Canonical autophagy in mammalian cells plays multiple roles in cellular metabolism, cytoplasmic quality control, anti-inflammatory processes and has been implicated in numerous fundamental and disease-related processes.

Canonical autophagy progresses through sequential stages including cytoplasmic emergence of double membrane organelles termed autophagosomes. The process of mammalian autophagosomal formation engages membrane sources from ER and endosomes. Other parts of the early secretory pathway are involved including ERGolgi intermediate compartment culminating in fusion of such membranes to form prophagophores. Prophagophores progress into phagophores by being decorated with mammalian ATG8 proteins (mATG8s). One of these, LC3B, represents a widely used autophagy marker. Phagophores expand via the action of multiple factors including lipid transfer proteins and lipid scramblases including ATG9A. The phagophores eventually close into a double membrane autophagosome to sequester the cargo. Whereas in yeast, autophagosomes fuse with the vacuole, mammalian autophagosomes continue to interact and fuse with the organelles and intermediates of the endosomal system. Multivesicular bodies endosomes (MVB) containing intraluminal vesicles are a typical fusion partner for autophagosomes to form amphisomes. These autophagic intermediates further mature acquiring lysosomal characteristics and progress into degradative autolysosomes.

The final steps of the autophagosome completion are believed to involve phagophore closure through a scission process catalyzed by ESCRTs. Two of the main ESCRTs identified in the process of autophagosomal closure are VPS37A and CHMP2A. How these proteins are recruited to the expanding phagophores to seal the autophagic membranes, if this occurs at a single point or multiple locations, and whether the sealed autophagosomes themselves are subject to homeostasis and their membranes need to be actively maintained, is not known.

One of the hallmarks of autophagy is the lipidation 11, 46 of mATG8s 47-49. There are two classes 49 of mATG8s: LC3s (LC3A, LC3B, LC3C) and GABARAPs (GABARAP, GABARAPL1, and GABARAPL2). The mATG8s′ lipidation cascade first delineated with yeast Atg8 50, 51 results in atg8ylation 3 of prophagophores. Whereas LC3B is commonly used to identify autophagosomes 31 it can be present on single membranes other than autophagosomes 52-62. The best understood function of mATG8s is the enhancement of cargo sequestration into autophagosomes 63-65 with additional functions proposed in membrane remodeling 66, membrane perturbation 67 during autophagosome biogenesis, autophagosome-lysosome fusion 49, and in membrane stress responses 62.

Autophagosomes can nevertheless form in cells lacking all principal mATG8s 68, 69 or in cells defective for mATG8 lipidation 70, albeit their size 69 or composition 70 and possibly quality are affected.

Recently, an assay directly measuring autophagosomal closure in cells has been developed 41, 42, 45 using HaloTag-LC3B (HT-LC3B) and fluorescent ligands that allows differentiating autophagosomes that are closed, based on their being impermeant to small probes, from those that are unclosed and so permeant to such probes. Using this system and a novel in vitro assay developed here, we report that mATG8s play a key role in directing specific ESCRT proteins to autophagosomes in order to seal them and maintain their membranes in an impermeant state. We furthermore reveal that ATG9A contributes to a key finishing step in these processes.

The canonical autophagy pathway in mammalian cells sequesters diverse cytoplasmic cargo within the double membrane autophagosomes that eventually convert into degradative compartments via fusion with endolysosomal intermediates. Here, the inventors report the discovery of the porousness of autophagosomal membranes appearing morphologically closed but being unable to mature into autolysosomes unless maintained in a sealed state. Using a combination of methods including a novel in vitro assay, the inventors uncovered a previously unappreciated function of mammalian ATG8 (mATG8) and ATG9A proteins which act in sequence to orchestrate the sealing of autophagosomal membranes. The mATG8 proteins GABARAP and LC3A bind to and recruit the key ESCRT-I components whereas ATG9A partners with ESCRT-III. The autophagic organelles in cells lacking mATG8s, ATG9A, or ESCRTs are permeant to small molecules, are arrested as amphisomes, and do not progress to functional autolysosomes. Thus, even when morphologically closed, autophagosomal organelles need to be maintained in a sealed state in order to become lytic autolysosomes. The invention relates to molecules which can promote these mechanisms in the treatment of disease states and conditions including inflammatory diseases and infectious diseases. Combinations of these agents can exhibit synergistic activity in the treatment of disease states and/or conditions.

### BRIEF DESCRIPTION OF THE INVENTION

The present invention relates to the discovery that Atg8 and/or Atg9A are related to autophagosomal closure in cells and that this mechanism is useful in the treatment of diverse disease states and/or conditions including neurodegenerative diseases, inflammatory disease, infectious disease, autoimmune disease and cancer, among others. In embodiments, the modulators of Atg8 and/or Atg9A (as inhibitors or agonists) may be combined with at least one additional autophagy modulator as describe herein, preferably an autophagy inhibitor, for example, tetrachlorisophthalonitrile, phenylmercuric acetate, 3-Methyladenine, wortmaninn, LY294002, cycloheximide, bafilomycin A1, hydroxychloroquine, Lys05, leupeptin, E64d, Pepstatin A, or a pharmaceutically acceptable salt thereof, among others as described herein and/or at least one additional anticancer agent as described herein.

In embodiments, the Atg8 modulator is an inhibitory tat-peptide for interference with VPS37A-GABARAP (mAtg8s) interaction. In embodiments, the inhibitory peptide of ATG8 is YGRKKRRQRRR-GG-MSWLFP (SEQ ID NO:1). This peptide is useful in the treatment of cancer and autoimmune diseases including rheumatoid arthritis, malaria, antiphospholipid antibody syndrome, lupus, antiphospholipid antibody syndrome, chronic urticaria and Sjogren's disease, among others wherein inhibition of autophagy is particularly impactful. In embodiments, the Atg8 inhibitory peptide of SEQ ID NO:1, above is used alone for the treatment of autophagy mediated diseases states or conditions or the inhibitory peptide may be combined with an effective amount at least one additional inhibitor selected from the group consisting of OleA (oleuropein or oleuropein aglycone), hsa-miR-34a (Accession Number NR_029610; NR_029610.1), preferably as has-miR-34a-5p (UGGCAGUGUCUUAGCUGGUUGU SEQ ID NO: 2) or has-miR-34a-3p (CAAUCAGCAAGUAUACUGCCCU SEQ ID NO:3) or AT110 inhibitor as depicted below:

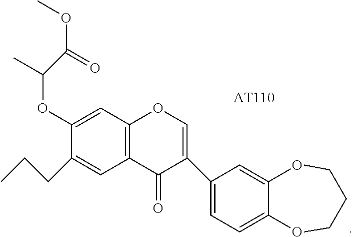 [SMILES: CCCc1cc2c(=O)c(-c3ccc4c(c3)OCCCO4)coc2cc1OC(C)C(=O)OC ; "AT110"]

In embodiments, the Atg8 inhibitory peptide (SEQ ID NO:1, above) is combined with an effective amount of an Atg9A inhibitory peptide (Tat-Site1 peptide: YGRKKRRQRRR-GG-WEGQLQDLVLDEY, SEQ ID NO:4) in order to enhance the effect of the Atg8 inhibitory peptide in treating autophagy mediated disease states and/or conditions. These disease states and/or conditions include cancer, including metastasis of cancer, or the inhibition, treatment and/or prevention of one or more disease states or conditions in which the inhibition of autophagy provides a favorable result including rheumatoid arthritis, malaria, antiphospholipid antibody syndrome, lupus, chronic urticaria and Sjogren's disease, among others.

In other embodiments, the Atg8 inhibitory peptide of SEQ ID NO: 1 may be combined with an effective amount of at least one autophagy modulator agonist as otherwise described herein in order to treat numerous autophagy mediated disease states and/or conditions as otherwise described. These modulators are disclosed in detail herein below.

In embodiments, the present invention is directed to methods of inhibiting autophagy in a biological system, in particular a patient or subject for the treatment of an autophagy mediated disease state and/or condition. In this aspect of the invention, a compound or combination of compounds as otherwise described herein is presented to the biological system, including administration to a patient or subject in need, in order to inhibit autophagy. The resulting inhibition may be monitored or applied in the biological system to effect a favorable result, including the inhibition, treatment and/or prevention of cancer, including metastasis of cancer, or the inhibition, treatment and/or prevention of one or more disease states or conditions in which the inhibition of autophagy provides a favorable result including rheumatoid arthritis, malaria, antiphospholipid antibody syndrome, lupus, chronic urticaria and Sjogren's disease, among others.

Methods of inhibiting, treating and/or reducing the likelihood of cancer, including metastasis of cancer and drug resistant cancer, comprises administering to a patient in need at least one compound according to the present invention, optionally in combination with at least one additional anticancer agent as otherwise described herein.

The present invention also relates to treating, inhibiting and/or preventing diseases, diseases states and/or conditions in a patient in need in which the inhibition of autophagy provides a favorable outcome, including cancer, rheumatoid arthritis, malaria, antiphospholipid antibody syndrome, lupus, chronic urticaria and Sjogren's disease, the method comprising administering to said patient at least one compound according to the present invention.

In an additional embodiment, the present invention is directed to pharmaceutical compositions which comprise an effective amount of a compound which is an Atg8 modulator, often an Atg8 inhibitor (e.g. an Atg8 inhibitor peptide according to SEQ ID NO:1) as described above, in combination with at least one autophagy modulator, and/or at least one additional bioactive agent as described herein in combination with a pharmaceutically acceptable carrier additive and/or excipient.

Additional embodiments of the present invention may be readily gleaned from the detailed description of the invention which follows, including the experiments which are presented herein below.

### BRIEF DESCRIPTION OF THE FIGURES

FIGS. 1A, 1B, 1C, 1D, 1E, 1F and 1G show that ATG8s play role in LC3B⁺ membrane sealing through ESCRT-I. 1A, GST pulldown analysis of in vitro translated and radiolabeled [³⁵S] Myc-VPS37A with GST or GST tagged LC3A, LC3B, LC3C, GABARAP, GABARAP-L1 and GABARAP-L2. 2% of input was loaded. 1B, AlphaFold predicted complexes between VPS37^{AdeltaCEV} and GABARAP. 1C, Schematic representation of HaloTag (HT)-LC3B MIL/MPL assay adapted for quantitative high content microscopy (HCM). MIL⁺ profiles, HT-LC3B accessible to membrane impermeant ligand; MPL⁺ profiles, HT-LC3B remaining available (not saturated with MIL) to membrane permeant ligand. 1D, MIL/MPL assay in HeLa^{WT} and HeLa^{HexaKO} expressing HT-LC3B, starved in EBSS for 90 min±100 nM BafA1. Cells were sequentially incubated with HT ligands MIL and MPL, and HCM quantification carried out; (d-i) MIL-accessible membrane-bound HT-LC3B; (d-ii) MPL-accessible membrane-bound HT-LC3B (remaining available to MPL after MIL saturation). (d-iii) MIL/MPL puncta ratio. Circles, control samples (full medium); rectangles (EBSS; triangles, EBSS+BafA1; green, MIL; red, MPL, gray, MIL/MPL ratios. Images, HCM images (masks: white, primary objects/cells; green, MIL profiles; red, MPL profiles), one of 60 fields/well, >500 primary objects (cells)/well; 6 wells per sample/plate, triplicate independent biological samples/plates. Statistical significance was determined by one-way ANOVA followed by Tukey's multiple comparison test, P<0.01. All values are mean±SD, n=3 biologically independent experiments, 1E-1G, Selected electron microscopy (EM) images of Hela^{WT} and Hexa^{KO} EBSS-induced for autophagy for 90 min and their quantification (graph in 1G). AP, autophagosomes (phagophores or double membrane autophagosomes with content of similar electron-density to surrounding cytosol); Amph, amphisomes; AL, autolysosomes; MVB, multivesicular bodies; LELy, late endosomes or lysosomes. Statistics, unpaired t-test, two groups: HeLa^{WT} (in 1E) and HeLa^{HexaKO} (in 1H) (93 images each; sample mean, SE). 1H, Super-resolution (dSTORM) of HeLa^{HexaKO} HT-LC3B stained with MIL (note staining of the interior of globular structures). Scale bars, 10 μm for HCM and 200 nm for EM images.

FIGS. 2A, 2B and 2C show an In vitro system for assaying membrane sealing (SolVit). 2A, Representation of SolVit (sealing of organellar limiting membranes in vitro). Left (general schematic), cells serving as sources of donor or acceptor PNS (post nuclear supernatant) are starved in EBSS for 90 min, extracts prepared via repeat passages through a needle, and PNS collected after centrifugation at 12,000 g. Upon incubation, reaction products are pelleted, resuspended and mounted in Prolong-Gold in 96-well plates for HCM. Right, Acceptor PNS, HeLa^{HexaKO} HT-LC3B (HeLa^{HexaKO} cells stably expressing HT-LC3B) was incubated with donor PNS (HeLa^{WT} or HeLa^{HexaKO})±ATP for 1 h, stained with MIL, fixed with 4% PFA, super-stained with MPL, and processed for HCM. 2B, 2C SolVit assay with HeLa^{WT} and HeLa^{HexaKO} as donors, HeLa^{HexaKO} HT-LC3B as acceptor. (2B), SolVit HCM images (example). MIL⁺ (green), unsealed LCB⁺ membranes; MPL⁺ (red), sealed LCB⁺ membranes; (2C), Quantification of MIL⁺ and MPL⁺ profiles (60 fields/well acquired): (c-i) MIL⁺ (green) puncta per field. (c-ii) MPL⁺ (red) puncta per field. (c-iii) MIL⁺/MPL⁺ profile ratios (gray). Statistical significance was determined by one-way ANOVA followed by Tukey's multiple comparison test, P<0.01. All values are mean±SD, n=3 biologically independent experiments, each HCM experiment: 1,000 valid primary objects/cells per well (max fields per well, 60), 5 wells/sample, Scale bar, 3 μm.

FIGS. 3A, 3B, 3C, 3D, 3E and 3F show that GABARAP and LC3 mATG8s subsets contribute to membrane sealing. 3A, Western blot, Hela WT, HeLa^{HexaKO}, HeLa^{LC3TKO}, HeLa^{GABATKO} stably expressing HT-LC3B. 3B, Cells were starved in EBSS for 90 min±100 nM BafA1, sequentially incubated with MIL (b-i; green, marker of unsealed membranes) and MPL (b-ii, red; marker of sealed membranes) and quantified by HCM; (b-iii) MIL⁺/MPL⁺ puncta ratio (gray), 3C, Schematics of In vitro complementation in the SolVit system: (c-α) HeLa^{LC3TKO}-HT-LC3B PNS (acceptor) with HeLa^{WT}, Hela^{GABATKO} or HeLa^{LC3TKO} PNS (donors); (c-β) HeLa^{GABATKO}-HT-LC3B PNS (acceptor) with HeLa^{WT}, Hela^{GABATKO} or HeLa^{LC3TKO} PNS (donors). 3D, HCM images, example from SolVit complementation experiments. Green puncta, MIL⁺LC3B unsealed membranes, red, MPL⁺ sealed membranes. 3E, Complementation analysis (quantification) of HeLa^{LC3TKO}-HT-LC3B PNS with PNS from HeLa^{WT}, HeLa^{LC3TKO} or Hela^{GABATKO} cells±ATP, 1 h, 37° C. 3F, Complementation analysis (quantification) of HeLa^{GABATKO}-HT-LC3B PNS with PNS from HeLa WT, HeLa^{LC3TKO} and Hela^{GABATKO} cells±ATP, 1 h, 37° C. Statistical significance was determined by one-way ANOVA followed by Tukey's multiple comparison test, P<0.01. All values are mean±SD, n=3 biologically independent experiments, each HCM experiment: 1,000 valid primary objects/well, 5 wells/sample, Scale bars represent 3 μm.

FIGS. 4A, 4B, 4C, 4D and 4E show that ATG9A contributes to membrane sealing. 4A, Western blot analysis of Huh7^{WT} HT-LC3B and Huh7^{ATG9AKO} HT-LC3B. 4B, HT-LC3B MIL/MPL quantification by HCM in Huh7 HT-LC3B stable ATG9A WT and ATG9A^{KO}. Huh7 ATG9A^{WT} or ATG9A^{KO} cells stably expressing HT-LC3B were treated with 20 μM CCCP±100 nM BafA1 for 6 h. (b-i) MIL⁺ puncta (green). (b-ii) MPL⁺ puncta (red). (b-iii) MIL/MPL ratio (gray). 4C, HCM images: green mask, MIL⁺ (unsealed membranes); red mask, MPL⁺ (sealed membranes). White masks, algorithm-defined cell boundaries. 4D, Huh7 ATG9A^{WT} and ATG9A^{KO} cells stably expressing HT-LC3B were transfected with FLAG-ATG9A^{WT} or FLAG-ATG9A^{M33}; empty, FLAG vector only. Graph, MIL⁺ puncta/cell (green). 4E, HCM representative images corresponding to d. Statistical significance was determined by one-way ANOVA followed by Tukey's multiple comparison test, P<0.01. All values are mean±SD, n=3 biologically independent experiments, each HCM experiment: 1,000 valid primary objects/cells per well, 6 wells/sample, scale bar represents 10 μm.

FIGS. 5A, 5B and 5C show ATG9A effects on sealing of LC3⁺ membranes in vitro. 5A, Schematic representation SolVit complementation assay with ATG9A. 5B, HCM example images of SolVit assay incubation products; Huh7^{ATG9AKO} HT-LC3B, Huh7^{WT} and Huh7^{ATG9AKO} cells were treated with 20 μM CCCP for 6 h. Green profiles (MIL⁺ unsealed LCB⁺ membranes); red profiles (MPL⁺ sealed LCB⁺ membranes). 5C, Quantification of SolVit MIL⁺ and MPL⁺ profiles. Huh7^{ATG9AKO} HT-LC3B PNS was incubated with Huh7^{WT} or Huh7^{ATG9AKO} PNS±ATP for 1 h. Reaction products were stained with MIL and MPL sequentially. (c-i) MIL⁺ profiles (green). (c-ii) MPL⁺ profiles (red). (c-iii) MIL/MPL ratio (gray). Statistical significance was determined by one-way ANOVA followed by Tukey's multiple comparison test, P<0.01. All values are mean±SD, n=3 biologically independent experiments, each HCM experiment: 1,000 valid primary objects/cells per well, 5 wells/sample, Scale bars represent 3 μm.

FIGS. 6A, 6B, 6C, 6D, 6E, 6F and 6G show that ATG9A and its partners contribute to sealing of LC3B membranes. 6A, MIL/MPL assay quantification of IQGAP1 Knockdown in Huh7 stably expressing HT-LC3B. Huh7 siScr and Huh7 siIQGAP1 in cells expressing HT-LC3B were treated with 20 μm CCCP for 6 h±100 nM BafA1. Cells were sequentially incubated with (a-i) membrane-impermeable HT ligand (MIL) to stain membrane-bound HT-LC3B-II that is accessible to the cytosol and (a-ii) membrane-permeant HT ligand (MPL) to stain LC3B-II that is sequestered within sealed membrane, and puncta quantified in ii,iii; (a-iii) MIL/MPL puncta ratio. 6B, Western blot analysis of IQGAP1 knockdown in HEK293T. 6C, Co-IP analysis of CHMP2A and GFP-ATG9A after knockdown IQGAP1. 6D, Quantification of CHMP2A ratio to GFP-ATG9A after knockdown IQGAP1 after CCCP treatment. 6E, MIL/MPL assay quantification of CHMP2A Knockdown in Huh7 stably expressing HT-LC3B. Huh7 siScr and Huh7 siCHMP2A in cells expressing HT-LC3B were treated with 20 μm CCCP for 6 h in the presence or absence of 100 nM BafA1. Cells were sequentially incubated with (e-i) membrane-impermeable HT ligand (MIL) to stain membrane-bound HT-LC3B-II that is accessible to the cytosol and (e-ii) membrane-permeant HT ligand (MPL) to stain LC3B-II that is sequestered within sealed membrane and quantified (e-iii) MIL/MPL puncta ratio. 6F, Quantification of CHMP4B puncta in CCCP or DMSO treated Huh7WT and ATG9AKO. 6G, HCM images, examples corresponding to f. Statistical significance was determined by one-way ANOVA followed by Tukey's multiple comparison test, P<0.01. All values are mean±SD, n=3 biologically independent experiments, each HCM experiment: 1,000 valid primary objects/cells per well, 6 wells/sample.

FIGS. 7A, 7B, 7C, 7D, 7E, 7F, 7G, and 7H show that ATG8s interact with ESCRT-I component VPS37A. 7A, Co-IP analysis of interactions between FLAG-VPS37A and GFP-LC3A and GFP-GABARAP (GABA) in HEK293T cells. 5% of input was loaded. 7B, Schematic of VPS37A full length and deletion constructs: (i) VPS37A^{FL}, full length, (ii) VPS37A^{deltaUEV} deletion of UEV (cyan), (iii) UEV^{VPS37A}, UEV domain only (cyan), (iv) VPS37A^{deltaN(1-90)} deletion of residues 1-90, (v) VPS37A^{deltaN(1-20)}, deletion of residues 1-20 (red), (vi) VPS37A^{Nter-EGFP}, N-ter 1-22 chimera with GFP. 7C, GST-Pull down of Myc-UEV^{VP37A} and Myc-VPS37AdeltaCEV with GST or GST-LC3A, GST-LC3C and GST-GABARAP, 2% of Input was loaded. 7D, Co-IP between GFP-LC3A with FLAG-VPS37A^{FL}, UEV^{VPS37A} and VPS37A^{deltaCEV}. 7E, VPS37AdeltaCEV AlphaFold predicted structure; sequence, 1-22 residues, N-terminus of VPS37A (red, residues predicted to contact mATG8s. 7F, Representation of AlphaFold predicted complexes (rank 1; VPS37A^{deltaCEV} and GABARAP). 7G,7H Co-IP analyses in HEK293T cells expressing FLAG-VPS37A and GFP-LC3A (in 7H) or GFP-GABARAP (in 7G).

FIGS. 8A, 8B, 8C and 8D indicates that AlphaFold prediction showed that VPS37A^{deltaUEV} interacts with mATG8s. 8A,8B The predicted modeled were re-ranked according to the predicted Local Distance Difference Test score (pLDDT) for each model (LCSA in 8A, GABARAP in 8B). ColabFold IDDT graph shows all five ranked models and their predicted IDDT Ca score at each residue. 8C, 8D prediction frequencies in 5 simulations of LC3A and GABARAP were plotted first. 8C, first five long bars of the plot represents VPS37A^{N-ter} residues interaction with LC3A 8D, first six long bars of the plot represents VPS37A^{N-ter} residues interaction with GABARAP. 8E, rank 1 model predicted structure showing a sidechain packing albeit with differences within the hydrophobic pocket of GABARAP and LC3A when compared to the known structure of LC3C. Cyan color represents PLEKHM1 LIR peptide, green color represents VPS37A^{N-ter}.

FIGS. 9A, 9B, 9C and 9D show the Ultrastructural analysis of organelles in HeLa^{WT} and HeLa^{HexaKO} cells. 9A, Electron microscopy (EM) images of autophagic organelles in cells induced for autophagy by starvation in EBSS for 90 min (see FIG. 2). Blue arrows, autophagosomal structures (autophagosomes and phagophores). Blue asterisks, amphisomes. 9A, HeLa^{WT} (a-i-iii); 9B, HeLa^{HexaKO}. 9C, MVB endosomes (no differences noted between HeLa^{WT} HeLa^{HexaKO} cells) 9D. Lysosomal structures: ALys, autolysosomes (HeLa^{WT}). Lys, lysosomes. Electron-dense dots, glycogen granules. Scale bar, 200 nm.

FIG. 10 shows super-resolution microscopy (dSTORM) which reveals a spectrum of permeant membranous structures in cells devoid of principal mATG8s. HeLa^{HexaKO} cells stably expressing HT-LC3B were starved in EBSS for 90 min to induce autophagy. After selective permeabilization of plasma membrane, cells were then stained with membrane-impermeable HT ligand (MIL; Alexa fluor 660) and processed for super-resolution microscopy (dSTORM; see methods). White rectangles, zoomed insets. Cyan arrowheads, crescent structures (phagophores). Magenta arrowheads, globular structures with internal membranes accessible to the MIL probe. Scale bar, 1 μm.

FIG. 11 shows super-resolution microscopy (dSTORM) which reveals a spectrum of permeant membranous structures in cells devoid of principal mATG8s. HeLa^{HexaKO} cells stably expressing HT-LC3B were starved in EBSS for 90 min to induce autophagy. After selective permeabilization of plasma membrane, cells were then stained with membrane-impermeable HT ligand (MIL; Alexa fluor 660) and processed for super-resolution microscopy (dSTORM; see methods). White rectangles, zoomed insets. Cyan arrowheads, crescent structures (phagophores). Magenta arrowheads, globular structures with internal membranes accessible to the MIL probe. Scale bar, 1 μm.

FIGS. 12A, 12B, 12C, and 12D show that both GABARAP and LC3 subsets of mATG8s maintain the integrity of LC3B⁺ membranes. 12A. HeLa^{WT}, HeLa^{HexaKO}, HeLa^{LC3TKO} and HeLa^{GABATKO} stably expressing HT-LC3B were starved to induced autophagy in EBSS 90 min±100 nM BafA1. MIL/MPL assay HCM images representing MIL⁺ (green) LC3B⁺ unsealed membrane, MPL⁺ (red) LC3B⁺ sealed membrane (LC3B sequestered within the sealed membranes). 12B, HeLa^{LC3TKO} expressing HT-LC3B cells complemented with LC3A. Cells were transfected with GFP or GFP-LC3A, plasma membrane selectively permeabilized, and endomembranes stained with MIL. HCM images, GFP or GFP-LC3A transfected cells (red pseudocolor). MIL⁺ puncta were counted and quantified. Graph, MIL⁺ puncta/cell in GFP or GFP-LC3A transfected cells (identified by gating). 12C, HeLa^{GABATKO} expressing HT-LC3B complemented with GFP-GABARAP. Cells were transfected with GFP or GFP-GABARAP. HCM images, GFP or GFP-GABARAP transfected cells stained with MIL (red pseudocolor). Graph quantification of MIL⁺ puncta/cell in GFP or GFP-GABARAP transfected cells. 12D, HeLa^{WT} and HeLa^{HexaKO} cells were transfected with LC3BHibit plasmid. Cells were induced to autophagy with mTOR inhibitor PP242 (2 μM) for 6 h. Plasma membrane was selectively permeabilized with PMP (4 nM). Bioluminescence was measured in a luminometer plate reader. Circles, control; squares, PP242-treated cells. Statistical significance was determined by one-way ANOVA followed by Tukey's multiple comparison test, P<0.01. All values are mean±SD, n=3 biologically independent experiments, each HCM experiment: 1,000 valid primary objects/cells per well, 6 wells/sample, Scale bars, 10 μm.

FIGS. 13A, 13B, 13C, 13D, 13E, 13F, 13G, 13H, 13I, 13J and 13K show that ATG9A interacts with ESCRTs and is required for membrane sealing during mitophagy. 13A. Schematic representation of proximity biotinylation proteomics (process stages) for LC MS/MS identification of APEX2-ATG9A partners in cells subjected to EBSS-induced autophagy and CCCP-induced mitophagy. 13B, List of ATG9A's key ESCRTs interactors and their peptide counts. 13C, HeLa^{WT} and HeLa^{ATG9AKO} cell line stably expressing YFP-Parkin were induced for mitophagy by CCCP or Olygomycin A and Antimycin A (OA). HCM was used to quantify mitophagy by determining remaining mtDNA puncta per cell. Blue squares, HeLa^{WT} stably expressing YFP-Parkin; red squares, HeLa^{ATG9AKO} stably expressing YFP-Parkin. 13D, HCM images illustrating mtDNA (red mask) in HeLa^{WT} and HeLa^{ATG9AKO} cells stably expressing YFP-Parkin. 13E, Western blot analysis of Huh7^{WT} and Huh7^{ATG9AKO} cells, under control conditions (full medium) or subjected to CCCP-induced mitophagy. 13F, Western blot analysis of Huh7^{WT} and Huh7^{ATG9AKO} in cells under CCCP-induced mitophagy conditions in presence or absence of BafA1 (100 nM); immunoblot indicates accumulation of Parkin and COX-II in the presence of BafA and Huh7^{ATG9AKO}. 13G, Western blot analysis of LC3B lipidation in control and CCCP-induced mitophagy condition in Huh7^{ATG9AWT} or Huh7^{ATG9AKO}. 13H, Quantification of LC3B dots in Huh7^{WT} and Huh7^{ATG9AKO} cells under control conditions or subjected to CCCP-induced mitophagy. 13I, Western blot analysis of protease protection assay of p62 and NDP52 in Huh7^{WT} or Huh7^{ATG9AKO} extracts±proteinase K (50 μg) and TritonX-100 (1%). 13J,13K Quantification of p62 and NDP52 levels in control and proteinase K-treated samples. Statistical significance was determined by one-way ANOVA followed by Tukey's multiple comparison test, P<0.01. All values are mean±SD, n=3 biologically independent experiments, each HCM experiment: 1,000 valid primary objects/cells per well, 6 wells/sample. Scale bars, 10 μm.

FIGS. 14A, 14B, 14C, 14D, 14E, 14F, 14g, and 14H show that ATG9A ESCRT interactors are required for LC3B⁺ membrane sealing. 14A. Western blot of Huh7^{WT} and VPS37A^{KO} 14B, Quantification graphs of MIL/MPL assay of Huh7^{WT} and Huh7^{VPS37AKO} stably expressing HT-LC3B. (b-i) MIL to stain membrane-bound HT-LC3B-II that is accessible to the cytosol and (b-ii) membrane-permeant HT ligand (MPL) to stain LC3B-II that is sequestered within sealed membrane. (b-iii) MIL/MPL puncta ratio. 14C, HCM images representing MIL⁺ unsealed membrane after induction mitophagy by CCCP (20 μM) in ±100 nM BafA1. MPL⁺ sealed membrane in Huh7^{WT} and Huh7^{VPS37AKO} cells stably expressing HT-LC3B. 14D, Western blot of Huh7^{WT} and VPS37B^{KO} 14E, Quantification graphs of MIL/MPL assay of Huh7^{WT} and Huh7^{VPS37BKO} stably expressing HT-LC3B in CCCP-induced mitophagy in ±BafA1 (100 nM). (e-i) MIL/MPL assay for quantifying MIL⁺ unsealed membrane and (e-ii) MPL⁺ sealed membrane in Huh7^{WT} and Huh7^{VPS37AKO} HT-LC3B, (c-iii) MIL/MPL puncta ratio 14F, HCM images representing MIL⁺ unsealed membrane and MPL⁺ sealed membrane after induction of mitophagy by CCCP (20 μM) in ±BafA1 (100 nM) in Huh7^{WT} and Huh7^{VPS37BKO} cells stably expressing HT-LC3B cells. 14G, Huh7^{VPS37AWT} and VPS37A^{KO} cells stably expressing HT-LC3B were complemented with VPS37A constructs. MIL⁺ puncta (green) were quantified, representing unsealed LC3B⁺ membranes. CCCP for 6 h. Statistical significance was determined by one-way ANOVA followed by Tukey's multiple comparison test, P<0.01. All values are mean±SD, n=3 biologically independent experiments, each HCM experiment: 1,000 valid primary objects/cells per well, 6 wells/sample, Scale bars, 10 μm. 14H, HCM images of Huh7^{VPS37AKO} HT-LC3B with VPS37A constructs (VPS37A^{Full}, FLAG-VPS37A^{deltaN(1-90)}, FLAG-VPS37A^{deltaN(1-20)} and FLAG-VPS37A^{Nter-GFP}). Images representing FLAG-Tag VPS37A constructs in pseudo color red and MIL⁺ puncta in pseudo color green.

FIGS. 15A, 15B, and 15C show that ATG9A partner with ESCRT-I protein VPS37A, to maintain membrane integrity. 15A, Schematic representation SolVit system for Huh7^{VPS37KO} HT-LC3B. Huh7^{VPS37KO} HT-LC3B cells were treated with CCCP (20 μM) for 6 h and Post Nuclear Supernatant (PNS) was collected by centrifugation at 12,000 g. PNS of Huh7^{VPS37KO} HT-LC3B stable expressing HT-LC3B was incubated in vitro with Huh7^{WT} and Huh7^{VPS37AKO} PNS in presence and absence of ATP for 1 h. PNS mix were stained with MIL to label membrane-bound HT-LC3-II and fixed with 4% PFA, sequentially samples were treated with MPL to stain LC3-II that is sequestered within the membranes. 15B, HCM images representation of SolVit. (b-i) MIL⁺ (green) represents unsealed LCB⁺ membrane and (b-ii) MPL⁺ (red) represents sealed LCB⁺ membrane (b-iii). MIL/MPL ration shown in gray color. 15C, HCM images one of 60 fields depicting MIL⁺ unsealed LC3B⁺ membranes and MPL⁺ sealed membranes in presence and absence of ATP. Statistical significance was determined by one-way ANOVA followed by Tukey's multiple comparison test, P<0.01. All values are mean±SD, n=3 biologically independent experiments, each HCM experiment: 1,000 valid primary objects/cells per well, 6 wells/sample, Scale bars, 3 μm.

FIGS. 16A, 16B, 16C, 16D, 16E and 16F show that ATG9A partners with ESCRT interactors to maintain membrane integrity. 16A, Western blot analysis of IQGAP1 knockdown in Huh7 HT-LC3B cells. 16B, HCM images representing MIL⁺ (green mask) and MPL⁺ (red mask) puncta in siRNA control and siRNA-IQGAP1 knockdown Huh7 HT-LC3B cells after induction of mitophagy and sequential staining of MIL and MPL in the presence of plasma membrane permeabilizer. 16C, Western blot analysis of CHMP2A knockdown in Huh7 HT-LC3B cells. 16D, HCM images represents MIL⁺ (green mask) and MPL⁺ (red mask) puncta in siRNA control and siRNA-CHMP2A knockdown Huh7 HT-LC3B cells after induction of mitophagy. 16E, Quantification of overlap area between CHMP4B and LC3B in Huh7WT and ATG9A^{KO}. 16F, HCM images representing yellow puncta as overlap area between CHMP4B (green) and LC3B (red) in Huh7WT and ATG9A^{KO}. Statistical significance was determined by one-way ANOVA followed by Tukey's multiple comparison test, P<0.01. All values are mean±SD, n=3 biologically independent experiments, 6 wells/sample. Scale bars, 10 μm.

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

The term “compound” or “agent”, as used herein, unless otherwise indicated, refers to any specific chemical compound disclosed herein and includes tautomers, regioisomers, geometric isomers as applicable, and also where applicable, optical isomers (e.g. enantiomers) thereof, as well as pharmaceutically acceptable salts thereof, including in the case of peptides disclosed herein. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

The term “patient” or “subject” is used throughout the specification within context to describe an animal, generally a mammal, including a domesticated mammal including a farm animal (dog, cat, horse, cow, pig, sheep, goat, etc.) and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human.

The terms “effective”, “pharmaceutically effective” or “therapeutically effective” are used herein, unless otherwise indicated, to describe an amount of a compound or composition which, in context, is used to produce or affect an intended result, usually the modulation of autophagy within the context of a particular treatment or alternatively, the effect of a bioactive agent which is coadministered with the autophagy modulator (autotoxin) in the treatment of disease.

The terms “treat”, “treating”, and “treatment”, etc., as used herein, refer to any action providing a benefit to a patient at risk for or afflicted by an autophagy mediated disease state or condition as otherwise described herein. The benefit may be in curing the disease state or condition, inhibition its progression, or ameliorating, lessening or suppressing one or more symptom of an autophagy mediated disease state or condition. Treatment, as used herein, encompasses both prophylactic and therapeutic treatment.

The term “modulator of autophagy”, “regulator of autophagy” or “autostatin” is used to refer to a compound which functions as an agonist (inducer or up-regulator) or antagonist (inhibitor or down-regulator) of autophagy. Depending upon the disease state or condition, autophagy may be upregulated (and require inhibition of autophagy for therapeutic intervention) or down-regulated (and require upregulation of autophagy for therapeutic intervention). In most instances, in the case of cancer treatment or treatment of an autoimmune disease such as rheumatoid arthritis, malaria, antiphospholipid antibody syndrome, lupus, chronic urticaria and Sjogren's disease, with a modulator of autophagy as otherwise described herein, the autophagy modulator is often an antagonist of autophagy. In the case of cancer, the ATG8 modulator may be used alone or in combination with an additional antagonist (inhibitor) of autophagy and/or an additional anticancer agent, which may be used alone or in further combination with an autophagy agonist.

The terms “treat”, “treating”, and “treatment”, etc., as used herein, refer to any action providing a benefit to a patient at risk for or afflicted by an autophagy mediated disease state or condition as otherwise described herein. The benefit may be in curing the disease state or condition, inhibition its progression, or ameliorating, lessening or suppressing one or more symptom of an autophagy mediated disease state or condition. Treatment, as used herein, encompasses both prophylactic and therapeutic treatment.

As used herein, the term “autophagy mediated disease state or condition” refers to a disease state or condition that results from disruption in autophagy or cellular self-digestion. Autophagy is a cellular pathway involved in protein and organelle degradation, and has a large number of connections to human disease. Autophagic dysfunction is associated with cancer, neurodegeneration, microbial infection and ageing, among numerous other disease states and/or conditions. Although autophagy plays a principal role as a protective process for the cell, it also plays a role in cell death. Disease states and/or conditions which are mediated through autophagy (which refers to the fact that the disease state or condition may manifest itself as a function of the increase or decrease in autophagy in the patient or subject to be treated and treatment requires administration of an inhibitor or agonist of autophagy in the patient or subject) include, for example, cancer, including metastasis of cancer, lysosomal storage diseases (discussed hereinbelow), neurodegeneration (including, for example, Alzheimer's disease, Parkinson's disease, Huntington's disease; other ataxias), immune response (T cell maturation, B cell and T cell homeostasis, counters damaging inflammation) and chronic inflammatory diseases (may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), affecting lipid metabolism islet function and/or structure, excessive autophagy may lead to pancreatic β-cell death and related hyperglycemic disorders such as insulin resistance, especially including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), and elevated triglycerides) and metabolic syndrome, liver disease (excessive autophagic removal of cellular entities-endoplasmic reticulum), non-alcohol fatty liver disease (NAFLD), non-alcoholic steatohepatitis (NASH), liver fibrosis, renal disease (apoptosis in plaques, glomerular disease), cardiovascular disease (especially including ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, atherosclerosis and associated conditions such as cardiac and neurological both central and peripheral manifestations including stroke, age-associated dementia and sporadic form of Alzheimer's disease, pre-cancerous states, and psychiatric conditions including depression), stroke and spinal cord injury, arteriosclerosis, infectious diseases (microbial infections, removes microbes, provides a protective inflammatory response to microbial products, limits adapation of authophagy of host by microbe for enhancement of microbial growth, regulation of innate immunity) including bacterial, fungal, cellular and viral (including secondary disease states or conditions associated with infectious diseases), including AIDS and tuberculosis, among others, development (including erythrocyte differentiation), embryogenesis/fertility/infertility (embryo implantation and neonate survival after termination of transplacental supply of nutrients, removal of dead cells during programmed cell death) and ageing (increased autophagy leads to the removal of damaged organelles or aggregated macromolecules to increase health and prolong lire, but increased levels of autophagy in children/young adults may lead to muscle and organ wasting resulting in ageing/progeria).

The term “lysosomal storage disorder” refers to a disease state or condition that results from a defect in lysosomomal storage. These disease states or conditions generally occur when the lysosome malfunctions. Lysosomal storage disorders are caused by lysosomal dysfunction usually as a consequence of deficiency of a single enzyme required for the metabolism of lipids, glycoproteins or mucopolysaccharides. The incidence of lysosomal storage disorder (collectively) occurs at an incidence of about about 1:5,000-1:10,000. The lysosome is commonly referred to as the cell's recycling center because it processes unwanted material into substances that the cell can utilize. Lysosomes break down this unwanted matter via high specialized enzymes. Lysosomal disorders generally are triggered when a particular enzyme exists in too small an amount or is missing altogether. When this happens, substances accumulate in the cell. In other words, when the lysosome doesn't function normally, excess products destined for breakdown and recycling are stored in the cell. Lysosomal storage disorders are genetic diseases, but these may be treated using autophagy modulators (autostatins) as described herein. All of these diseases share a common biochemical characteristic, i.e., that all lysosomal disorders originate from an abnormal accumulation of substances inside the lysosome. Lysosomal storage diseases mostly affect children who often die as a consequence at an early stage of life, many within a few months or years of birth. Many other children die of this disease following years of suffering from various symptoms of their particular disorder.

Examples of lysosomal storage diseases include, for example, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM! Ganliosidosis, including infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs and Wolman disease, among others.

An “inflammation-associated disease” includes an inflammation-associated metabolic disorder, but is not limited to, lung diseases, hyperglycemic disorders including diabetes and disorders resulting from insulin resistance, such as Type I and Type II diabetes, as well as severe insulin resistance, hyperinsulinemia, and dyslipidemia or a lipid-related metabolic disorder (e.g. hyperlipidemia (e.g., as expressed by obese subjects), elevated low-density lipoprotein (LDL), depressed high-density lipoprotein (HDL), and elevated triglycerides) and insulin-resistant diabetes, such as Mendenhall's Syndrome, Werner Syndrome, leprechaunism, and lipoatrophic diabetes, renal disorders, such as acute and chronic renal insufficiency, end-stage chronic renal failure, glomerulonephritis, interstitial nephritis, pyelonephritis, glomerulosclerosis, e.g., Kimmelstiel-Wilson in diabetic patients and kidney failure after kidney transplantation, obesity, GH-deficiency, GH resistance, Turner's syndrome, Laron's syndrome, short stature, increased fat mass-to-lean ratios, immunodeficiencies including decreased CD4⁺ T cell counts and decreased immune tolerance or chemotherapy-induced tissue damage, bone marrow transplantation, diseases or insufficiencies of cardiac structure or function such as heart dysfunctions and congestive heart failure, neuronal, neurological, or neuromuscular disorders, e.g., diseases of the central nervous system including Alzheimer's disease, or Parkinson's disease or multiple sclerosis, and diseases of the peripheral nervous system and musculature including peripheral neuropathy, muscular dystrophy, or myotonic dystrophy, and catabolic states, including those associated with wasting caused by any condition, including, e.g., mental health condition (e.g., anorexia nervosa), trauma or wounding or infection such as with a bacterium or human virus such as HIV, wounds, skin disorders, gut structure and function that need restoration, and so forth.

An “inflammation-associated metabolic disorder” also includes a cancer and an “infectious disease” as defined herein, as well as disorders of bone or cartilage growth in children, including short stature, and in children and adults disorders of cartilage and bone in children and adults, including arthritis and osteoporosis. An “inflammation-associated metabolic disorder” includes a combination of two or more of the above disorders (e.g., osteoporosis that is a sequela of a catabolic state). Specific disorders of particular interest targeted for treatment herein are diabetes and obesity, heart dysfunctions, kidney disorders, neurological disorders, bone disorders, whole body growth disorders, and immunological disorders.

In one embodiment, “inflammation-associated metabolic disorder” includes: central obesity, dyslipidemia including particularly hypertriglyceridemia, low HDL cholesterol, small dense LDL particles and postpranial lipemia; glucose intolerance such as impaired fasting glucose; insulin resistance and hypertension, and diabetes. The term “diabetes” is used to describe diabetes mellitus type I or type II. The present invention relates to a method for improving renal function and symptoms, conditions and disease states which occur secondary to impaired renal function in patients or subjects with diabetes as otherwise described herein. It is noted that in diabetes mellitus type I and II, renal function is impaired from collagen deposits, and not from cysts in the other disease states treated by the present invention.

Mycobacterial infections often manifest as diseases such as tuberculosis. Human infections caused by mycobacteria have been widespread since ancient times, and tuberculosis remains a leading cause of death today. Although the incidence of the disease declined, in parallel with advancing standards of living, since the mid-nineteenth century, mycobacterial diseases still constitute a leading cause of morbidity and mortality in countries with limited medical resources. Additionally, mycobacterial diseases can cause overwhelming, disseminated disease in immunocompromised patients. In spite of the efforts of numerous health organizations worldwide, the eradication of mycobacterial diseases has never been achieved, nor is eradication imminent. Nearly one third of the world's population is infected with Mycobacterium tuberculosis complex, commonly referred to as tuberculosis (TB), with approximately 8 million new cases, and two to three million deaths attributable to TB yearly. Tuberculosis (TB) is the cause of the largest number of human deaths attributable to a single etiologic agent (see Dye et al., J. Am. Med. Association, 282, 677-686, (1999); and 2000 WHO/OMS Press Release).

Mycobacteria other than M. tuberculosis are increasingly found in opportunistic infections that plague the AIDS patient. Organisms from the M. avium-intracellulare complex (MAC), especially serotypes four and eight, account for 68% of the mycobacterial isolates from AIDS patients. Enormous numbers of MAC are found (up to 1010 acid-fast bacilli per gram of tissue), and consequently, the prognosis for the infected AIDS patient is poor.

In many countries the only measure for TB control has been vaccination with M. bovis bacille Calmette-Guerin (BCG). The overall vaccine efficacy of BCG against TB, however, is about 50% with extreme variations ranging from 0% to 80% between different field trials. The widespread emergence of multiple drug-resistant M. tuberculosis strains is also a concern.

M. tuberculosis belongs to the group of intracellular bacteria that replicate within the phagosomal vacuoles of resting macrophages, thus protection against TB depends on T cell-mediated immunity. Several studies in mice and humans, however, have shown that Mycobacteria stimulate antigen-specific, major histocompatibility complex (MHC) class II- or class I-restricted CD4 and CD8 T cells, respectively. The important role of MHC class I-restricted CD8 T cells was convincingly demonstrated by the failure of β2-microglobulin) deficient mice to control experimental M. tuberculosis infection.

As used herein, the term “tuberculosis” comprises disease states usually associated with infections caused by mycobacteria species comprising M. tuberculosis complex. The term “tuberculosis” is also associated with mycobacterial infections caused by mycobacteria other than M. tuberculosis. Other mycobacterial species include M. avium-intracellulare, M. kansarii, M. fortuitum, M. chelonae, M. leprae, M. africanum, and M. microti, M. avium paratuberculosis, M. intracellulare, M. scrofulaceum, M. xenopi, M. marinum, M. ulcerans.

An “infectious disease” includes but is limited to those caused by bacterial, mycological, parasitic, and viral agents. Examples of such infectious agents include the following: staphylococcus, streptococcaceae, neisseriaaceae, cocci, enterobacteriaceae, pseudomonadaceae, vibrionaceae, campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae, systemic mycoses, opportunistic mycoses, protozoa, nematodes, trematodes, cestodes, adenoviruses, herpesviruses, poxviruses, papovaviruses, hepatitis viruses, orthomyxoviruses, paramyxoviruses, coronaviruses, picornaviruses, reoviruses, togaviruses, flaviviruses, bunyaviridae, rhabdoviruses, human immunodeficiency virus and retroviruses.

An “inflammatory disorder” “inflammatory disease state” or “inflammatory condition” includes, but is not limited to, lung diseases, hyperglycemic disorders including diabetes and disorders resulting from insulin resistance, such as Type I and Type II diabetes, as well as severe insulin resistance, hyperinsulinemia, and dyslipidemia (e.g. hyperlipidemia (e.g., as expressed by obese subjects), elevated low-density lipoprotein (LDL), depressed high-density lipoprotein (HDL), and elevated triglycerides) and insulin-resistant diabetes, such as Mendenhall's Syndrome, Werner Syndrome, leprechaunism, and lipoatrophic diabetes, renal disorders, such as acute and chronic renal insufficiency, end-stage chronic renal failure, glomerulonephritis, interstitial nephritis, pyelonephritis, glomerulosclerosis, e.g., Kimmelstiel-Wilson in diabetic patients and kidney failure after kidney transplantation, obesity, GH-deficiency, GH resistance, Turner's syndrome, Laron's syndrome, short stature, increased fat mass-to-lean ratios, immunodeficiencies including decreased CD4⁺ T cell counts and decreased immune tolerance or chemotherapy-induced tissue damage, bone marrow transplantation, diseases or insufficiencies of cardiac structure or function such as heart dysfunctions and congestive heart failure, neuronal, neurological, or neuromuscular disorders, e.g., diseases of the central nervous system including Alzheimer's disease, or Parkinson's disease or multiple sclerosis, and diseases of the peripheral nervous system and musculature including peripheral neuropathy, muscular dystrophy, or myotonic dystrophy, and catabolic states, including those associated with wasting caused by any condition, including, e.g., mental health condition (e.g., anorexia nervosa), trauma or wounding or infection such as with a bacterium or human virus such as HIV, wounds, skin disorders, gut structure and function that need restoration, and so forth.

“Inflammatory disorder” also includes a cancer and an “infectious disease” as defined herein, as well as disorders of bone or cartilage growth in children, including short stature, and in children and adults disorders of cartilage and bone in children and adults, including arthritis and osteoporosis. An “inflammation-associated metabolic disorder” includes a combination of two or more of the above disorders (e.g., osteoporosis that is a sequela of a catabolic state). Specific disorders of particular interest targeted for treatment herein are diabetes and obesity, heart dysfunctions, kidney disorders, neurological disorders, bone disorders, whole body growth disorders, and immunological disorders.

In one embodiment, an “inflammatory disorder” includes central obesity, dyslipidemia including particularly hypertriglyceridemia, low HDL cholesterol, small dense LDL particles and postpranial lipemia; glucose intolerance such as impaired fasting glucose; insulin resistance and hypertension, and diabetes. The term “diabetes” is used to describe diabetes mellitus type I or type II. The present invention relates to a method for improving renal function and symptoms, conditions and disease states which occur secondary to impaired renal function in patients or subjects with diabetes as otherwise described herein. It is noted that in diabetes mellitus type I and II, renal function is impaired from collagen deposits, and not from cysts in the other disease states treated by the present invention.

A “neurodegenerative disorder” or “neuroinflammation” includes, but is not limited to inflammatory disorders such as Alzheimer's Dementia (AD), amyotrophic lateral sclerosis, depression, epilepsy, Huntington's Disease, multiple sclerosis, the neurological complications of AIDS, spinal cord injury, glaucoma and Parkinson's disease.

An “immune disorder” includes, but is not limited to, lupus, multiple sclerosis, rheumatoid arthritis, psoriasis, Type I diabetes, complications from organ transplants, xeno transplantation, diabetes, cancer, asthma, atopic dermatitis, autoimmune thyroid disorders, ulcerative colitis, Crohn's disease, Alzheimer's disease, Sjogren's disease and leukemia.

The following compounds have been identified as autophagy modulators according to the present invention and can be used in the treatment of an autophagy mediated disease state or condition as otherwise described herein. It is noted that an inhibitor of autophagy is utilized where the disease state or condition is mediated through upregulation or an increase in autophagy which causes the disease state or condition and an agonist of autophagy is utilized where the disease state or condition is mediated through downregulation or a decrease in autophagy. The following compounds have been identified as autophagy modulators (autotaxins) in autophagy assays according to the present invention: flubendazole, hexachlorophene, propidium iodide, bepridil, clomiphene citrate (Z,E), GBR 12909, propafenone, metixene, dipivefrin, fluvoxamine, dicyclomine, dimethisoquin, ticlopidine, memantine, bromhexine, ambroxol, norcyclobenzaprine, diperodon, nortriptyline, benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, methimazole, trimeprazine, ethoxyquin, clocortolone, doxycycline, pirlindole mesylate, doxazosin, deptropine, nocodazole, scopolamine, oxybenzone, halcinonide, oxybutynin, miconazole, clomipramine, cyproheptadine, doxepin, dyclonine, salbutamol, flavoxate, amoxapine, fenofibrate, pimethixene and mixtures thereof and their pharmaceutically acceptable salts show activity as agonists or inducers of autophagy in the treatment of an autophagy-mediated disease, whereas tetrachlorisophthalonitrile, phenylmercuric acetate, JQ1, 2-methoxyestradiol, 3-methyladenine (3MA), epigallocatechin gallate (EGCG), 3BDO, 5-aminolevulinic acid, 5-azacytidine, 6-thioguanine, A-317491, A-867744, ABT-737, ABT-751, aceglutamide, acetazolamide, afatinib, capsaicin, actigenin, ascorbic acid, curcumin, resveratrol, SP600125, U0126, Bafiliomycin A1, chloroquine, LY294002, SB202190, SB203580, SC79, autophinib, wortmannin, crocin, harmines, mangiferin, tetrachlorisophthalonitrile, cycloheximide, hydroxychloroquine, Lys05, leupeptin, E64d, pepstatin A, or a pharmaceutically acceptable salt thereof, find use as antagonists or inhibitors of autophagy and can be readily combined with a ATG8 and/or ATG9A modulator as described herein in the treatment of cancer and other disease states and/or conditions rheumatoid arthritis, malaria, antiphospholipid antibody syndrome, lupus, chronic urticaria and Sjogren's disease, among others. All of these compounds will find use as modulators of autophagy in the treatment of cancer, with the antagonists being preferred in such treatment (although inhibitors may be used alone, or in combination with the agonists) and in the case of the treatment of cancer, the inhibitors described above are preferred, alone or in combination with an autophagy agonist as described above and/or an additional anticancer agent as otherwise described herein.

Other compounds which may be used in combination with the autophagy modulators which are described above, include for example, other “additional autophagy modulators” or “additional autostatins” which are known in the art. These can be combined with one or more of the autophagy modulators which are disclosed above to provide novel pharmaceutical compositions and/or methods of treating autophagy mediated disease states and conditions which are otherwise described herein. These additional autophagy modulators include benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, Methimazole, Trimeprazine, Ethoxyquin, Clocortolone, Doxycycline, Pirlindole mesylate, Doxazosin, Deptropine, Nocodazole, Scopolamine, Oxybenzone, Halcinonide, Oxybutynin, Miconazole, Clomipramine, Cyproheptadine, Doxepin, Dyclonine, Salbutamol, Flavoxate, Amoxapine, Fenofibrate, Pimethixene, Earle's balanced salt solution (EBSS), brefeldin A, thapsigargin, tunicamycin, rapamycin, CCI-779, RAD001, AP23576, small molecule enhancer rapamycin 10 (SMER 10), SMER 18, SMER 28, trehalose, lithium chloride, L-690,330, carbamazepine, valproic acid, N-Acetyl-D-sphingosine (C2-ceramide), Penitrem A (tremortin), calpastatin, xestospongin B, pharmaceutically acceptable salts and mixtures thereof.

The term “co-administration” or “combination therapy” is used to describe a therapy in which at least two active compounds in effective amounts are used to treat an autophagy mediated disease state or condition as otherwise described herein, especially cancer either at the same time or within dosing or administration schedules defined further herein or ascertainable by those of ordinary skill in the art. Although the term co-administration preferably includes the administration of two active compounds to the patient at the same time, it is not necessary that the compounds be administered to the patient at the same time, although effective amounts of the individual compounds will be present in the patient at the same time. In addition, in certain embodiments, co-administration will refer to the fact that two compounds are administered at significantly different times, but the effects of the two compounds are present at the same time. Thus, the term co-administration includes an administration in which one active agent (especially a ATG8 and/or ATG9A modulator) is administered at approximately the same time (contemporancously), or from about one to several minutes to about 24 hours or more than the other bioactive agent coadministered with the ATG8 and/or ATG9A modulator (which may be one or more of an additional autophagy inhibitor, and autophagy agonist and an anticancer agent). The additional bioactive agent may be any bioactive agent, but is generally selected from an additional autophagy mediated compound (especially an autophagy inhibitor, although an autophagy agonist may also be used in certain instances), an additional anticancer agent, or another agent, such as a mTOR inhibitor such as pp242, rapamycin, envirolimus, everolimus or cidaforollimus, among others including epigallocatechin gallate (EGCG), caffeine, curcumin or reseveratrol (which mTOR inhibitors may find use as enhancers of autophagy using the compounds disclosed herein and in addition, in the treatment of cancer and other autophagy mediated disease states and/or conditions with an autophagy modulator (inhibitor) as described herein, including in combination with tetrachlorisophthalonitrile, phenylmercuric acetate, JQ1, 2-methoxyestradiol, 3-methyladenine (3MA), epigallocatechin gallate (EGCG), 3BDO, 5-aminolevulinic acid, 5-azacytidine, 6-thioguanine, A-317491, A-867744, ABT-737, ABT-751, aceglutamide, acetazolamide, afatinib, capsaicin, actigenin, ascorbic acid, curcumin, resveratrol, SP600125, U0126, Bafiliomycin A1, chloroquine, LY294002, SB202190, SB203580, SC79, autophinib, wortmannin, crocin, harmines, mangiferin, tetrachlorisophthalonitrile, cycloheximide, hydroxychloroquine, Lys05, leupeptin, E64d, pepstatin A, or a pharmaceutically acceptable salt or mixture thereof, which are inhibitors of autophagy. It is noted that in the case of the treatment of cancer, the use of an autophagy inhibitor is preferred, alone or in combination with an autophagy inducer (agonist) as otherwise described herein and/or a mTOR inhibitor as described above. In certain embodiments, an mTOR inhibitor selected from the group consisting of pp242, rapamycin, envirolimus, everolimus, cidaforollimus, epigallocatechin gallate (EGCG), caffeine, curcumin, reseveratrol and mixtures thereof may be used as the additional bioactive agent (along with the ATG8/ATG9A modulator), alone or in combination with one or more additional bioactive agents, including, for example digoxin, xylazine, hexetidine, sertindole and mixtures thereof, the combination of such agents being effective as autophagy modulators in combination.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

Neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms (cancer) are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, stomach and thyroid; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991). All of these neoplasms may be treated using compounds according to the present invention.

Representative common cancers to be treated with compounds according to the present invention include, for example, prostate cancer, metastatic prostate cancer, stomach, colon, rectal, liver, pancreatic, lung, breast, cervix uteri, corpus uteri, ovary, testis, bladder, renal, brain/CNS (including gliomas, gliobastomas, neuroblastomas), head and neck, throat, thyroid, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. Because of the activity of the present compounds, the present invention has general applicability treating virtually any cancer in any tissue, thus the compounds, compositions and methods of the present invention are generally applicable to the treatment of cancer and in reducing the likelihood of development of cancer and/or the metastasis of an existing cancer.

In certain particular aspects of the present invention, the cancer which is treated is metastatic cancer, a recurrent cancer or a drug resistant cancer, especially including a drug resistant cancer. Separately, metastatic cancer may be found in virtually all tissues of a cancer patient in late stages of the disease, typically metastatic cancer is found in lymph system/nodes (lymphoma), in bones, in lungs, in bladder tissue, in kidney tissue, liver tissue and in virtually any tissue, including brain (brain cancer/tumor). Thus, the present invention is generally applicable and may be used to treat any cancer in any tissue, regardless of etiology. In the present invention, ATG8 and/or ATG9A modulators often are used in conjunction with additional bioactive agents, including additional autophagy modulators (including additional autophagy inhibitors as described herein), additional anticancer agents or alternative cancer therapies, such as radiation therapy, surgery, hormone therapy, immunotherapy, targeted therapy, heat or oxygenation therapy.

The term “tumor” is used to describe a malignant or benign growth or tumefacent.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be an anticancer agent which is distinguishable from a CIAE-inducing anticancer ingredient such as a taxane, vinca alkaloid and/or radiation sensitizing agent otherwise used as chemotherapy/cancer therapy agents herein. In many instances, the co-administration of another anti-cancer compound according to the present invention results in a synergistic anti-cancer effect. Exemplary anti-cancer compounds for co-administration with formulations according to the present invention include anti-metabolites agents which are broadly characterized as antimetabolites, inhibitors of topoisomerase I and II, alkylating agents and microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib).

Exemplary anticancer agents which may be coadministered in combination with one or more chimeric compounds according to the present invention include, for example, antimetabolites, inhibitors of topoisomerase I and II, alkylating agents and microtubule inhibitors (e.g., taxol), among others. Exemplary anticancer compounds for use in the present invention may include everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-M ET inhibitor, a PARP inhibitor, a Cdk inhibitor, an EGFR TK inhibitor, an IGFR-TK inhibitor, an anti-HOF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab (Arzerra), zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vineristine, temozolomide, ZK-304709, seliciclib, PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl], disodium salt, heptahydrate, camptothecin. PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser (But) 6, Azgly 10] (pyro-Glu-His-Trp-Ser-Tyr-D-Ser (Bu t)-Leu-Arg-Pro-Azgly-NH: acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, Alutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others. Other anticancer agents which may be used in combination include immunotherapies such ipilimumab, pembrolizumab, nivolumab, alemtuzumab, atezolizumab, ofatumumab, novolumab, pembrolizumab, and rituximab, among others.

Co-administration of one of the compounds and/or formulations of the invention with another anticancer agent will often result in a synergistic enhancement of the anticancer activity of the other anticancer agent, an unexpected result. One or more of the present formulations comprising an autophagy modulator (autostatin) may also be co-administered with another bioactive agent (e.g., additional anticancer agents, alternative autophagy modulators, especially including autophagy inhibitors), among others as otherwise described herein).

According to various embodiments, the compounds according to the present invention (ATG8 and/or ATG9A modulators and other bioactive agents, when combined with ATG8 and/or ATG9A modulators) may be used for treatment or prevention purposes in the form of a pharmaceutical composition. This pharmaceutical composition may comprise one or more active ingredients as described herein.

As indicated, the pharmaceutical composition may also comprise a pharmaceutically acceptable excipient, additive or inert carrier. The pharmaceutically acceptable excipient, additive or inert carrier may be in a form chosen from a solid, semi-solid, and liquid. The pharmaceutically acceptable excipient or additive may be chosen from a starch, crystalline cellulose, sodium starch glycolate, polyvinylpyrolidone, polyvinylpolypyrolidone, sodium acetate, magnesium stearate, sodium laurylsulfate, sucrose, gelatin, silicic acid, polyethylene glycol, water, alcohol, propylene glycol, vegetable oil, corn oil, peanut oil, olive oil, surfactants, lubricants, disintegrating agents, preservative agents, flavoring agents, pigments, and other conventional additives. The pharmaceutical composition may be formulated by admixing the active with a pharmaceutically acceptable excipient or additive.

The pharmaceutical composition may be in a form chosen from sterile isotonic aqueous solutions for intravenous, intramuscular, intrathecal or intratumor injection, pills, drops, pastes, cream, spray (including aerosols), capsules, tablets, sugar coating tablets, granules, suppositories, liquid, lotion, suspension, emulsion, ointment, gel, and the like. Administration route may be chosen from subcutaneous, intravenous, intrathecal, intratumor (i.e., directly into the tumorous tissue to be treated which is preferred in certain solid tumors), intestinal, parenteral, oral, buccal, nasal, intramuscular, transcutaneous, transdermal, intranasal, intraperitoneal, by inhalation and topical. The pharmaceutical compostions may be immediate release, sustained/controlled release, or a combination of immediate release and sustained/controlled release depending upon the compound(s) to be delivered, the compound(s), if any, to be coadministered, as well as the disease state and/or condition to be treated with the pharmaceutical composition. A pharmaceutical composition may be formulated with differing compartments or layers in order to facilitate effective administration of any variety consistent with good pharmaceutical practice.

The subject or patient may be chosen from, for example, a human, a mammal such as domesticated animal, or other animal. The subject may have one or more of the disease states, conditions or symptoms associated with autophagy as otherwise described herein.

The compounds according to the present invention may be administered in an effective amount to treat or reduce the likelihood of an autophagy-mediated disease and/or condition as well one or more symptoms associated with the disease state or condition, especially cancer. One of ordinary skill in the art would be readily able to determine an effective amount of active ingredient by taking into consideration several variables including, but not limited to, the animal subject, age, sex, weight, site of the disease state or condition in the patient, previous medical history, other medications, etc.

For example, the dose of an active ingredient which is useful in the treatment of an autophagy mediated disease state, condition and/or symptom such as cancer for a human patient is that which is an effective amount and may range from as little as 100 μg or even less to at least about 500 mg to a gram or more, which may be administered in a manner consistent with the delivery of the drug and the disease state or condition to be treated. In the case of oral administration, active is generally administered from one to four times or more daily.

Transdermal patches or other topical administration may administer drugs continuously, one or more times a day or less frequently than daily, depending upon the absorptivity of the active and delivery to the patient's skin. Of course, in certain instances where parenteral administration represents a favorable treatment option, intramuscular administration or slow IV drip may be used to administer active. The amount of active ingredient which is administered to a human patient preferably ranges from about 0.05 mg/kg to about 25 mg/kg, 0.075 mg/kg to about 10 mg/kg, about 0.1 mg/kg to about 7.5 mg/kg, about 0.25 mg/kg to about 6 mg/kg., about 1.25 to about 5.7 mg/kg.

The dose of a compound according to the present invention may be administered at the first signs of the onset of an autophagy mediated disease state, condition or symptom such as cancer. For example, the dose may be administered for the purpose of inhibiting cancer growth, increasing the likelihood of remission of cancer, reducing the likelihood of metastatis and/or recurrence of cancer. The dose of active ingredient may be administered at the first sign of relevant symptoms prior to diagnosis, but in anticipation of the disease or disorder or in anticipation of decreased bodily function or any one or more of the other symptoms or secondary disease states or conditions associated with an autophagy mediated disorder to condition.

These and other aspects of the invention are described further in the following illustrative examples which are presented herein below.

### EXAMPLES

### Materials and Methods

Antibodies and reagents. The following antibodies and dilutions were used: mouse anti-Flag (mAb Sigma; F1804, used at 0.5 μg/ml for IP, 1:1,000 for WB); rabbit anti-GFP (Abcam; ab290; 0.5 μg/ml for IP and 1:4,000 for WB); mouse anti-DNA antibody (Progen; 61014; 1:300 for IF); Mouse anti-LC3 (MBL; 152-3, 1:400 for IF) Anti-ATG9A (CST; #13509, 1:500 for WB), Rabbit anti-IQGAP1 (CST; 20648s, 1:1000 for WB), Rabbit anti-MTCO2 (Abcam; ab110258, WB-1:1000), Rabbit anti-LC3B (CST; 27755s, WB-1:1000), NDP52 (CST; 60732s, 1:1000 for WB), p62 (BD Bioscience; 610833, 1:1000 for WB), Rabbit anti-Parkin (Abcam; ab15954, 1:500 for WB). Rabbit anti-CHMP2A (Proteintech, 10477-1-AP, 1:500), Dynabeads Protein G (Thermo Fisher Scientific; 10003D, 50 μl/ml for IP). We also used the following reagents: Bafilomycin A1 (BafA1, InvivoGen; 13D02-MM); CCCP (Sigma, C2759), PP242 (Sigma, P0037), Lipofectamine 2000 (Thermo Scientific, 11668019); Lipofectamine RNAiMAX (Thermo Scientific, 13778150); Goat anti-mouse IRDye 680 (LI-COR, 925-68020); Goat anti-rabbit IRDye 800 (LI-COR, 926-32211); membrane impermeant ligand (MIL), HaloTag ligand Alexa Fluor 660 (Promega, G8471); membrane permeant ligand (MPL), HaloTag ligand TMR (Promega, G8251). Nano-Glo® HiBiT Extracellular Detection System (Promega, N2420), Saponin (Sigma, S4521). DMEM (Gibco, #11995040), and Penicillin-Streptomycin (1,000U/ml; Gibco, #15140122). OptiMEM and EBSS medias from Life Technologies.

Cell culture. HEK 293T were obtained from ATCC, Huh7 cells were from Rocky Mountain Laboratory. HeLa YFP-Parkin was from Dr. Richard Youle¹. HeLa^{WT}, HeLa^{HexaKO}, HeLa^{LC3TKO} HeLa^{GABATKO} were grown as mentioned previously². Huh7^{ATG9AKO}, Huh7-HT-LC3B^{ATG9AKO}, Huh7-HT-LC3B^{VPS37BKO}, HeLa^{HexaKO} HT-LC3B, HeLa^{LC3TKO} HT-LC3B, HeLa^{GABATKO} HT-LC3B, Huh7-HT-LC3B^{VPS37AKO} and respective parental cells were grown in DMEM supplemented with 10% fetal bovine serum and antibiotics. For starvation-induced autophagy, cells were washed three times in PBS and incubated for 90 min in EBSS. For mitophagy, cells were incubated with 20 μM CCCP in full medium for 6 h.

Plasmids and siRNA transfection. pDest-FLAG-ATG9A³, pDest-FLAG-VPS37A^{Full}, pDest-FLAG-UEV^{VPS37A}, pDest-FLAG-VPS37A^{deltaCEV}, pDest-FLAG-deltaN (1-90), pDest-FLAG-deltaN (1-20), pDest-FLAG-VPS37A N ter-EGFP, pDest-GFP-LC3A, pDest-GFP-LC3C, pDest-GFP-GABARAP, GST-LC3A, GST-LC3B, GTS-LC3C, GST-GABARAP-GST-GABRAP-L1, GST-GABARAP-L2, were first cloned into pDONR221 (Gateway Technology cloning vector, Thermo Scientific) using a BP cloning reaction and the expression vectors were then made by a LR cloning reaction (Gateway, Thermo Fisher) in (pDEST) destination vectors. Plasmid constructs were verified by DNA-sequencing (Genewiz). Plasmids were transfected using Lipofectamine 2000 (Thermo Fisher Scientific, #11668019). For siRNAs, VPS37A (Invitrogen; 127976, 10 nM), CHMP2A (Dharmacon; M-020247-00-0005, 10 nM), and siIQGAP1 (Dharmacon; M-004694-02-005, 10 nM) were delivered into the cells using Lipofectamine RNAiMAX (Thermo, #13778150).

Generation of HaloTag-LC3B (HT-LC3B) stable cell lines. HeLa^{WT}, HeLa^{HexaKO}, HeLa^{LC3TKO}, and HeLa^{GABATKO}, Huh7^{WT} cells were infected with Halo/hMAPILC3B-lentivirus particle and after 48 h of infection cells were incubated with puromycin (2 μg/ml) for 1 week to select the HaloTag-HT-LC3B stable clones. Huh7-HT-LC3B cell were used to generate Huh7-HT-LC3B^{ATG9AKO} Huh7-HT-LC3B^{VPS37BKO}, Huh7-HT-LC3B^{VPS37AKO} with 400 μg of hygromycin and HeLa-YFP-Parkin cells were used to generate HeLa-YFAP-Parkin^{ATG9AKO}, HeLa^{WT} cells were used to generate HeLa^{VPS37BKO} cell line (2 μg/ml) for 1 week to select the complete KO clones.

Generation of CRISPR mutant cells. ATG9A CRISPR knockouts in Huh7 were generated as described⁴⁻⁶. HeLa-YFP Parkin ATG9A CRISPR KO (HeLa-YFP Parkin^{ATG9AKO}) and Huh7 ATG9A CRISPR KO (Huh7^{ATG9AKO}) cells were generated by transduction of two ATG9A CRISPR-Cas9 gRNAs (GACCCCCAGGAGTGTGACGG). For VPS37B and VPS37A knock out cells, the lentiviral vector carrying both Cas9 enzyme and a gRNA targeting VPS37B and VPS37A CRISPR cells were generated for this study (GGAAACTGGCCCACATGCGA) were transfected into HEK293T cells together with the packaging plasmids psPAX2 and pCMV-VSV-G at the ratio of 5:3:2. Two days after transfection, the supernatant containing lentiviruses was collected and used to infect HeLa or Huh7 cells. 48 h hours after infection, the cells were treated with puromycin (2 μg/ml) for one week to select knockout cells, HeLa^{WT} and HeLa^{VPS37BKO}, Huh7^{WT} and Huh7^{VPS37BKO}. The knockouts were confirmed by western blotting. For Huh7-HT-LC3B stable cell line, knockouts for VPS37B, VPS37A and ATG9A were generated with hygromycin (final concentration of 400 μg/ml).

Immunoblotting and co-immunoprecipitation assays. Immunoblotting and co-immunoprecipitation (Co-IP) were performed as described previously⁴. For Co-IP, 10 cm dish cells were transfected with 12 μg of plasmids, wherever stated, and lysed in NP-40 buffer containing protease inhibitor cocktail (Roche, cat #11697498001) and PMSF (Sigma, cat #93482). Lysates were mixed with 5 μg antibody and incubated at 4° C. for overnight followed by incubation with Dynabeads protein G (Life Technologies) for 4 h, at 4° C. Beads were washed three times with PBS and then boiled with SDS-PAGE buffer for analysis of interacting protein by immunoblotting.

GST pull-down Assay. Recombinant GST and GST-fusion proteins were produced in Escherichia coli SoluBL21 (Genlantis, #C700200) by inducing expression in overnight cultures with 50-75 μg/mL isopropyl β-D-1-thiogalactopyranoside (IPTG). Expressed proteins were purified by immobilization on Glutathione Sepharose 4 Fast Flow beads (GE Healthcare, #17-5132-01). For GST pull-down assays, myc-tagged proteins were in vitro translated and radiolabeled with [35S]-methionine using the TNT T7 Reticulocyte Lysate System (Promega, #14610). Ten μL of In vitro translated proteins were precleared to reduce nonspecific binding with 10 μL of empty Glutathione Sepharose beads in 100 μL of NETN buffer (50 mM Tris pH 8.0, 150 mM NaCl, 1 mM EDTA, 0.5% NP-40) supplemented with cOmplete™ EDTA-free Protease Inhibitor Cocktail (Roche, #1183617001) for 30 min at 4° C. This was followed by incubation with purified GST or GST-fusion proteins for 1-2 h at 4° C. The mixture was washed 5 times with NETN buffer by centrifugation at 2,500 g for 2 min followed by addition of 2×SDS gel-loading buffer (100 mM Tris pH 7.4, 4% SDS, 20% Glycerol, 0.2% Bromophenol blue and 200 mM dithiothreitol (DTT) (Sigma, #D0632) and heating for 10 min. The proteins were then resolved by SDS-PAGE and the gel stained with Coomassie Brilliant Blue R-250 Dye (Thermo Fisher Scientific, #20278) to visualize the GST and GST-fusion proteins. The gel was vacuum-dried and radioactive signal detected by Bioimaging analyzer BAS-5000 (Fujifilm).

HCM: MIL/MPL assay for assessment of autophagic membrane sealing. HaloTag-LC3B stable cell lines were seeded in 96 well plate at the density of 8,000 cells/well. After 18-24 h cells were induced for autophagy (EBSS for 90 min) or mitophagy (20 μM CCCP for 6 h). After induction, cells were incubated with Membrane Impermeant Ligand (MIL: Alexa Fluor-660 663_{Ex}/690_{Em}, 1 μM, Promega, G8471) prepared in 1×MAS buffer (220 mM mannitol, 70 mM sucrose, 10 mM KH2PO4, 5 mM MgCl2, 2 mM HEPES, 1 mM EGTA, from Sigma) containing XF-PMP (Agilent; 102504-100, 1.5 nM for Huh7 and 4 nM for HeLa cells) at 37° C. for 15 min. Cells were washed twice with 1×PBS and fixed with 4% paraformaldehyde (PFA, Sigma) in PBS for 5 min at RT. Cells were again washed twice with 1×PBS and incubated with Membrane-Permeant Ligand (MPL: TMR 5 μM, 555_{Ex}/585_{Em}, Promega, G8251) for 30 min at RT. Cells were washed once with 1×PBS and stained with Hoechst 33342 (Thermo Scientific, H3570, 1:1000) for 10 min at RT. Cells were washed twice and plate was scanned by High Content Microscopy (HCM) for automated image acquisition and quantification, iDEV software (Thermo) in 96-well plates⁵.

LC3 puncta count by HCM. High content microscopy (HCM) was performed as described previously⁵. Briefly, cells were plated in 96 well at a density of 8000 cells/well, cells were stimulated for autophagy or mitophagy followed by fixation with 4% PFA in PBS for 5 min. Cells were permeabilized and blocked with 0.1% saponin in 3% BSA for 30 min, followed by incubation with primary antibody (anti-LC3 produced in mouse, MBL 152-3) overnight at 4° C. and secondary antibody for Alexa Fluor 488 for 1 h at RT.

High content microscopy for mitophagy detection. HeLa-YFP Parkin^{WT} or HeLa-YFP Parkin^{ATG9AKO} cells were plated in 96 well plates at the density of 8000 cells/well. After 18-24 h cells were treated with 20 μM CCCP for 6 h or 5 μM Oligomycin/10 μM Antimycin for 18 h. Cells were fixed with 4% PFA and permeabilized and blocked in 3% BSA with 0.1% saponin for 30 min followed by incubation with primary antibody anti-mouse mtDNA overnight at 4° C. and secondary antibody Alexa Fluor 568 for 1 h. High content microscopy with automated image acquisition and quantification was carried out using a Cellomics HCS scanner and iDEV software (Thermo) in 96-well plates⁵.

SolVi—In vitro assessment of autophagic membrane sealing. In vitro sealing of lipid bilayer (SolVit) was developed for HCM platform with modification from the previously described assay for in vitro fusion⁷⁻⁹. For Solvit, HeLa^{HexaKO} HT-LC3B, HeLa^{WT} and HeLa^{HexaKO} cells were seeded in 100 mM dish at the density of 8-10×10⁵ cells/plate and incubated for 18-24 h. Next day cells were induced for autophagy for 90 min with EBSS. Cells were harvested and homogenized with B1 buffer (20 mM Hepes-KOH, pH 7.2, 400 mM sucrose, and 1 mM EDTA). Homogenates were centrifuged at 12,000 g for 30 min at 4° C. and post nuclear supernatant (PNS) containing HeLa^{WT} or HeLa^{HexaKO} (Donors) and HeLa^{HexaKO} HT-LC3B (Acceptor) membranes were mixed for 60 min in the presence or absence of ATP and ATP regenerative system at 37° C. Control samples were left on ice. After ATP regeneration, the samples were stained with MIL for 15 min at 37° C. Samples were fixed with 2% paraformaldehyde in PBS for 15 min and sequentially stained with MPL for 30 min. samples were centrifuged at 43,000 g for 1 h at 4° C. Supernatants were discarded and pellets were resuspended in 100 μl of mounting media+100 μl B1 buffer and dispensed in 96 well plates (40 μM/well, at least 5 wells per sample). The plates were centrifuged at 1000 g for 10 min to allow settling down of the membranes to bottom of the plate. The plates were scanned in Cell Insight CX7 High-Content Screening (HCS) Platform (Thermo). A minimum of 10,000 objects were scanned per well and 5 wells were used for analysis.

In vitro complementation assays in the SolVit system. For observing in vitro complementation effect, the PNS was extracted from either HeLa^{LC3TKO} or HeLa^{GABATKO} stably expressing HT-LC3B (acceptor) and combined with donor PNS preparations from HeLa_{WT}, HeLa^{LC3TKO} or HeLa^{GABATKO} and incubated at 37° C. in in the presence or absence of ATP for 1 h. After incubation samples were incubated with MIL for 15 min at 37° C. Samples were fixed with 2% of PFA for 10 min at RT and sequentially stained with MPL for 30 min at RT in dark. Samples were centrifuged at 43,000 g for 1 h at 4° C. Supernatants were discarded and pellets were resuspended in 100 μl of mounting media+100 μl B1 buffer and dispensed in 96 well plates (40 μM/well, at least 5 wells per sample). The plates were centrifuged at 1000 g for 10 min to allow settling down of the membranes to bottom of the plate. The plates were scanned in Cell Insight CX7 High-Content Screening (HCS) Platform (Thermo). A minimum of 10,000 objects were scanned per well and 5 wells were used for analysis.

Luciferase assay. HiBit is an 11 amino acid high affinity binding peptide which rapidly binds to Large Bit (LgBit) luciferase subunit (Promega, N2420). For luciferase assay, cells were seeded in 60 mM dish and incubated for 12-16 h. After incubation, cells were transfected with 8 μg LC3HiBit reporter plasmid in 8 μl of Lipofectamine 2000 and incubated overnight. Next day cells were treated with DMSO or 2 μM PP242 for 6 h, after incubation cells were incubated with Nano Glo non-Lytic substrate (1:50) along with LgBit protein (1:100) and 4 nm plasma membrane permeabilizer (PMP), substrate was mixed with equal volume of media in each well, reagents were added and equilibrated for 10 min at room temperature. Bio luminescence was measured by reading plate with luminometer (BioTek, Synergy hTx MultiMode, Microplate Reader, 19042612).

Protease protection assay. Huh7^{WT} or Huh7^{ATG9AKO}, Huh7^{VPS37BKO} and HeLa^{WT} and HeLa^{HexaKO} cells, seeded into 100 mM dishes and induced for autophagy by incubation in EBSS for 90 min in presence of 100 nM bafilomycin A1. After treatment, cells were homogenized in homogenization buffer containing 1 M HEPES-KOH (pH 7.5), 4 g of D-mannitol, 2.4 g of sucrose, and dilute with 100 ml distilled water. Cells were harvested and centrifuged at 500 g at 4° C., the post-nuclear supernatant was collected and was equally divided into three parts, one of the samples was left untreated, and the other two were incubated with 50 μg/ml Proteinase K in presence or absence of Triton X-100 (TX-100; 1%) for 30 min on ice. All samples were then subjected to TCA precipitation for 30 min, and protein pellets were resuspended in the 50 μl of 2× sample buffer. Approximately 40-60 μg of each sample was analyzed by immunoblotting.

AlphaFold prediction. Using ColabFold: AlphaFold2 using MMseqs2, a recently developed machine-learning-based protein and protein complex structure prediction program with the sequence from UniProt entries, we modeled interactions between VPS37A (VPS37A^{deltaCEV}) with LC3A, and GABARAP, to determine the binding interactions between VPS37A^{deltaLEV} and each of the proteins respectively. The following protein sequences from UniProt¹⁰ were used: VPS37A (ID: Q8NEZ2), LC3A (ID: QH9H492) and GABARAP (ID: 095166). The VPS37A^{deltaCEV} sequence was generated by removing residues 23-217 (UEV domain). Within ColabFold, the MMseq2 (UniRef+Environmental) was chosen for MSA (multiple sequence alignment) and unpaired+paired was chosen as the pair mode. The complex structure predictions were performed using the multimer-vl option. To probe the convergence of the ColabFold predictions, the predictions were run using 3, 24, and 48 recycles, where recycling means the prior model predictions are placed back into the model to further refine the structure¹¹. Moreover, the comparison between the 24 and 48 recycles shows much lower values (all except one were below 1.5 Å). This demonstrates that the predictions are likely to have reached convergence upon running for 48 recycles. This information not only reveals that the proteins have their correct structure predicted but also provides insight to future ColabFold predictions that 48 recycles is optimal for these protein complexes to reach convergence of their predicted structures. The root-mean-square deviation (RMSD) and binding interface analysis were calculated using the software package Bio3D^{12, 13}. ColabFold produces five models which are independently executed from the same set of inputs. The predictions are then re-ranked according to the predicted Local Distance Difference Test score (pLDDT) for each model. ColabFold produces an IDDT graph for each run, which shows all five ranked models and their predicted IDDT C_{α} score at each residue. The ranking corresponds to where rank 1 is the best model and rank 5 is the poorest model out of those produced.

### Ultrastructural Analysis.

Transmission electron microscopy. For transmission electron microscopy analysis, HeLa^{WT} and Hexa^{KO} cells were grown in 6 well plates until they became semi-confluent. After 90 min of EBSS starvation, cells were fixed with 2% glutaraldehyde (EM grade) in 0.2 M HEPES, pH 7.4. After 30 min, the cells were scraped under a small volume of fixative and transferred to 1.5 mL tubes to be spun at full speed for 10 min at room temperature to get a firm pellet. The pellets continued the fixation for up to 2 h. Cells were post-fixed in 1% OsO4, dehydrated in ethanol and embedded in Epon resine. Thin sections were cut using an ultramicrotome, collected onto electron microscopy cupper grids, and stained with uranyl acetate and lead citrate. In order to count autophagic organelles (autophagosomes/phagophores, autolysosomes, amphisomes, MVBs, late endosomes/lysosomes), 93 images of each sample were taken at primary magnification of 4000×, using uniform random sampling. The images were zoomed on computer screen. Autophagic compartments were counted, and the cytoplasmic area was estimated by point counting and exhibited as profiles per square μm¹⁴.

Immuno Electron Microscopy. For labeling with rabbit GFP antibody (Abcam; ab290, 1:500), two different protocols were used, pre-embedding and post-embedding immunoEM. For pre-embedding immunoEM, HeLa^{HexaKO} cells were grown in glass cover slips in 6 cm dishes until they became sub-confluent, and transfected with 8 μg of plasmid encoding GFP-LC3A overnight. The cells were starved for 90 min with EBSS. For pre-embedding, cells were fixed in PLP-fixative (2% formaldehyde—0.01 M sodium periodate—0.075M lysine—0.037M Na—phosphate buffer, pH 7.4) for 2 h at RT. After washing in 0.1M Na-phosphate buffer, the cells were permeabilized in buffer A (0.01% saponin3/0.1% BSA/0.1 M Na—PO4), incubated in rabbit anti-GFP diluted in buffer A for 1 h at RT, and washed in buffer A. After incubation in the secondary antibody (goat anti-rabbit IgG coupled to 1.2 nm gold, Nanoprobes #2004 Nanogold®-Fab′ Goat anti-Rabbit IgG) the cells were washed in buffer A and 0.1M Na-phosphate buffer, fixed in 1% glutaraldehyde for 10 min, and quenched with 50 mM glycine in phosphate buffer. The 1.2-nm gold particles were then silver enhanced using HQ SILVER Enhancement kit (Nanoprobes, #2012) according to manufacturer's instructions, and the cells were then embedded in Epon and thin sectioned as described above.

For post-embedding, cells were fixed with 4% paraformaldehyde, 0.1% glutaraldehyde (EM grade) diluted in 0.1M Hepes buffer, pH 7.4, for 2 h at RT, scraped off the culture dish under the fixative, and pelleted. The pellets were washed in PBS and embedded in 10% gelatine in PBS. Small cubes were cut from the cell pellet, infiltrated in 15% PVP-1.7M sucrose in PBS, mounted on sample holders, and frozen in liquid nitrogen. Thin sections were cut at −110 C and picked up to Pioloform-carbon-coated nickel grids using 2.3 M sucrose in PBS. The sections were immunolabeled with rabbit anti-GFP and goat anti-rabbit IgG-10 nm gold (British BioCell EM.GAR10), and embedded in a mixture of 1.5% methyl cellulose and 0.4% uranyl acetate. The sections were photographed using Jeol 1400Plus transmission electron microscope.

Super-resolution microscopy. Hela^{HexaKO} cells were plated on 25 mM coverslips in 6 well plate (Warner instruments) and allowed to attach for 12-18 h, by incubating at 37° C., after incubation cells were starved in EBSS for 90 min. Cells were stained with MIL in presence of PMP for 15 min. After 15 min. The cells were washed with 1×PBS and then chemically fixed in two steps. The cells were first treated with 0.6% paraformaldehyde (PFA), 0.1% glutaraldehyde (GA), in PBS for 60 seconds. The cells were then fixed for 2-2.5 h in 3% PFA and 1% GA in PBS. The coverslip was mounted on Attofluor cell chamber with 1.5 mL of imaging buffer which consist of an enzymatic oxygen scavenging system and primary thiol: 50 mM Tris, 10 mM NaCl, 10% w/v glucose, 168.8 U/ml glucose oxidase (Sigma #G2133), 1404 U/ml catalase (Sigma #C9332), and 1M 2-aminoethanethiol (MEA), pH 8.50. The sealed Attofluor chamber was placed at room temperature to allow the oxygen-scavenging reaction to progress for 30 minutes before the imaging. A custom-built sequential microscope controlled by custom written software (github.com/LidkeLab/matlab-instrument-control) in MATLAB (MathWorks Inc.) was used to perform dSTORM imaging. A high power 647 nm laser (2RU-VFL-P-500-647-BIR, MPB Communications) was used as an excitation laser and 405 nm diode laser (DL5146-101S, Thorlabs) was used to accelerate the dark to fluorescent state transition. A 100× silicon oil immersion objective (UPLSAPO100XS, Olympus) was used to collect emitted fluorescent light. 708/75 nm bandpass filter (FF01-708/75-25-D, Semrock) was placed in the emitted fluorescence light path. An sCMOS camera (C11440-22CU, Hamamatsu) was used to detect emitted fluorescence light. At first brightfield reference image was taken using 660 nm LED (M660L3, Thorlabs) illumination lamp and saved. During data acquisition, 15 sequences of 6,000 frames (a total of 90,000) were collected at 100 Hz. The saved brightfield reference image was used to realign each cell during second round of imaging. Data was analyzed via a 2D localization algorithm based on maximum likelihood estimation (MLE)^{15, 16}. The low-quality and false localizations were filtered out by placing several thresholds on minimum number of detected photons, PSF width, localization error, and goodness of the fit of PSF model as defined by a p-value¹⁶. A frame connection algorithm [3] was applied to combine repeated localizations of the same emitter for the same blinking event. This is followed by drift correction algorithm¹⁷ to correct for residual sample drift. The accepted emitters were used to reconstruct the Gaussian super-resolution images. Each accepted emitter was represented by a 2D Gaussian function with localization precisions (σₓ, σ_{y}) calculated from Cramér-Rao Lower Bounds (CRLB).

APEX2-labeling and streptavidin enrichment for LC-MS/MS analysis. HEK293T^{APEX2-ATG9A} cells were incubated in 500 μM biotin-phenol (AdipoGen) in complete media before inducing plasma membrane damage. For digitonin treatment, 100 μg/ml digitonin diluted in complete media was added on the cells for 1 min. Cells were washed once in complete media before adding back biotin-phenol media. For SLO treatment, cells were washed at 37° C. with Ca²⁺-free HBSS containing 5 mM EGTA followed by two more washes in Ca²⁺-free HBSS. SLO was reduced by 10 mM DTT 5 min at room temperature before dilution in Ca²⁺-free HBSS (200 U/ml) and added on target cells for 10 min at 37° C. Cells were then washed once in complete media before adding biotin-phenol media. For GBI treatment, ˜1.6 g of beads were gently poured on the 10 cm petri dish containing the cells. The beads were agitated over the cells for 1 min on a rotator platform at 160 rpm. A Imin pulse with 1 mM H₂O₂ at room temperature was stopped with quenching buffer (10 mM sodium ascorbate, 10 mM sodium azide and 5 mM Trolox in PBS). All samples were washed twice with quenching buffer, and twice with PBS.

For LC-MS/MS analysis, cell pellets were lysed in 500 μl ice-cold lysis buffer (6 M urea, 0.3 M NaCl, 1 mM EDTA, 1 mM EGTA, 10 mM sodium ascorbate, 10 mM sodium azide, 5 mM Trolox, 1% glycerol and 25 mM Tris-HCl, pH 7.5) for 30 min by gentle pipetting. Lysates were clarified by centrifugation and protein concentrations determined using Pierce 660 nm protein assay reagent. Streptavidin-coated magnetic beads (Pierce) were washed with lysis buffer. 1 mg of each sample was mixed with 100 μl of streptavidin beads. The suspensions were gently rotated at 4° C. for overnight to bind biotinylated proteins. The flow-through after enrichment was removed and the beads were washed in sequence with 1 ml IP buffer (150 mM NaCl, 10 mM Tris-HCl, pH 8.0, 1 mM EDTA, 1 mM EGTA, 1% Triton X-100) twice; 1 ml IM KCl; 1 ml of 50 mM Na₂CO₃; 1 ml 2 M urea in 20 mM Tris HCl, pH 8.0; and 1 ml IP buffer. Biotinylated proteins were eluted, 10% of the sample processed for immunoblotting and 90% of the sample processed for mass spectrometry.

LC-MS/MS. Peptides were trapped on a Thermo PepMap trap and separated on an Easy-spray 100 μm×25 cm C18 column using a Dionex Ultimate 3000 nUPLC at 200 nl/min. Solvent A=0.1% formic acid, Solvent B=100% Acetonitrile 0.1% formic acid. Gradient conditions=2% B to 50% B over 60 minutes, followed by a 50%-99% B in 6 minutes and then held for 3 minutes than 99% B to 2% B in 2 minutes and total run time of 90 minutes using Thermo Scientific Fusion Lumos mass spectrometer running in Data Independent Acquisition (DIA) mode. Six-gas phase fractionated (GFP) chromatogram library injections were made using staggered 4 Da isolation widows. GFP1=400-500 m/z, GFP2=500-600 m/z, GFP3=600-700 m/z, GFP4=700-800 m/z, GFP5=800-900 m/z, GFP6=900-1000 m/z, mass spectra were acquired using a collision energy of 35, resolution of 30 K, maximum inject time of 54 ms and a AGC target of 50K.

Each individual sample was run in DIA mode using the same settings as the chromatogram library runs except using staggered isolation windows of 12 Da in the m/z range 400-1000 m/z. DIA data was analyzed using Scaffold DIA v.2.0.0 (Proteome Software, Portland, OR, USA). Raw data files were converted to mzML format using ProteoWizard v.3.0.11748¹⁸.

Chromatogram Library Creation. The Reference Spectral Library was created by EncyclopeDIA v.0.9.2. Chromatogram library samples were individually searched against the Pan human library http://www.swathatlas.org/with a peptide mass tolerance of 10.0 ppm and a fragment mass tolerance of 10.0 ppm. Variable modifications considered were: Oxidation of Methionine and Carbamidomethyl of cysteine. The digestion enzyme was assumed to be Trypsin with a maximum of 1 missed cleavage site(s) allowed. Only peptides with charges in the range [2 . . . 3] and length in the range [6 . . . 30] were considered. Peptides identified in each search were filtered by Percolator (3.01.nightly-13-655e4c7-dirty))¹⁹⁻²¹ to achieve a maximum FDR of 0.01. Individual search results were combined, and peptides were again filtered to an FDR threshold of 0.01 for inclusion in the reference library.

Spectral library search. Analytic samples were aligned based on retention times and individually searched against the chromatogram library created from the six-gas phase fractionated runs described above with a peptide mass tolerance of 10.0 ppm and a fragment mass tolerance of 10.0 ppm. Variable modifications considered were: Oxidation of Methionine and Carbamidomethyl of cysteine. The digestion enzyme was assumed to be Trypsin with a maximum of 1 missed cleavage site(s) allowed. Only peptides with charges in the range [2 . . . 3] and length in the range [6 . . . 30] were considered. Peptides identified in each sample were filtered by Percolator (3.01.nightly-13-655e4c7-dirty) to achieve a maximum FDR of 0.01. Individual search results were combined and peptide identifications were assigned posterior error probabilities and filtered to an FDR threshold of 0.01 by Percolator (3.01.nightly-13-655c4c7-dirty).

Quantification and criteria for protein identification. Peptide quantification was performed by EncyclopeDIA v. 0.9.2. For each peptide, the five highest quality fragment ions were selected for quantitation. Proteins that contained similar peptides and could not be differentiated based on MS/MS analysis were grouped to satisfy the principles of parsimony. Proteins with a minimum of 2 identified peptides were thresholder to achieve a protein FDR threshold of 1.0%.

BioWeB assay: APEX2-labeling and streptavidin enrichment for immunoblotting analyses. For LC-MS/MS analysis, HEK293T^{APEX2-ATG9A} cells were treated as described above. Cells were lysed in 500 μl of ice-cold NP-40 buffer for 30 min on ice. Lysates were clarified by centrifugation and protein concentrations determined using Pierce 660 nm protein assay reagent. One mg of each sample was mixed with 100 μl of streptavidin magnetic beads (Pierce). The suspensions were gently rotated at 4° C. overnight to bind biotinylated proteins. The flow-through after enrichment was removed and the beads were washed in sequence with 1 ml IP buffer twice; 1 ml 1M KCl; 1 ml of 50 mM Na2CO3; 1 ml 2M urea in 20 mM Tris-HCl, pH8; and 1 ml IP buffer. Biotinylated proteins were eluted with 2×Laemmli sample buffer (Bio-Rad) and subjected to immunoblot analysis.

Statistical analyses. Statistical significance was determined by ANOVA followed by Tukey's multiple comparison test. All values are mean±SD, n=3 biologically independent experiments, each HCM experiment: >500 valid primary objects/cells per well, ≥6 wells/sample, up to 60 fields/well.

Data and materials availability. All primary data and unique materials (at a reasonable cost) are available on request from RJ and VD. Commercial or previously published materials are appropriately identified. MS Raw data and DIA Scaffold results are available from the MassIVE proteomics repository (MSV000084519) and Proteome Exchange PXD016084.

### Results

GABARAP and LC3A but not LC3B Interact with ESCRT-I Component PS37A

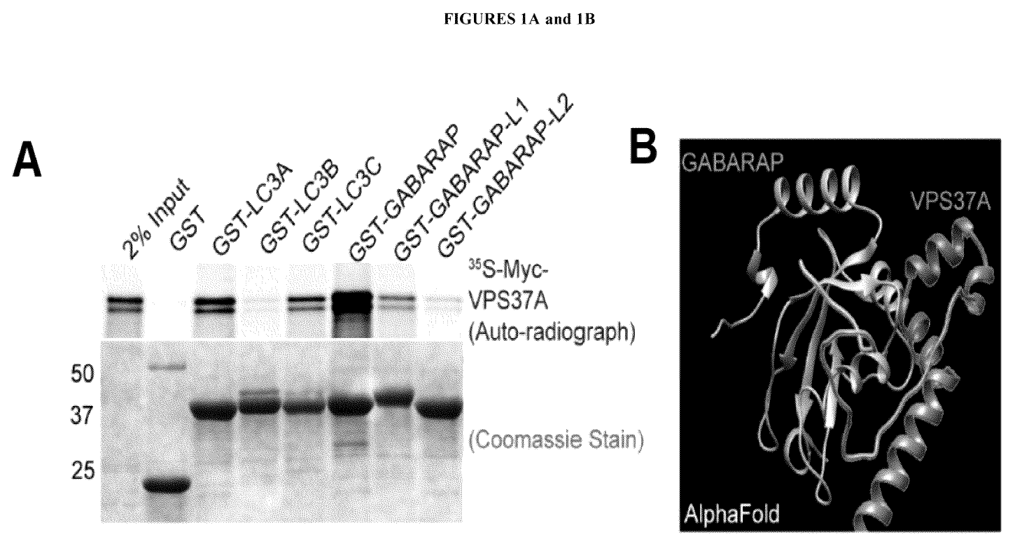

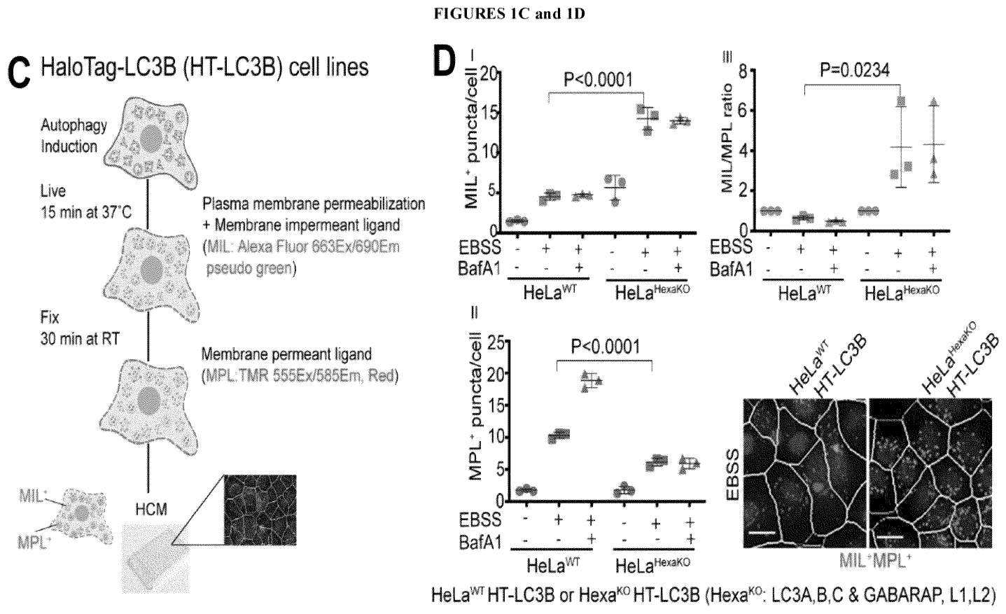

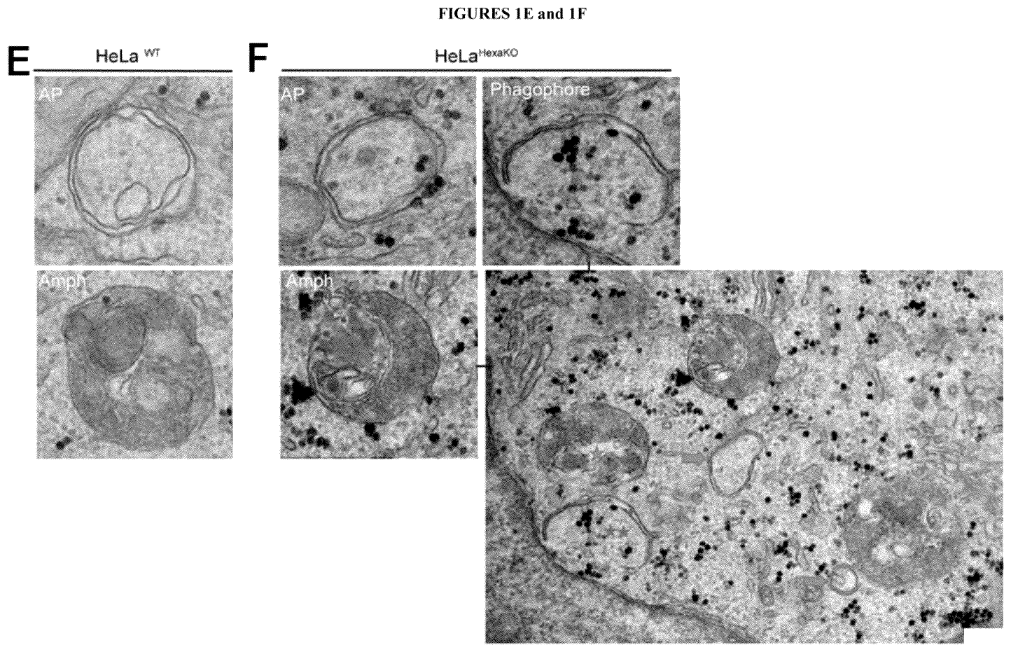

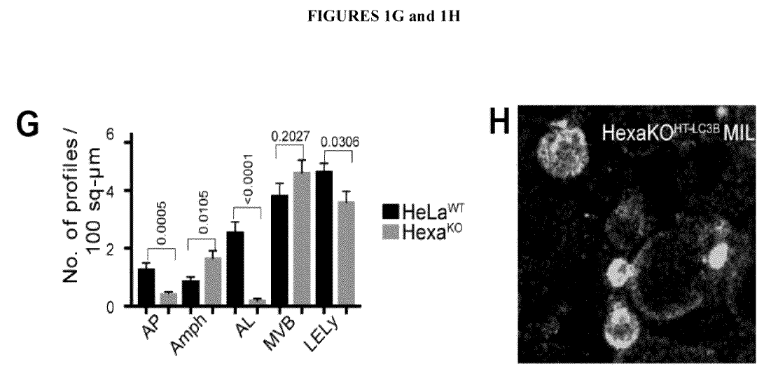

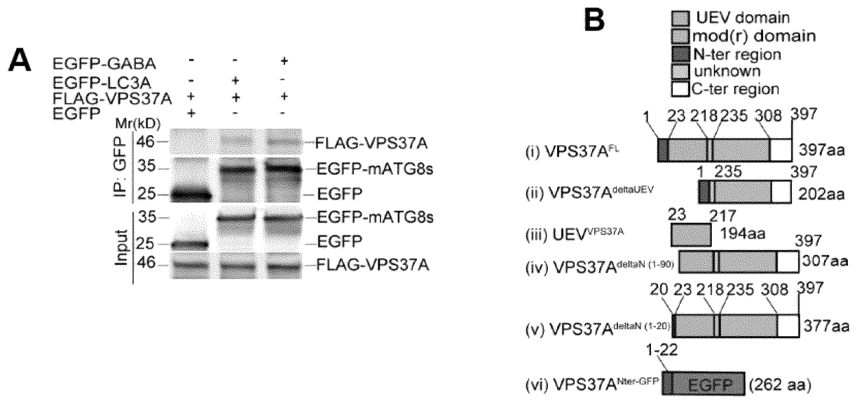

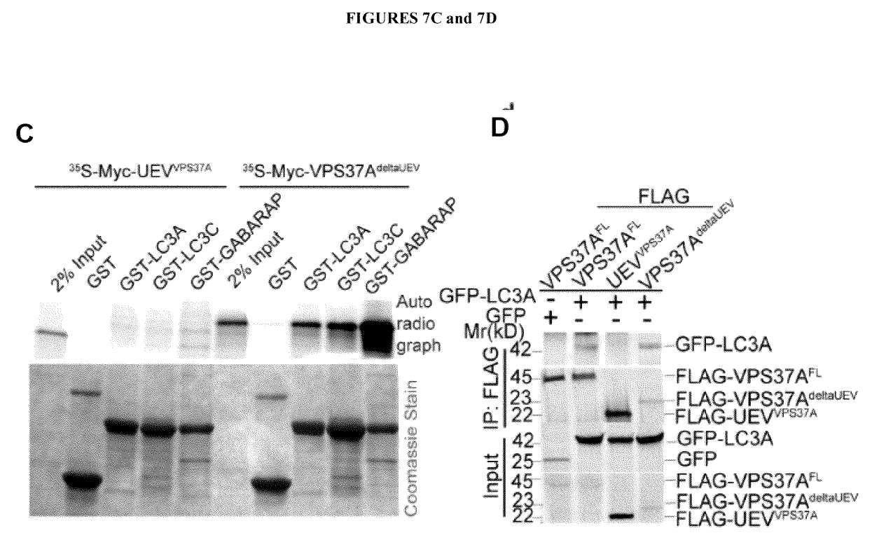

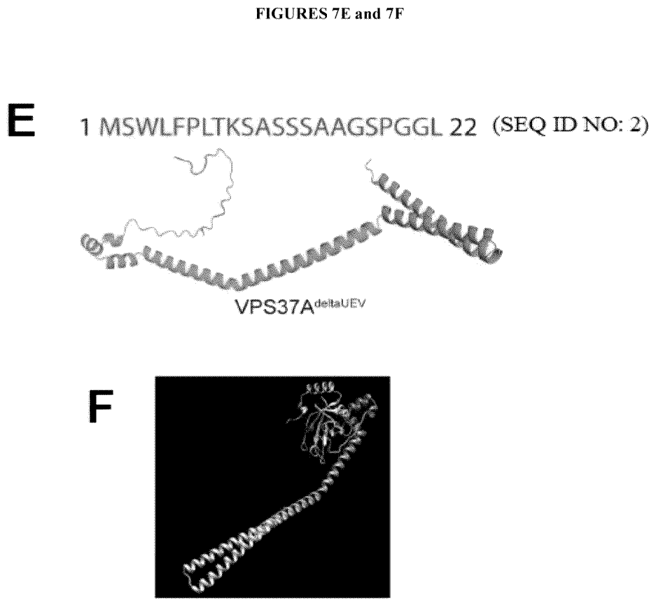

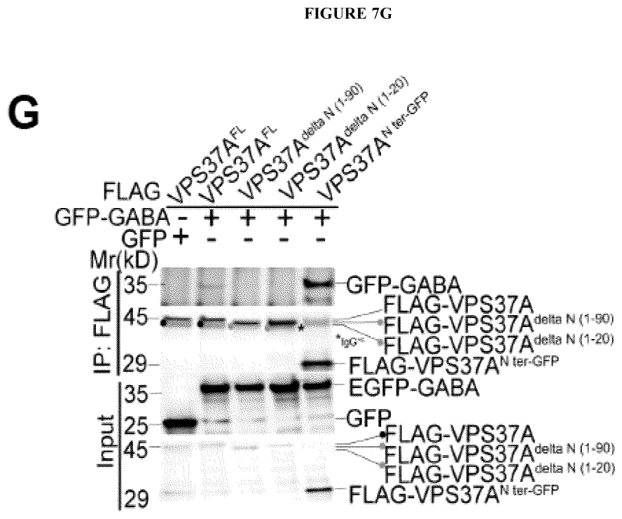

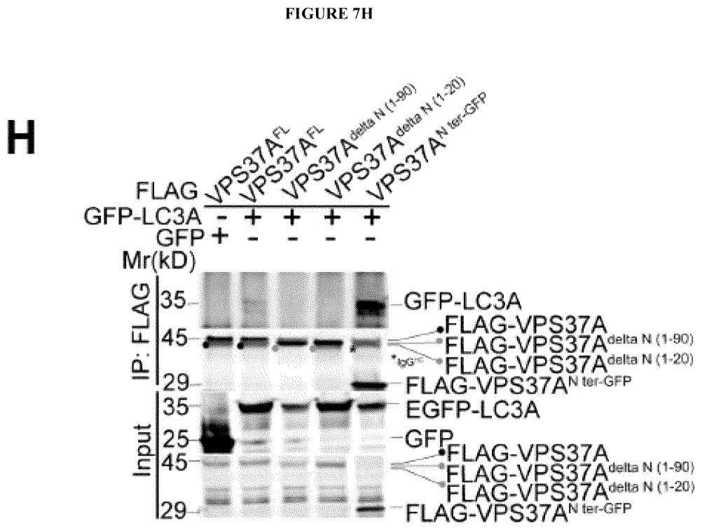

A component of the ESCRT-I complex, VPS37A, has been proposed to play a key role in autophagosomal closure⁴². VPS37A was identified as being essential⁴² for autophagosomes to become sealed and inaccessible to small membrane-impermeant ligands labeling a HaloTag reporter⁷¹ fused to LC3B (HT-LC3B)^{41,45}. VPS37A encodes a ubiquitin E2 variant (UEV) domain⁷², with the UEV in TSG101 known to bind ubiquitin⁷³. We hypothesized that mATG8s, which are ubiquitin-like molecules^{46, 62}, may interact with VPS37A. We hence tested whether mATG8s interacted with VPS37A. Direct interactions between VPS37A and several mATG8s from both LC3 and GABARAP subgroups were observed in GST pulldowns in vitro, with GABARAP and LC3A as standouts (FIG. 1A). These interactions were confirmed by coimmunoprecipitations in cell extracts (Co-IP) (FIG. 7A) and the details are established in studies below.

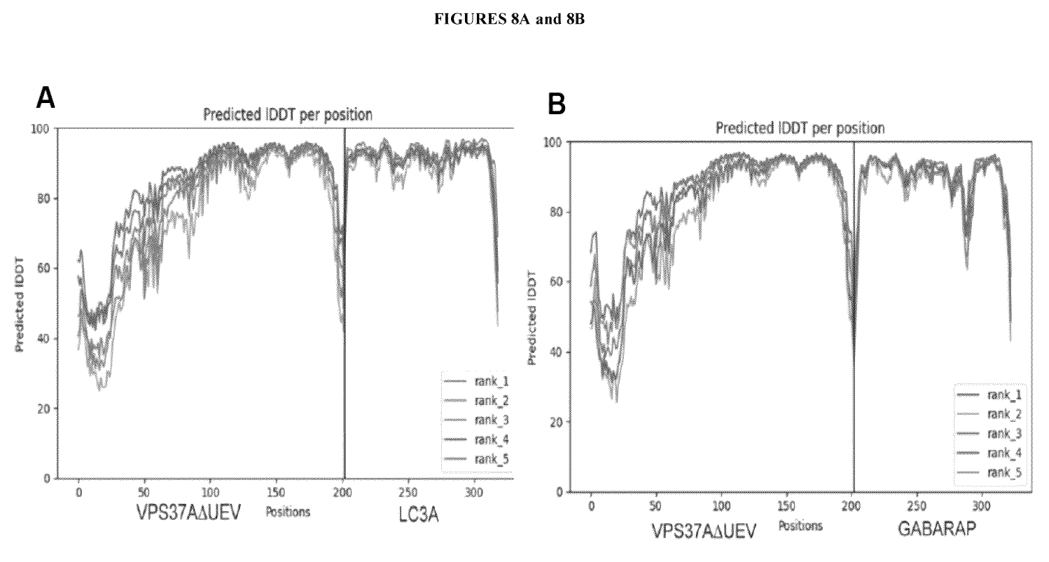

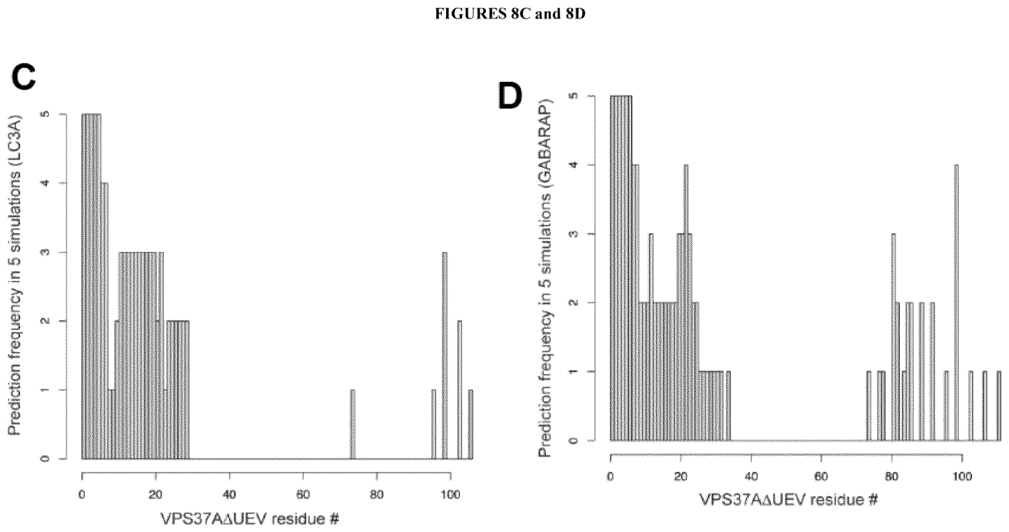

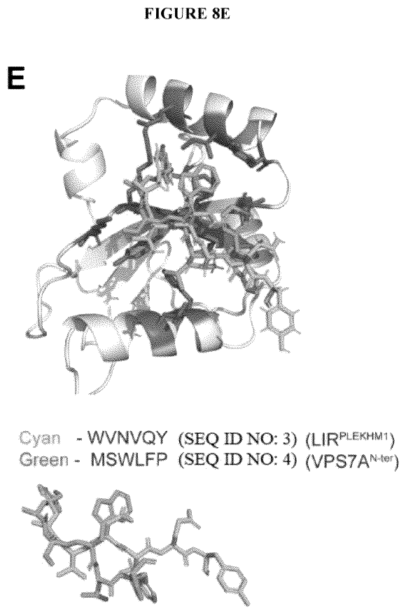

We compared three constructs (FIG. 7B, top): GST-VPS37A^{FL} (full length, FIG. 7B i), GST-VPS37A^{deltaCEV} (UEV domain deleted from the construct, FIG. 7B ii), and GST-UEV^{VPS37A} (only the UEV domain, FIG. 7B iii), with UEV being defined per the NCBI entry NP_689628.2. Unexpectedly, the GST-UEV^{VPS37A} protein did not associate with mATG8s whereas GST-VPS37A^{deltaCEV} did (FIG. 7C), a surprising finding that was confirmed in Co-IP with LC3A (FIG. 7D). Previous work 42 has presented evidence and concluded that the UEV domain of VPS37A was essential for autophagosomal closure. However, the constructs used in that study (VPS37A^{delta1-90}; also known as VPS37A variant 4, FIG. 7B iv) had in addition to the missing portion of the UEV domain a deletion of the entire N-terminal region (residues 1-22; red in FIG. 7B)⁴². Using AlphaFold structure prediction for UniProt entry Q8NEZ2 (https://alphafold.ebi.ac.uk/entry/Q8NEZ2), we modeled interactions between mATG8s and VPS37A (using VPS37A^{deltaCEV} for reduced complexity; FIG. 1B FIGS. 7E, 7F and FIGS. 8A-8D). AlphaFold modeling predicted that the N-terminal region of VPS37A was disordered and that it interacted with mATG8s (FIG. 7E) with additional potential contacts elsewhere in VPS37A. We focused on the N-terminal region. Five independent iterative runs by AlphaFold yielded similar models ranked based on local Difference Distance Test confidence measure (FIGS. 8A,8B). In all 5 iterative models with either LC3A or GABARAP, the N-terminal residues of VPS37AdeltaCEV were consistently predicted to interact with mATG8s (FIGS. 8C,8D). The rank 1 model was chosen as the representative model due to its highest confidence score for the N-terminus region of VPS37A^{deltaCEV}. It showed a predicted sidechain packing albeit with differences within the hydrophobic pocket of GABARAP and LC3A when compared to the known structure of LC3C in a complex with the PLEKHMI LIR peptide (PDB DOI: 10.2210/pdb5DPW/pdb)⁷⁴ (FIG. 8E, cyan PLEKHMI LIR; green N-Ter VPS37A) Thus, AlphaFold predicts at least one binding site (the N-terminal region) within VPS37A^{deltaCEV} for association with GABARAP and LC3A (FIG. 7E,7F and FIG. 1B), albeit additional contacts elsewhere in the molecule are possible.

We next tested by Co-IPs whether the N-terminus of VPS37A binds to GABARAP and LC3A as predicted by AlphaFold. A deletion of the residues 1-90 (FIG. 7B, iv), which has been reported to abrogate VPS37A ability to guide autophagosomal closure⁴², resulted in a loss of interactions with GABARAP and LC3A expressed as GFP fusions (FIG. 7G,7H). An N-terminal deletion of the first 20 residues alone of VPS37A (FIG. 7B, v) prevented detection of interactions by Co-IP (FIGS. 7G,7H). Lastly, the N-terminus of VPS37A (aa 1-22, including a Gly-Gly motif after the first 20 residues; FIG. 7E) when fused to the N-terminus of GFP (FIG. 7B, vi) and FLAG at N terminus of VPS37A to perform Co-IP, was able to bind GFP-LC3A and GFP-GABARAP. The inventors thus conclude that VPS37A's region, implicated in autophagosomal closure⁴², associates with LC3A and GABARAP.

### Mammalian ATG8 Proteins are Necessary for Sealing of Autophagic Membranes

To test the role of mATG8s in autophagosome closure, we used the previously described strategy⁴¹ employing stable cell lines expressing HT-LC3B, used to assess autophagosomal closure^{41, 42, 45}. We adapted this assay for high content microscopy (HCM) to allow unbiased operator-independent image collection, object identification and quantification, as previously established^{30, 75} (FIG. 1C). For this, isogenic cell lines, parental HeLa^{WT} and its derivative HeLa^{HexaKO} with inactivated 6 mATG8s (LC3A,B,C, and GABARAP, L1, L2; HeLa^{HexaKO}) were modified to stably express HT-LC3B. Cells were seeded in 96 well plates, starved for 90 min in EBSS to induce autophagy, subjected to selective plasma membrane permeabilization, and sequentially stained with fluorescent HaloTag ligands that covalently modify haloalkane dehalogenase, as described⁴¹. First, the membrane impermeant ligand (MIL), a compound with haloalkane dehalogenase-reactive linker and fluorescent reporter Alexa Fluor 660 fluorescing at 690 nm (pseudocolored as green), was applied to serve as a reporter for unsealed autophagosomes⁴¹. The initial exposure to the MIL ligand saturates all accessible haloalkane dehalogenase HT-LC3B molecules except those that are inaccessible by being sequestered away from the cytosol, such as the ones enclosed within autophagosomes⁴¹. The HT-LC3B molecules that remained inaccessible to MIL within sealed autophagosomal or sequestered in other endomembranes are then revealed by staining with membrane permeant ligand (MPL), a compound containing haloalkane dehalogenase-reactive linker with tetramethylrhodamine fluorescing at 585 nm (colored red). The plates were scanned to quantify MPL⁺ (sealed) and MIL⁺ (unsealed) autophagosomes and any additional LC3B⁺ structures, and data (quantitative HCM) presented as the number of MIL⁺ or MPL⁺ profiles/cell and their ratios (MIL/MPL) (FIG. 1D). MIL⁺MPL⁺ profiles were also observed but represented a very small fraction (<5%) and were not differentiated in quantifications. Both MIL⁺ and MPL⁺ profiles increased with starvation in HeLa^{WT} HT-LC3B cells, whereas MPL⁺ but not the MIL⁺ profiles were further elevated in starved cells treated with bafilomycin A1 (BafA1) (FIG. 1D, subpanels i and ii), consistent with them representing unsealed and sealed LC3B⁺ membranes including autophagosomes. When compared to HeLa^{WT} HT-LC3B cells, HeLa^{HexaKO} HT-LC3B cells showed a further increase in MIL⁺ profiles with a decrease in MPL⁺ profiles (FIG. 1D, subpanels i and ii), also reflected in elevated ratios of MIL vs MPL profile numbers/cell in HeLa^{HexaKO} vs HeLa^{WT} cells (FIG. 1D, subpanel iii). The diminished numbers of MPL⁺ profiles in HeLa^{HexaKO} HT-LC3B cells relative to HeLa^{WT} HT-LC3B cells could not be enhanced by BafA1 treatment, excluding an increase in autophagic turn-over as a potential explanation (FIG. 1D, subpanel ii). Of note, HT-LC3B does not complement this phenotype in HeLa cells devoid of the six principal mATG8s (HeLa^{HexaKO})⁶⁹, indicative of the observed effect being independent of LC3B. These findings, taken together with the prior interpretations of the HT-LC3B-MIL/MPL assay⁴² suggest that mATG8s participate in membrane sealing of autophagosomal organelles.

Ultrastructural Analysis of Organelles in Cells Devoid of the Principal mATG8s

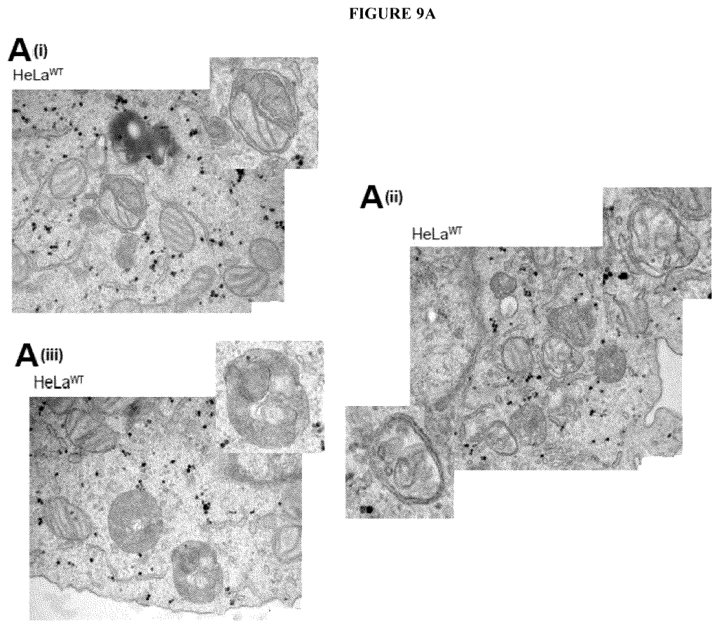

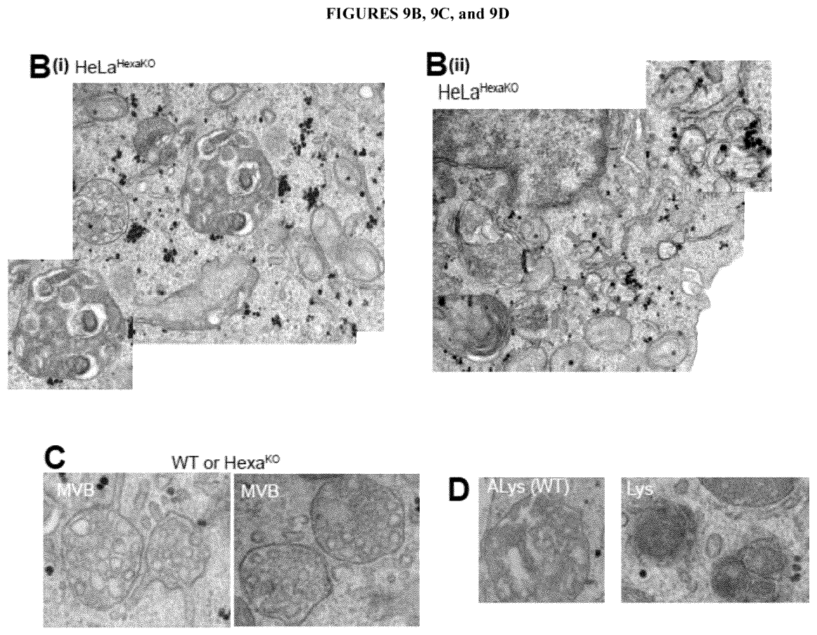

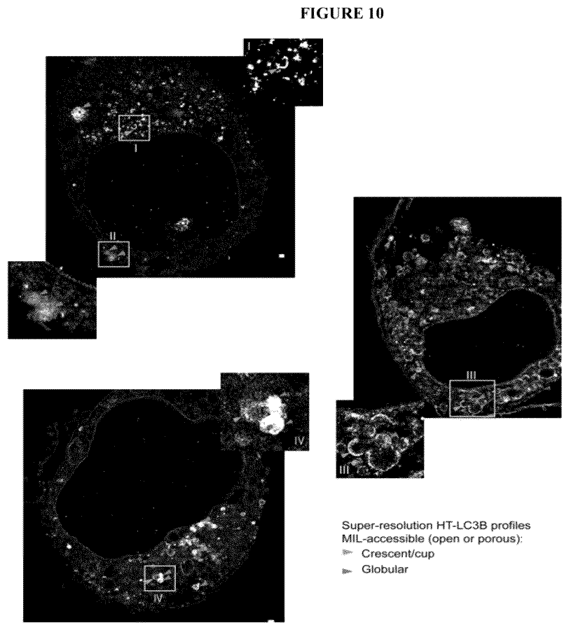

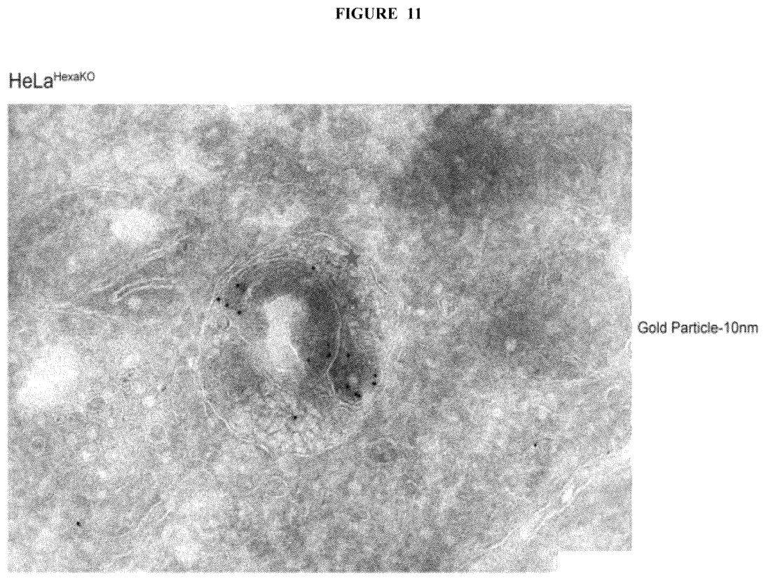

Given that the MIL/MPL assay data suggest that autophagosomes are not fully sealed in the absence of mATG8s, we carried out electron microscopy (EM), immuno-EM and super-resolution (SR) analyses to morphologically examine autophagic organelles. The principal findings of the EM studies (FIGS. 1E-G and FIGS. 7A-7D) indicated that in the absence of mATG8s in HeLa^{HexaKO} there was an accumulation of organelles morphologically identified as amphisomes, whereas there was a reduction in more mature, degradative autolysosomes (FIG. 1G). Amphisomes are a product of autophagosomal fusion with early and late endosomes³⁷. This includes MVBs, which contain intraluminal 40-60 nm vesicles and are typical fusion partners in amphisome formation³⁸⁻⁴⁰. A simultaneous increase in amphisomes with reduction in autolysosomes (FIG. 1G) suggests a precursor relationship for amphisome vis-à-vis autolysosomes, and indicates that in the absence of mATG8s the autophagic intermediates are arrested at the amphisomal stage. Amphisomal morphology was consistent with previously reported images of autophagic structures accumulating in HeLa^{HexaKO} and not maturing into autolysosomes⁶⁹. An increase in amphisomes could not be explained by a possible increase in MVBs in HeLa^{HexaKO} cells, as these remained similar in HeLa^{HexaKO} and HeLa^{WT} cells (FIG. 1G and FIGS. 9C,9D). By SR dSTORM analyses, we observed in HeLa^{HexaKO} HT-LC3B cells a variety of profiles including phagophores and apparently closed vesicular/globular structures that were not sealed (FIG. 1H and FIG. 10) since they remained permeant to small molecules and were stained with MIL. By immuno-EM of HeLa^{HexaKO} transfected with GFP-LC3A, we observed immunogold labeling of GFP inside the amphisome-like structures (FIG. 11). Thus, we conclude that whereas autophagic organelles progress to the morphological stage known as amphisomes, the fine integrity of their membranes, reflected in permeability to small molecules exchanging with the cytosol, is compromised in the absence of mATG8s.

In Vitro System for Membrane Sealing Confirms the Role of mATG8s

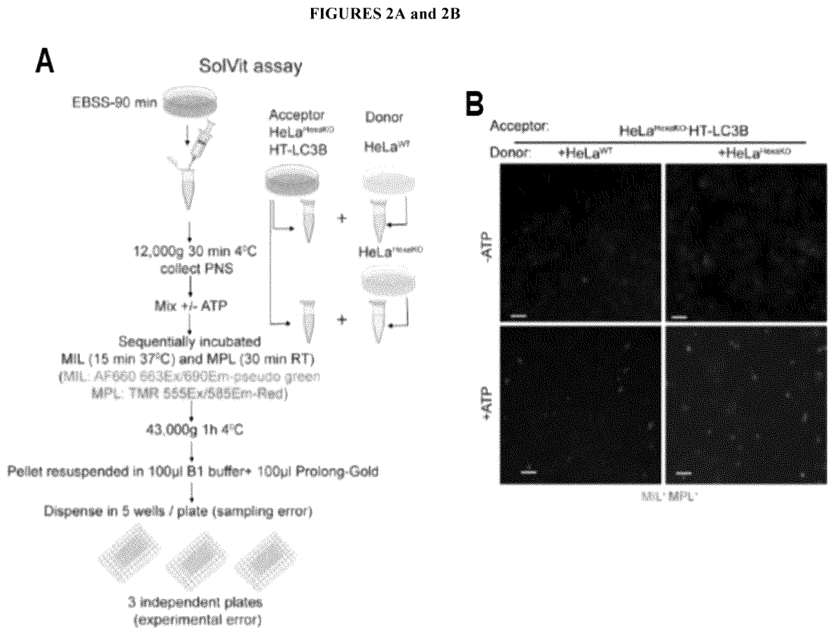

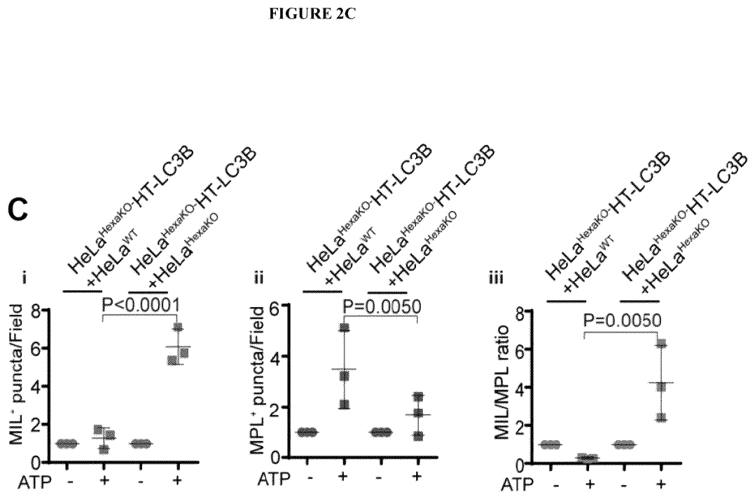

To further establish that mATG8s play a role in sealing of autophagosomal membranes we developed an in vitro assay termed SolVit (sealing of organellar limiting membranes in vitro) utilizing the HT-LC3B and MIL/MPL system as a reporter (FIG. 2A). For SolVit, postnuclear supernatants (PNS) prepared by gentle sheer lysis of HeLa^{HexaKO} cells stably expressing HT-LC3B (Acceptor) are combined with PNS from either HeLa^{HexaKO} or HeLa^{WT} lysates (Donor) in the presence or absence of ATP, followed by sequential staining with MIL and MPL. The resulting reaction products are then mounted in 96 well plates and subjected to HCM quantification of MPL⁺ (red) and MIL⁺ (green) profiles in up to 60 different fields until a preset limit of 1,000 objects is reached (fields of view are illustrated in FIG. 2B). Membrane sealing in vitro in ATP-dependent reactions, i.e. HT-LC3B sequestration and protection from covalent saturation by MIL, allows subsequent staining with MPL, (FIG. 2C). Addition of HeLa^{HexaKO} as donor PNS to the HeLa^{HexaKO} HT-LC3B acceptor PNS, yielded only an increase in MIL⁺ (unsealed) profiles and did not increase MPL⁺ (sealed) profiles (FIG. 2C-i,ii). Addition of HeLa^{WT} PNS as donor to HeLa^{HexaKO} HT-LC3B as acceptor PNS, resulted in increased MPL⁺ (sealed) profiles (FIG. 2C-ii), also reflected in the MIL⁺/MPL⁺ ratios (FIG. 2C-iii). Thus, mATG8s are important for sealing of LC3⁺ positive organelles in vitro, confirming findings with whole-cell HCM studies.

Both GABARAP and LC3 Subsets of mATG8s Play a Role in Membrane Sealing

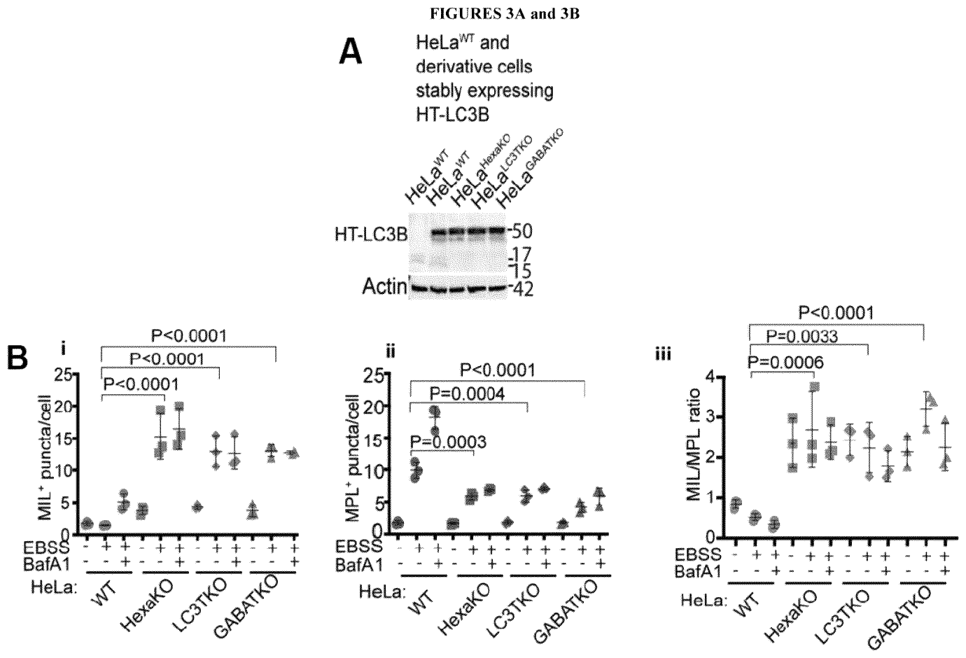

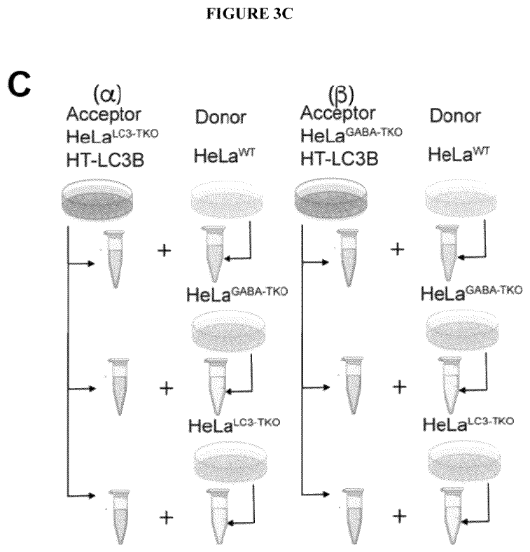

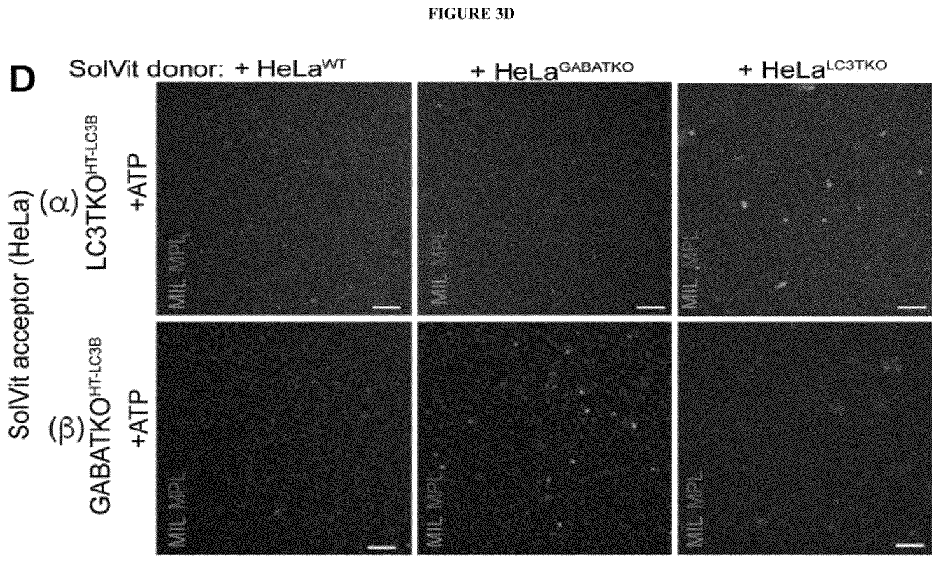

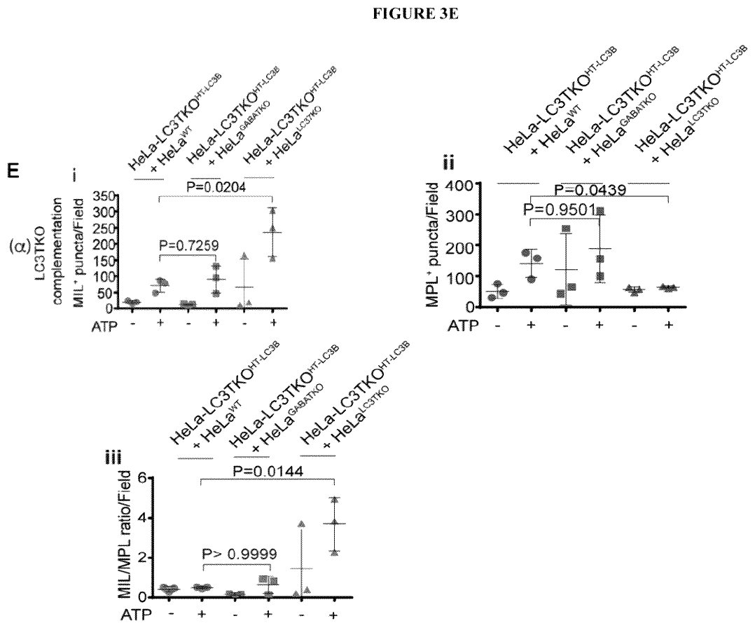

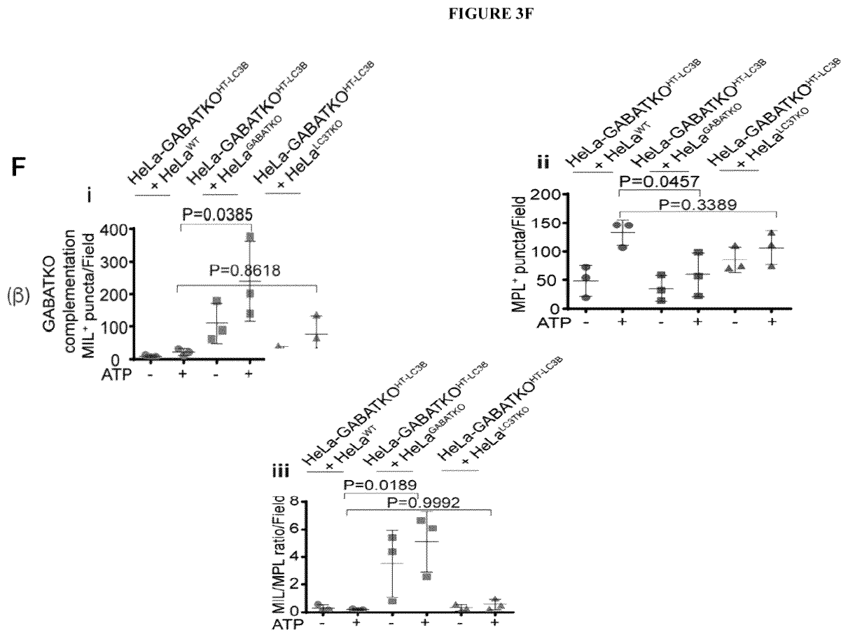

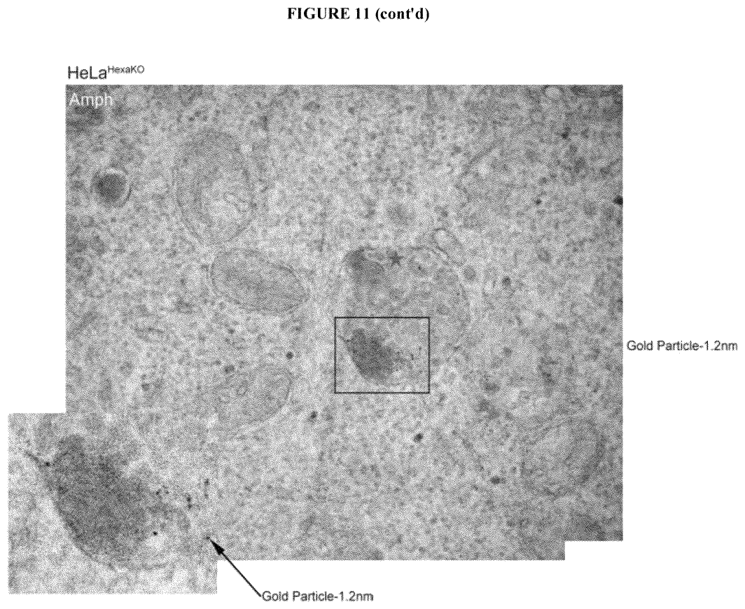

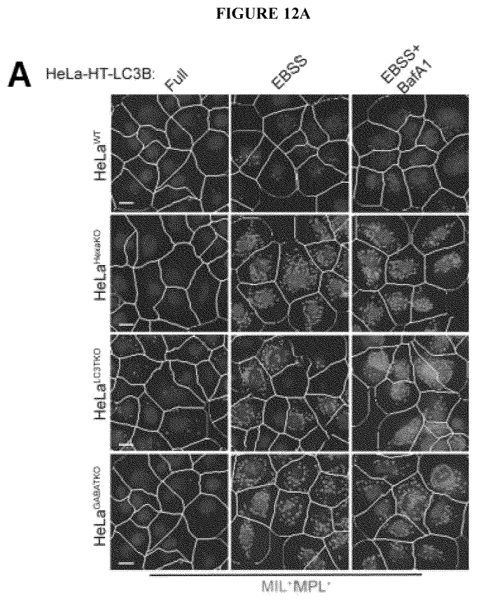

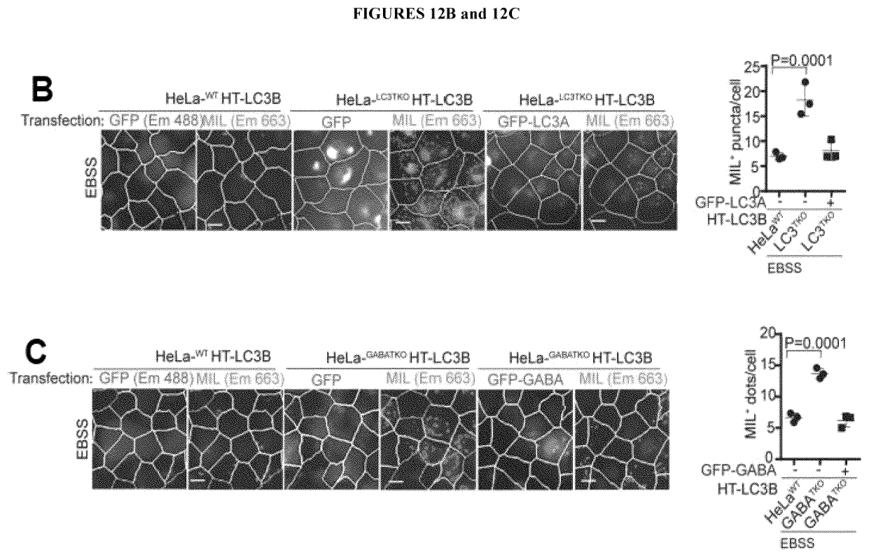

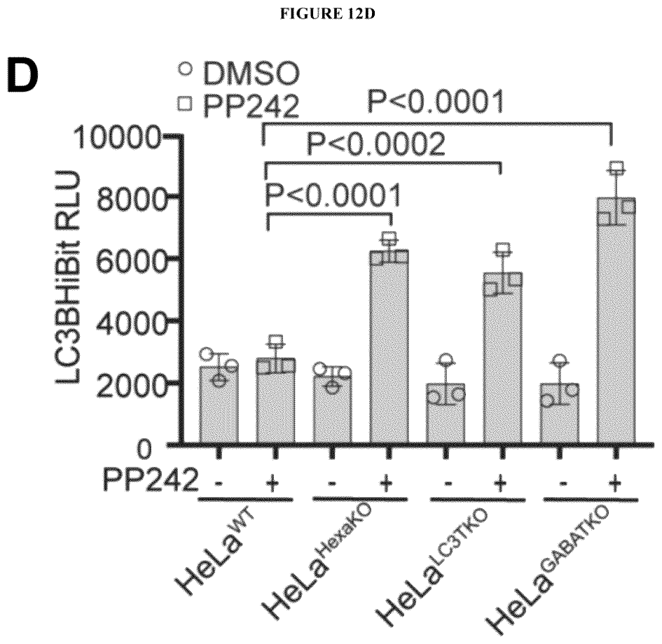

The GST pulldown experiments (FIG. 1A) indicate that members of both subgroups of mATG8s, GABARAPs and LC3s, can bind VPS37A, with a notable exception of LC3B. We thus tested whether both subfamilies, GABARPs and LC3s, play a role in keeping the LC3B⁺ profiles sealed. We derived HeLa lines stably expressing HT-LC3B (FIG. 3A) in parental cells inactivated for three LC3s (LC3A,B,C; HeLa^{LC3TKO}) and three GABARAPs (GABARAP, L1, L2; HeLa^{GABATKO})⁶⁹, and subjected them to HCM analysis with the MIL/MPL staining of cells induced for autophagy by starvation in EBSS (FIG. 3B and FIG. 12A). Both HeLa^{LC3TKO} and HeLa^{GABATKO} cells displayed an increase in unsealed (MIL⁺) HT-LC3B profiles and a decrease in sealed (MPL⁺) profiles relative to HeLa^{WT}, similarly to HeLa^{HexaKO} (FIG. 3B). As a further control we treated cells induced for autophagy with BafA1 to block progression to degradative autolysosomes. As expected, EBSS induced MPL⁺ profiles in HeLa^{WT}, further increased in the presence of BafA1 (FIG. 3B). The increase in MPL⁺ profiles with EBSS was diminished in HeLa^{HexaKO}, HeLa^{LC3TKO}, and HeLa^{GABATKO} cells and there was no detectable further MPL⁺ increase in the presence of BafA1 (FIG. 3B). We transfected HeLa^{LC3TKO} with GFP-LC3A and HeLa^{GABATKO} cells with GFP-GABARAP and observed complementation by decreased MIL⁺ staining in cells expressing GFP-mATG8s fusion constructs (MPL could not be assessed due to fluorescence overlap between TMR and GFP) (FIGS. 12B, 12C).

We additionally employed an independent, modified split luciferase-based assay to assess the accessibility of LC3B (FIG. 12D). In the split-luciferase assay, NanoLuc holoenzyme is split into N-terminal HiBit 1.3-kDa domain and 18-kDa LgBit C-ter domain⁷⁶. Cells (HeLa^{WT}, HeLa^{HexaKO}, HeLa^{LC3TKO} and HeLa^{GABATKO}) were transfected with the HiBit-LC3B expressing plasmid. They were then treated with the mTOR inhibitor PP242 to induce autophagy. Cells were subjected to plasma membrane permeabilization (using treatment as in the MIL/MPL assay), after which LgBit was added along with the luciferase substrate furimazine in a non-lytic (membrane non-permeabilizing) buffer and luminescence quantified. Higher luciferase activity was observed in HeLa^{HexaKO}, HeLa^{LC3TKO} and HeLa^{GABATKO} relative to HeLa^{WT} (FIG. 12D). This was indicative of increased access of LgBit to HiBit-LC3B localized within unsealed membranes.

Subsets of mATG8s Contribute to Sealing of LC3B Membranes In Vitro

To confirm that both LC3s and GABARAPs participate in maintaining autophagic membranes sealed, we performed in vitro complementation assays in the SolVit system (FIG. 3C-F). For this, the acceptor PNS was from either HeLa^{LC3TKO} (FIG. 3Ca) or HeLa^{GABATKO} (FIG. 3Cβ) stably expressing HT-LC3B, combined with donor PNS preparations from HeLa^{WT}, HeLa^{LC3TKO} or HeLa^{GABATKO}. As a control, ATP was either added or not to the incubation mixture of PNS extracts. After incubation, the ATP-dependent products of the in vitro reaction were subjected to HCM quantification. When the acceptor was either HeLa^{LC3TKO} HT-LC3B or HeLa^{GABATKO} HT-LC3B, addition of cross-complementing PNS from HeLa^{WT} cells resulted in sealing of LC3B⁺ profiles in the presence of ATP reflected in increased MPL⁺ objects (FIGS. 3E,F, middle panels/red). A similar increase in MPL⁺ profiles was observed when heterologous PNS extracts were combined (HeLa^{LC3TKO} HT-LC3B×HeLa^{GABATKO} or HeLa^{GABATKO} HT-LC3B×HeLa^{LC3TKO}) but not when homologous PNS combinations were used (HeLa^{LC3TKO} HT-LC3B×HeLa^{LC3TKO} or HeLa^{GABATKO} HT-LC3B×HeLa^{GABATKO}) (FIGS. 3E,F). This was reflected in other parameters, e.g., MIL increased in products of homologous and decreased in products of heterologous reaction mixtures (FIGS. 3E,F panels i). Finally, MIL/MPL ratios increased in products of homologous and decreased in products of heterologous reaction mixtures (FIGS. 3E,F panels iii). Thus, both GABARAP and LC3 members contribute to sealing of LC3B⁺ membranes and maintaining them impermeant to small molecules.

### ATG9A is Required for Efficient Sealing of Autophagosomal Membranes

The role of ATG9A in autophagy has been recently linked to autophagosomal membrane expansion whereby it acts as a lipid scramblase³³⁻³⁵ relaxing lipid asymmetry between membrane leaflets during ATG2-dependent lipid transfer to autophagosomes^{32-34, 77, 78}. ATG9A has been shown to have additional roles in membrane dynamics, including lamellipodia expansion during cell migration⁷⁹ and plasma membrane repair with the latter occurring through its interactions and cooperation with ESCRTs⁸⁰. Thus, we hypothesized that ATG9A may contribute to ESCRT-dependent sealing of autophagosomes.

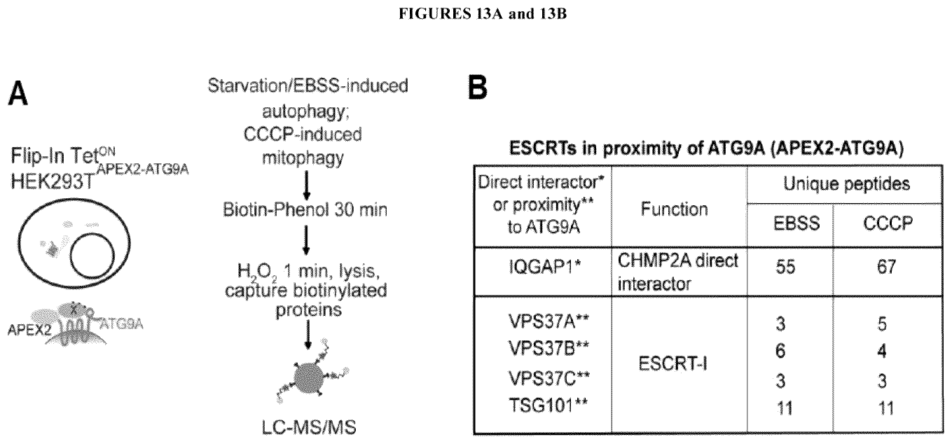

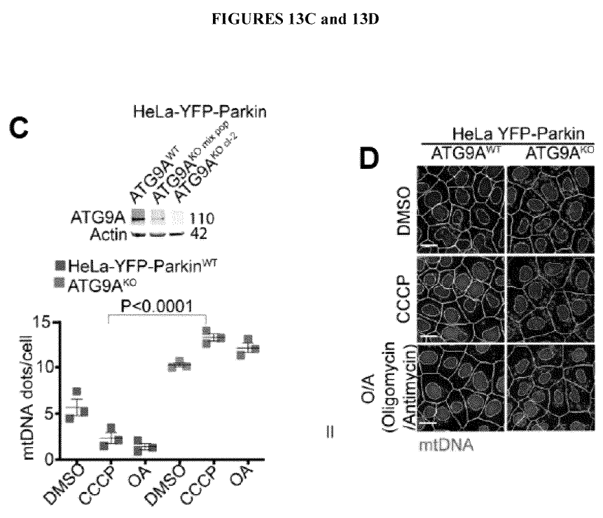

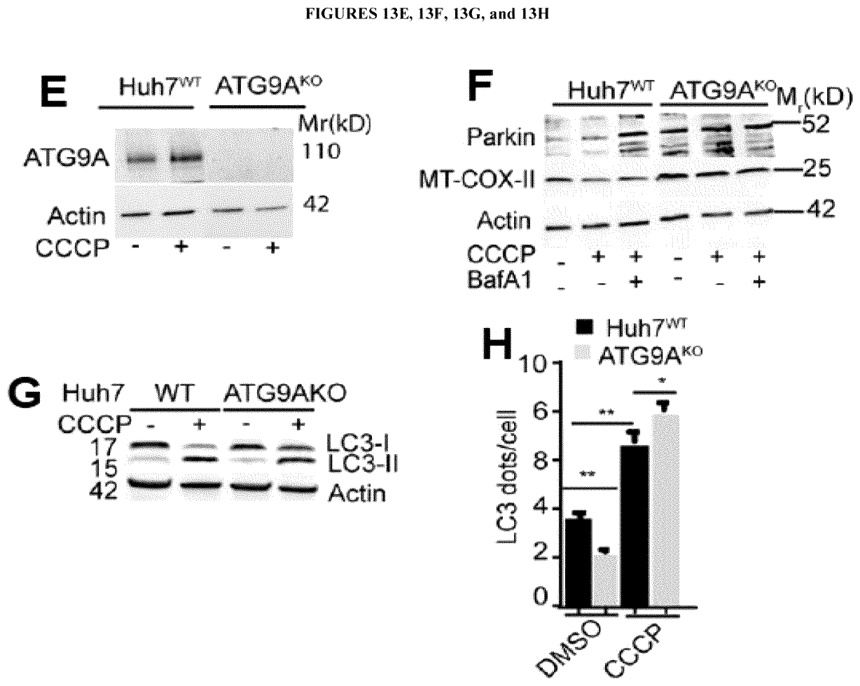

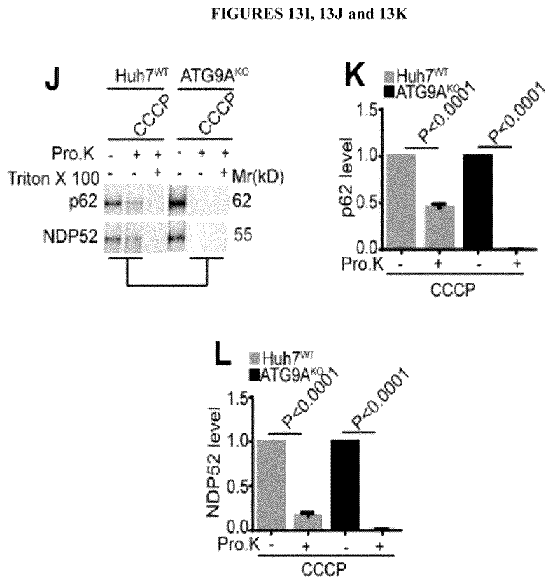

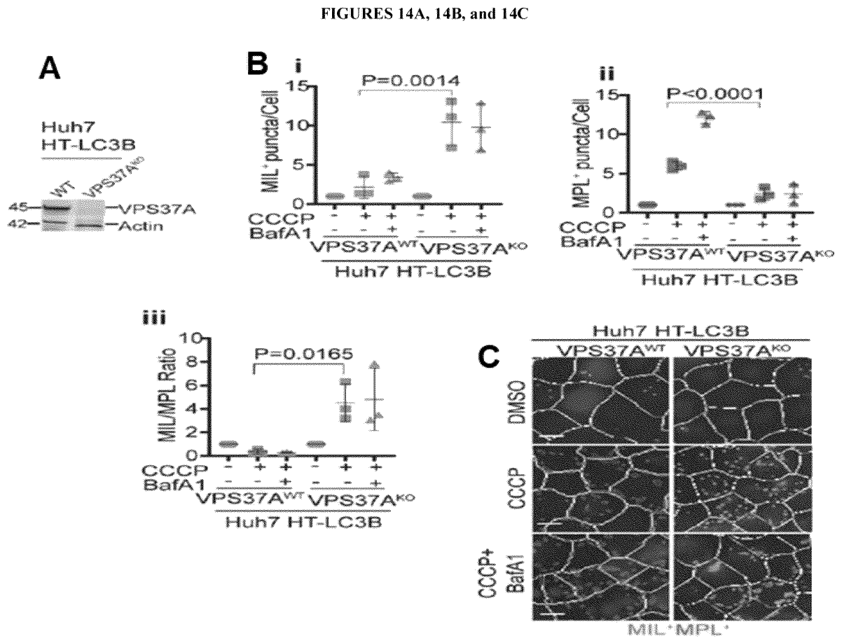

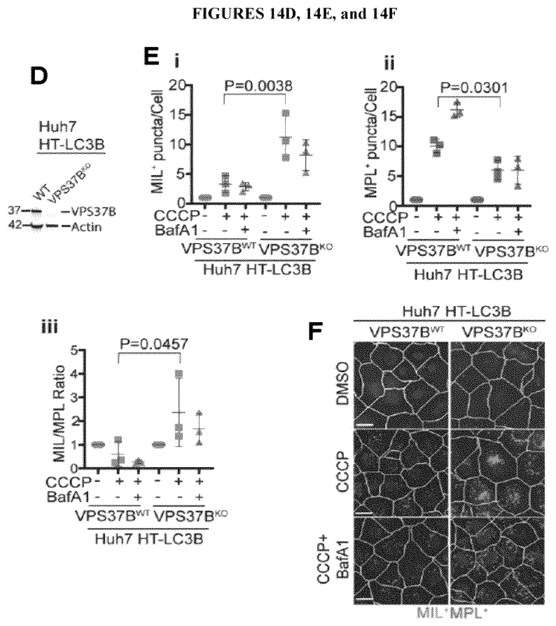

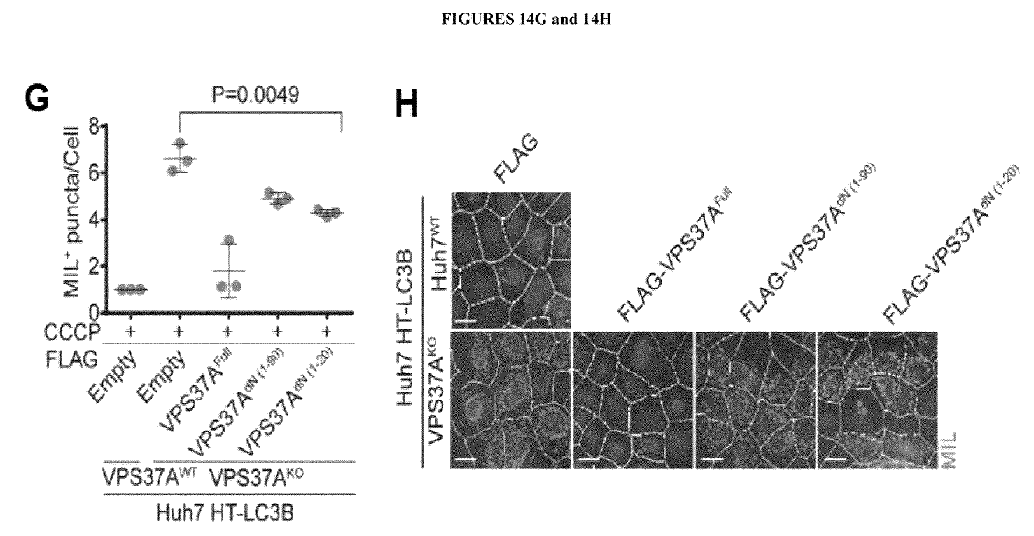

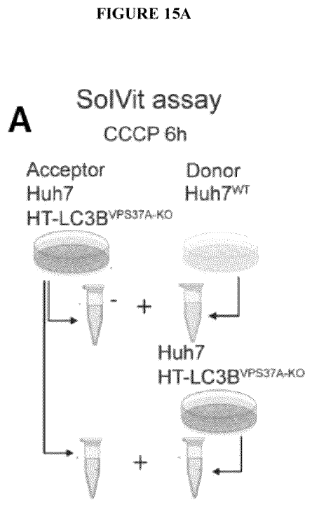

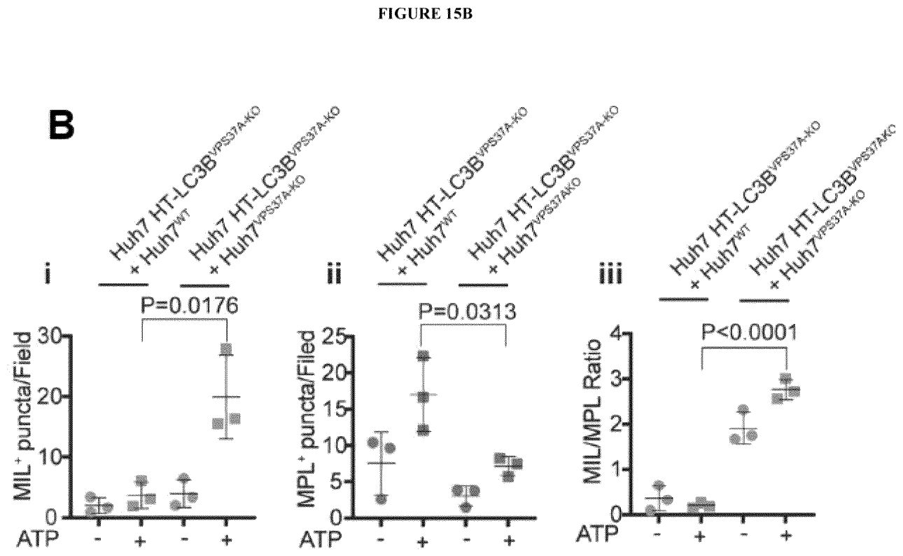

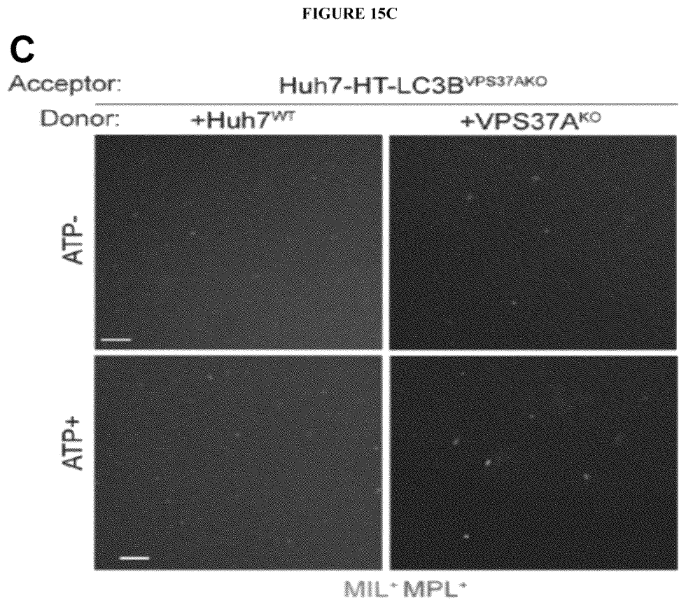

Using proximity biotinylation proteomics, we experimentally established that ATG9A engaged ESCRTs during starvation-induced autophagy or CCCP-induced mitophagy (FIG. 13A,13B). During CCCP-induced mitophagy, ATG9A inactivation does not affect LC3B lipidation, as previously reported⁸¹. ATG9A's scramblase activity is necessary for expansion of prophagophores to generate normal size double membrane autophagosomes but is not necessary for their closure by conventional EM analysis³⁴. Although ATG9A was needed for mitophagy in HeLa cells stably expressing YFP-Parkin^{69, 82} (FIGS. 13C,13D), we could not use HeLa cells expressing YFP-Parkin in the MIL/MPL assay due to the overlap between YFP-Parkin fluorescence and MPL. We thus employed Huh7 cells (FIG. 13E), which express endogenous Parkin (FIG. 13F). In Huh7 cells, the ESCRT-I components⁴⁵ VPS37A and VPS37B were required for autophagosomal sealing during mitophagy in whole cells (HCM, FIGS. 14A-14F) and in vitro (SolVit, FIG. 15), whereas VPS37A function depended on its mATG8-binding capacity (complementation, FIGS. 14G-14H). The previously characterized ATG9A knockout in Huh7 cells⁸⁰ affected CCCP-induced degradation of mitochondrial COX-1I (FIG. 12F), whereas LC3B puncta formation and LC3B lipidation were not affected by the loss of ATG9A (FIGS. 13G-13H) in keeping with prior observations⁸¹.

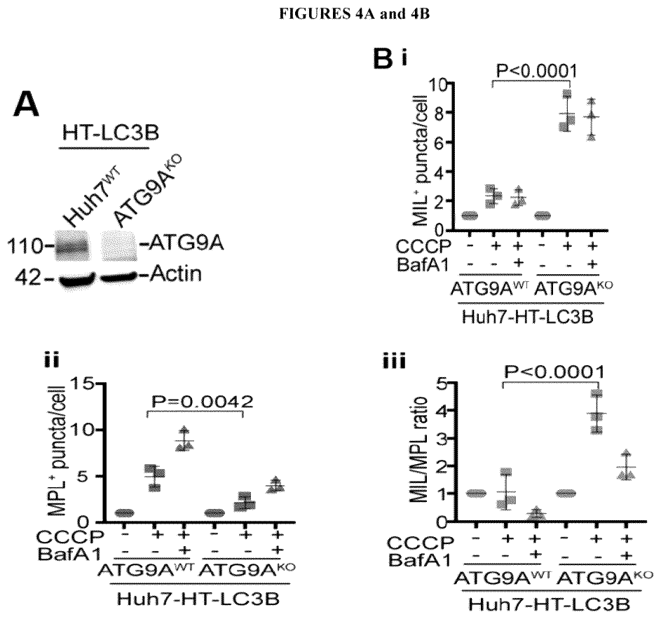

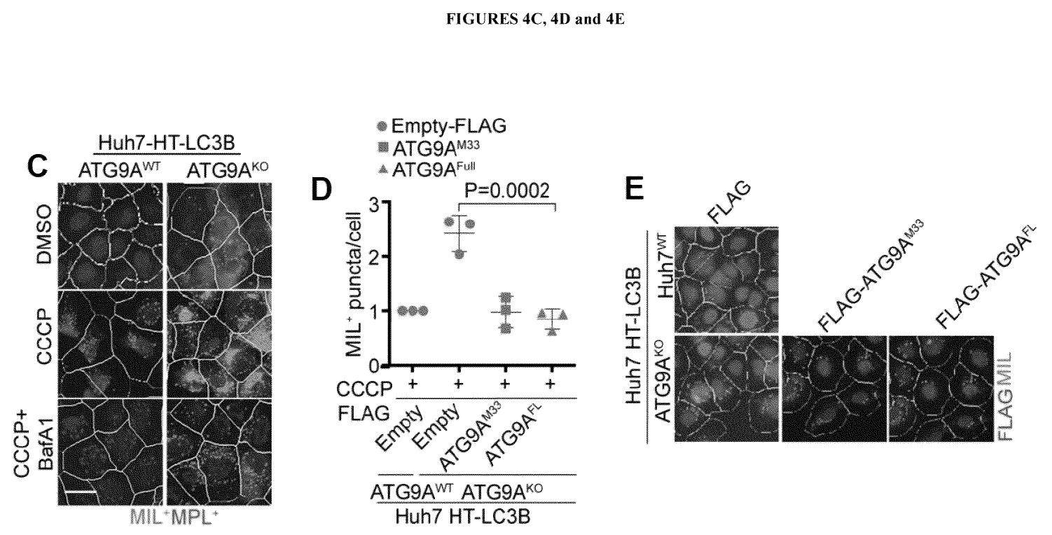

We tested whether ATG9A matters in autophagosomal sealing. For the MIL/MPL assay, we knocked out ATG9A in Huh7 HT-LC3B cells (Huh7 HT-LC3B^{ATG9AKO}) (FIG. 4A) and quantified MIL and MPL puncta induced for mitophagy by CCCP in ATG9A^{WT} and ATG9^{KO} backgrounds (FIGS. 4B,C). ATG9A knockout increased MIL⁺ (unsealed) profiles (FIG. 4B-I), decreased MPL⁺ (sealed) profiles (FIG. 4B-II), and elevated MIL⁺/MPL⁺ ratios (FIG. 4B-III) in cells treated with CCCP. A complementation of this phenotype (measured by MIL staining) was achieved by transfecting cells with Flag-ATG9AWT (FIGS. 4D,E). A scramblase mutant FLAG-ATG9A-M33³⁴ also complemented the elevated MIL staining phenotype in Huh7^{ATG9AKO} HT-LC3B (FIGS. 4D,E), indicating that the effects of ATG9A on sealing of autophagosomes cannot be attributed to a defect in membrane expansion. Thus, by the MIL/MPL assay, ATG9A plays a role in autophagosomal sealing during CCCP-induced autophagy.

This was corroborated in a protease protection assay^{69, 83-85} using selective autophagy receptors p62/SQSTM1 and NDP52. In ATG9A^{KO} Huh7 cells treated with CCCP, both p62 and NDP52 were fully accessible to proteinase K, whereas in the parental ATG9A^{WT} Huh7 cells, a fraction of these receptors was protected from access by proteinase K (FIGS. 13I-13K). The accessibility of autophagic receptors to proteinase K is consistent with the accessibility of HT-LC3B to MIL in ATG9A^{KO} cells during mitophagy.

### ATG9A Contributes to Sealing of Autophagic Membranes In Vitro

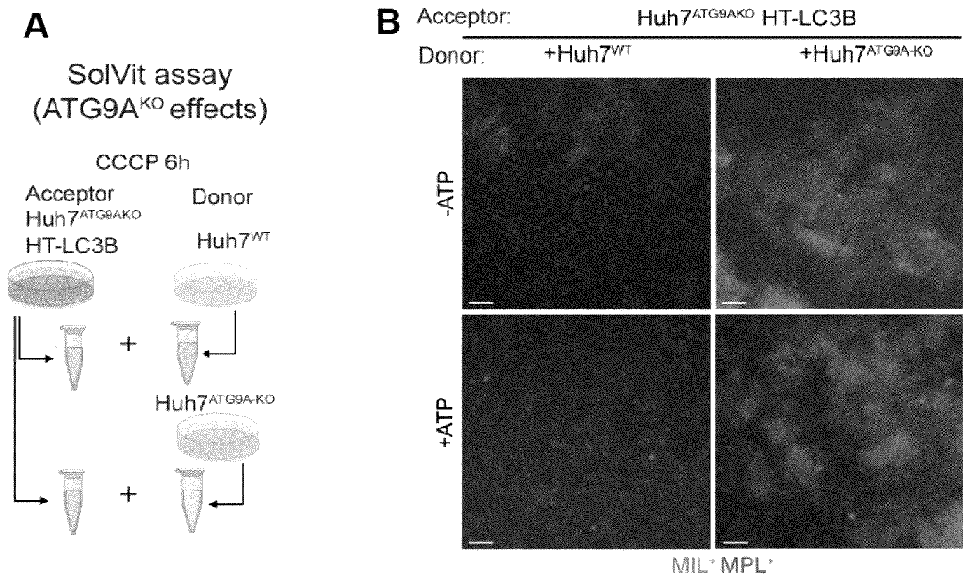

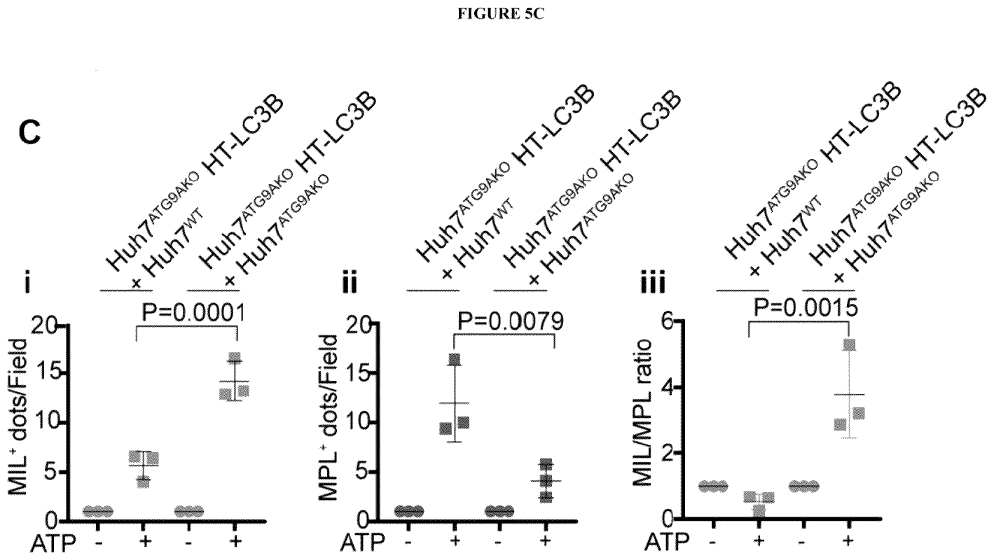

We tested the role of ATG9A in sealing of autophagic membranes in vitro using the SolVit system. Employing Huh7 HT-LC3B^{ATG9AKO} PNS as the acceptor, we added donor PNS from Huh7^{WT} or Huh7^{ATG9AKO} cells and the reaction products were sequentially stained with MIL and MPL (FIGS. 5A,B). MIL, a marker of unclosed/unsealed HT-LC3B membranes, increased on the in vitro products of ATP-dependent reactions when Huh7^{ATG9AKO} PNS was the donor (FIG. 5C-i), whereas MPL, a marker of sealed HT-LC3B membranes, was increased when Huh7WT PNS was added as the donor (FIG. 5C-ii). The MIL/MPL ratio increased in SolVIt assay when Huh7^{ATG9AKO} cells were the source of donor PNS (FIG. 5C-iii). Thus, ATG9A is important for sealing of LC3⁺ positive organelles in vitro, confirming findings with whole-cell HCM studies.

ATG9A Partners with IQGAP1 and CHMP2A to Seal Autophagosomal Membranes

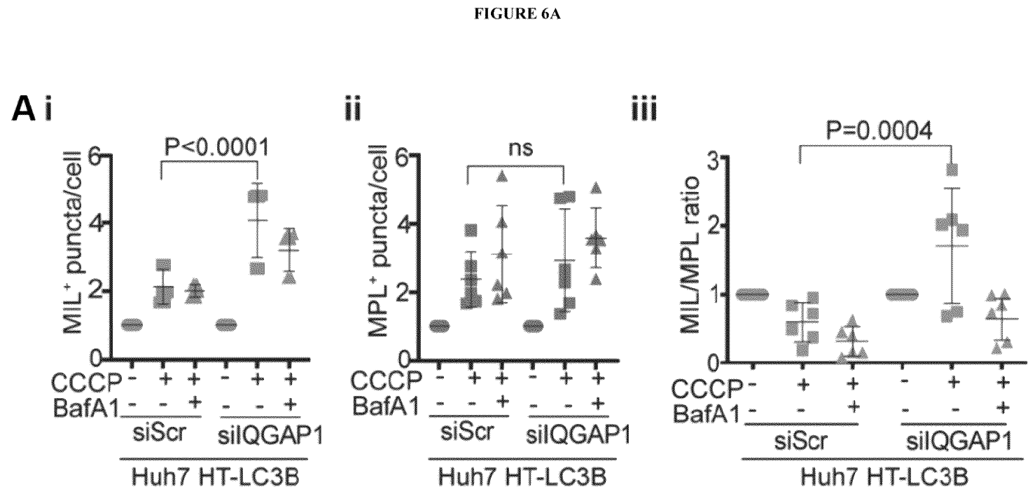

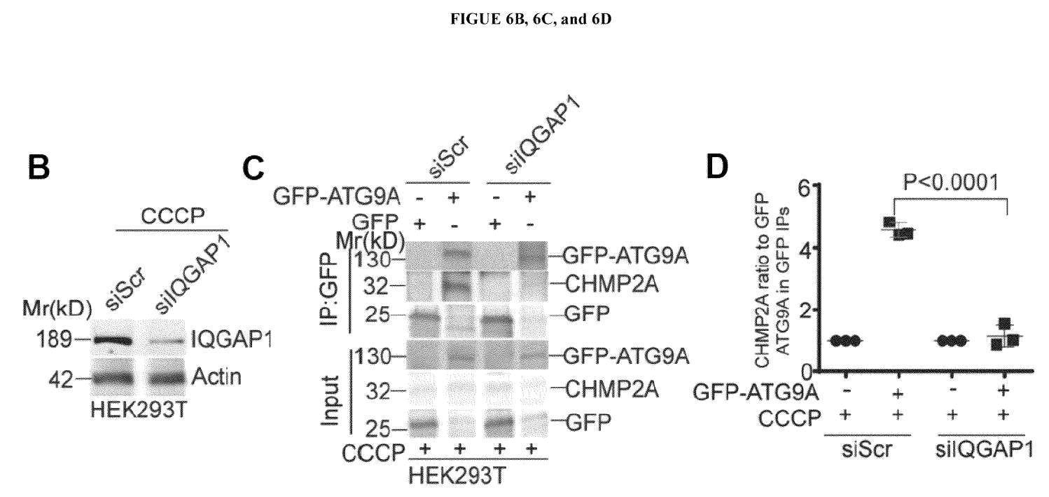

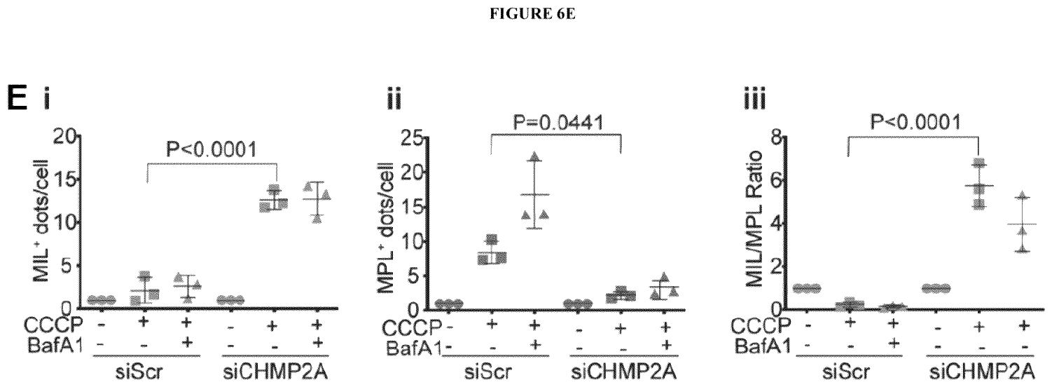

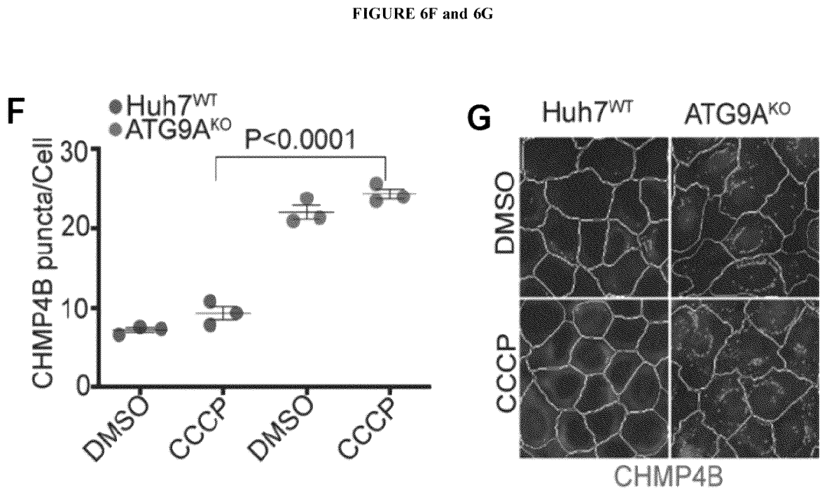

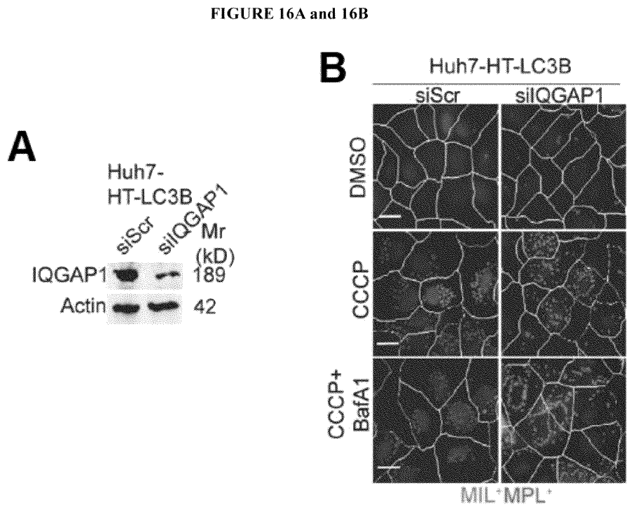

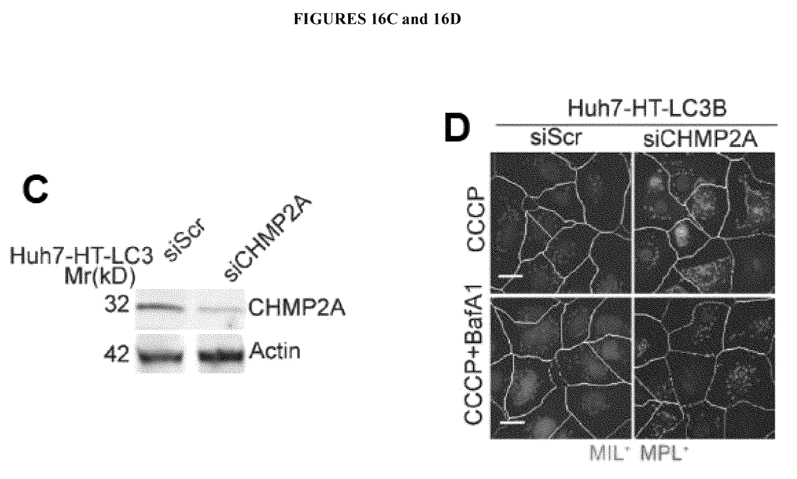

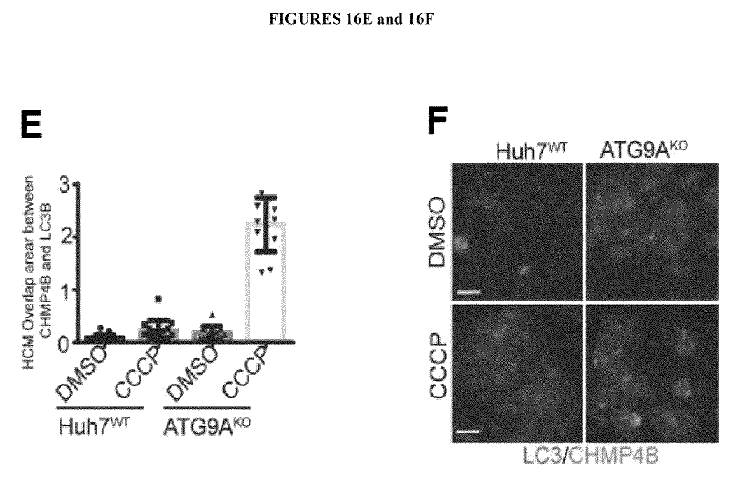

Among the ATG9A partners identified by proteomics was IQGAP1⁸⁶ (FIG. 13B). Although IQGAP1 is not classified as an ESCRT, prior work has shown that it cooperates with ESCRTs⁸⁷ and that it directly interacts with CHMP2A⁸⁰. CHMP2A in turn is key to autophagosomal closure⁴¹ during the final stages of ESCRT action since it is critical for the recruitment of VPS4 to complete the membrane scission and ESCRT filaments disassembly⁴⁴. Thus, we tested whether IQGAP1 contributes to sealing of the autophagosomes. Huh7 HT-LC3B stable cells were knocked down for IQGAP1 (FIG. 16A), mitophagy induced with CCCP, and MIL/MPL staining quantified by HCM (FIG. 6A and FIG. 16B). IQGAP1 knockdown resulted in elevated MIL⁺ profiles and increased MIL⁺/MPL⁺ ratios (FIG. 6A, subpanels i,iii). IQGAP1 was important for association of ATG9A and CHMP2A in immunoprecipitated protein complexes (FIGS. 6B-D). CHMP2A was necessary for efficient sealing of HT-LC3B membranes in cells treated with CCCP, as assessed by the MIL/MPL HCM assay (FIG. 6E and FIGS. 16C,16D). CHMP4B puncta accumulate in cells downregulated for CHMP2A because CHMP2A recruits VPS4 to complete the final stages of membrane scission, as in the absence of CHMP2A the ESCRT-III filaments do not disassemble⁴⁴. We tested whether ATG9A knockout affected CHMP4B puncta during mitophagy. Huh7^{WT} and their derivative Huh7^{ATG9AKO} cells were treated with CCCP and CHMP4B puncta quantified. We found that in ATG9A knockout cells CHMP4B puncta accumulated relative to WT cells (FIGS. 6F,G), indicating that ATG9A phenocopies CHMP2A functions in ESCRT-dependent sealing of membranes. The accumulation of CHMP4B puncta elicited by CCCP and enhanced due to ATG9A knockout occurred on LC3B⁺ membranes (FIGS. 16E,16F). Thus, ATG9A, its interactor and ESCRT-organizing protein IQGAP1, and IQGAP1's partner CHMP2A finalize the sealing of autophagosomal membranes.

### DISCUSSION

The experiments presented herein show that mATG8s and ATG9A orchestrate ESCRT's to maintain autophagosomal membranes in a sealed, nonporous state thus enabling autophagic intermediates to progress into lytic organelles. A model emerges in which mATG8s recruit ESCRT-I component VPS37A, shown to be essential for sealing of the autophagosome⁴². Next, ATG9A brings along its direct interactor IQGAP1⁸⁰. IQGAP1 in turn directly interacts with CHMP2A, essential for the final stages of ESCRT cascade needed to catalyze the membrane scission⁴¹. This is an ATP hydrolysis requiring step catalyzed by VPS4⁴⁴, reflected in the dependance on ATP of our in vitro membrane sealing system. Our data are consistent with a sequential action of mATG8s and ATG9A in autophagic membrane sealing, whereby mATG8s initiate the process via ESCRT-I and ATG9A finishes it via ESCRT-III.

The role of mATG8s in the maintenance of autophagic structures in a sealed state represents their key and previously unappreciated function and expands the portfolio of mATG8s′ roles that include interactions with autophagic cargo receptors⁶³⁻⁶⁵, membrane perturbation during canonical autophagosome formation⁶⁷, a kinetic role in autophagosome-lysosome fusion⁸⁸, and participation in non-canonical processes³.

In the absence of VPS37A, autophagic organelles have been reported to remain at more immature morphological stages, i.e. as phagophores and double membrane autophagosomes⁴². We did observe unclosed phagophores in hexa mATG8-KO cells by EM and SR microscopy although we did not detect by enumeration significant accumulation of early autophagic structures. These variances suggest that VPS37A is essential for closure whereas mATG8s act as facilitators augmenting this process, akin to what has been reported for other kinetic effects of mATG8s⁸⁸. In addition, mATG8s are necessary to maintain autophagic organelles in a sealed state, and in the absence of mATG8s the quality of the initial seal or the continuing membrane integrity are compromised. Other studies have shown that in the absence of the mATG8 lipidation machinery, which affects both LC3s and GABARAPs, there is accumulation of unclosed autophagosomes^{81, 89-91}. More recent elegant studies have used autophagy receptors in protease protection assays as a probe for autophagosomal closure indicating that autophagic phagophores can close even in the absence of mATG8s⁶⁹ or mATG8s' lipidation⁷⁰. These variances have been in part explained by the kinetic effects of mATG8s due to longer or shorter times of autophagy induction in different studies, which may be of relevance in our experiments. However, our principal finding that autophagosomal membranes are actively maintained in a sealed state least they become permeant to small molecules such as the MIL probe, indicate that even when morphologically appearing as closed, autophagosomal organelles can be permeant to small molecules in the absence of mATG8s, ATG9A or ESCRTs.

The autophagic structures observed in our study in the absence of key ATG8s, are arrested at a stage morphologically equal to amphisomes, compatible with the ultrastructural images of autophagic profiles previously seen in mATG8s KO cells⁶⁹. In both our work and in the study by Nguyen et al., 69, such organelles do not progress to autolysosomes, indicating a deficiency preventing their conversion into strong lytic compartments. We show here that in mATG8 KO cells (hexa, GABATKO or LC3TKO) amphisomes along with other LC3B⁺ membranes are permeant to small molecules, possibly due to pores remaining in the absence of efficient ESCRT-dependent closure or due to a continuing need to maintain membrane integrity via ESCRTs given that autophagosomes sequester cargo that may potentially and repeatedly compromise delimiting membranes.

The role of both LC3 and GABARAP subsets in maintaining the LC3B membranes in a sealed state is unexpected given that prior observations have indicated that GABARAPs are more important in progression to autolysosomes than LC3s⁴⁹. Since GABARAP appears to be stronger than LC3A in binding to VPS37A, this too can be the consequence of kinetic/facilitator effects of mATG8s. GABARAPs have stronger effects than LC3s in mitophagy assays, albeit LC3A does show some activity⁶⁹.

The role of ATG9A in mammalian cells is under intense study, including its function in autophagosome formation. According to recent reports, its action as lipid scramblase helps in autophagosomal expansion but does not prohibit closure³⁴. Of note, mammalian ATG9A is morphologically seen in non-autophagic vesicles only dynamically making contacts with autophagosomes and is not stably integrated into mammalian autophagosomal membranes⁹². Recent studies support transient ATG9A colocalization with LC3B membranes that can be enhanced by blocking ATG9A recycling^{93, 94}. Regardless of whether ATG9A acts in cis within or in trans upon the autophagic membranes, since it binds mATG8s⁹⁵ it can either way accomplish its role in keeping autophagic membranes sealed via ESCRTs, specifically via the ATG9A-IQGAP1-CHIMP2A triad. Our finding that the scramblase mutant, ATG9A-M33³⁴, does not affect the contribution of ATG9A to sealing of the autophagosomes fits observations by others³⁴. It is also in keeping with the absence of effects of the M33 mutation on other ATG9A-ESCRT-dependent processes such as the repair of plasma membrane⁸⁰.

The apparent porousness of autophagic membranes may be due to a single closure point, a patchwork of membranes that require multiple points of closure, or due to the previously unappreciated aspect that membranes once closed need to be maintained in a sealed state due to the stress of continuous membrane expansion or incidental cargo penetration. Our findings uncover a new function for mATG8s and ATG9A bringing autophagosomal membranes into and maintaining them in a sealed state. Since autophagy plays a role in many diseases and normal physiology, understanding these specific processes is of fundamental and applied significance^{9, 10}.

All references which have been disclosed are incorporated by reference where relevant. The invention is further described in the following claims.

### REFERENCES FIRST SET

1. Mizushima, N., Levine, B., Cuervo, A. M. & Klionsky, D. J. Autophagy fights disease through cellular self-digestion. Nature 451, 1069-1075 (2008).
2. Dong, S. et al. Chaperone-mediated autophagy sustains haematopoietic stem-cell function. Nature 591, 117-123 (2021).
3. Deretic, V. & Lazarou, M. A guide to membrane atg8ylation and autophagy with reflections on immunity. J Cell Biol 221 (2022).
4. Morishita, H. & Mizushima, N. Diverse Cellular Roles of Autophagy. Annu Rev Cell Dev Biol 35, 453-475 (2019).
5. Levine, B. & Kroemer, G. Biological Functions of Autophagy Genes: A Disease Perspective. Cell 176, 11-42 (2019).
6. Lahiri, V., Hawkins, W. D. & Klionsky, D. J. Watch What You (Self-) Eat: Autophagic Mechanisms that Modulate Metabolism. Cell Metab 29, 803-826 (2019).
7. Deretic, V. & Kroemer, G. Autophagy in metabolism and quality control: opposing, complementary or interlinked functions. Autophagy IN press, 1-10 (2021).
8. Deretic, V. Autophagy in inflammation, infection, and immunometabolism. Immunity 54, 437-453 (2021).
9. Mizushima, N. & Levine, B. Autophagy in Human Diseases. N Engl J Med 383, 1564-1576 (2020).
10. Klionsky, D. J. et al. Autophagy in major human diseases. EMBO J 40, e108863 (2021).
11. Mizushima, N., Yoshimori, T. & Ohsumi, Y. The role of Atg proteins in autophagosome formation. Annu Rev Cell Dev Biol 27, 107-132 (2011).
12. Hayashi-Nishino, M. et al. A subdomain of the endoplasmic reticulum forms a cradle for autophagosome formation. Nat Cell Biol 11, 1433-1437 (2009).
13. Tooze, S. A. & Yoshimori, T. The origin of the autophagosomal membrane. Nat Cell Biol 12, 831-835 (2010).
14. Axe, E. L. et al. Autophagosome formation from membrane compartments enriched in phosphatidylinositol 3-phosphate and dynamically connected to the endoplasmic reticulum. J Cell Biol 182, 685-701 (2008).
15. Hara, T. et al. FIP200, a ULK-interacting protein, is required for autophagosome formation in mammalian cells. J Cell Biol 181, 497-510 (2008).
16. Itakura, E. & Mizushima, N. Characterization of autophagosome formation site by a hierarchical analysis of mammalian Atg proteins. Autophagy 6 (2010).
17. Itakura, E. & Mizushima, N. p62 Targeting to the autophagosome formation site requires self-oligomerization but not LC3 binding. The Journal of cell biology 192, 17-27 (2011).
18. Nishimura, T. et al. Autophagosome formation is initiated at phosphatidylinositol synthase-enriched ER subdomains. EMBO J 36, 1719-1735 (2017).
19. Ravikumar, B., Moreau, K., Jahreiss, L., Puri, C. & Rubinsztein, D. C. Plasma membrane contributes to the formation of pre-autophagosomal structures. Nat Cell Biol 12, 747-757 (2010).
20. Moreau, K., Ravikumar, B., Renna, M., Puri, C. & Rubinsztein, D. C. Autophagosome precursor maturation requires homotypic fusion. Cell 146, 303-317 (2011).
21. Soreng, K. et al. SNX18 regulates ATG9A trafficking from recycling endosomes by recruiting Dynamin-2. EMBO Rep (2018).
22. Puri, C. et al. The RAB11A-Positive Compartment Is a Primary Platform for Autophagosome Assembly Mediated by WIPI2 Recognition of PI3P-RAB11A. Dev Cell 45, 114-131 e118 (2018).
23. Longatti, A. et al. TBC1D14 regulates autophagosome formation via Rab11- and ULK1-positive recycling endosomes. J Cell Biol 197, 659-675 (2012).
24. Knaevelsrud, H. et al. Membrane remodeling by the PX-BAR protein SNX18 promotes autophagosome formation. J Cell Biol 202, 331-349 (2013).
25. Puri, C., Renna, M., Bento, C. F., Moreau, K. & Rubinsztein, D. C. Diverse autophagosome membrane sources coalesce in recycling endosomes. Cell 154, 1285-1299 (2013).
26. Ge, L., Melville, D., Zhang, M. & Schekman, R. The ER-Golgi intermediate compartment is a key membrane source for the LC3 lipidation step of autophagosome biogenesis. Elife 2, e00947 (2013).
27. Ge, L., Zhang, M. & Schekman, R. Phosphatidylinositol 3-kinase and COPII generate LC3 lipidation vesicles from the ER-Golgi intermediate compartment. Elife 3, e04135 (2014).
28. Ge, L. et al. Remodeling of ER-exit sites initiates a membrane supply pathway for autophagosome biogenesis. EMBO Rep 18, 1586-1603 (2017).
29. Kumar, S. et al. Phosphorylation of Syntaxin 17 by TBK1 Controls Autophagy Initiation. Dev Cell (2019).
30. Kumar, S. et al. Mammalian hybrid pre-autophagosomal structure HyPAS generates autophagosomes. Cell 184, 5950-5969 e5922 (2021).
31. Kabeya, Y. et al. LC3, a mammalian homologue of yeast Apg8p, is localized in autophagosome membranes after processing. Embo J 19, 5720-5728 (2000).
32. Valverde, D. P. et al. ATG2 transports lipids to promote autophagosome biogenesis. J Cell Biol 218, 1787-1798 (2019).
33. Matoba, K. et al. Atg9 is a lipid scramblase that mediates autophagosomal membrane expansion. Nat Struct Mol Biol 27, 1185-1193 (2020).
34. Maeda, S. et al. Structure, lipid scrambling activity and role in autophagosome formation of ATG9A. Nat Struct Mol Biol 27, 1194-1201 (2020).
35. Ghanbarpour, A., Valverde, D. P., Melia, T. J. & Reinisch, K. M. A model for a partnership of lipid transfer proteins and scramblases in membrane expansion and organelle biogenesis. Proc Natl Acad Sci USA 118 (2021).
36. Zhao, Y. G. & Zhang, H. Autophagosome maturation: An epic journey from the ER to lysosomes. J Cell Biol 218, 757-770 (2019).
37. Gordon, P. B. & Seglen, P. O. Prelysosomal convergence of autophagic and endocytic pathways. Biochem Biophys Res Commun 151, 40-47 (1988).
38. Berg, T. O., Fengsrud, M., Stromhaug, P. E., Berg, T. & Seglen, P. O. Isolation and characterization of rat liver amphisomes. Evidence for fusion of autophagosomes with both early and late endosomes. J Biol Chem 273, 21883-21892 (1998).
39. Eskelinen, E. L. et al. Role of LAMP-2 in lysosome biogenesis and autophagy. Mol Biol Cell 13, 3355-3368 (2002).
40. Liou, W., Geuze, H. J., Geelen, M. J. & Slot, J. W. The autophagic and endocytic pathways converge at the nascent autophagic vacuoles. J Cell Biol 136, 61-70 (1997).
41. Takahashi, Y. et al. An autophagy assay reveals the ESCRT-III component CHMP2A as a regulator of phagophore closure. Nat Commun 9, 2855 (2018).
42. Takahashi, Y. et al. VPS37A directs ESCRT recruitment for phagophore closure. J Cell Biol 218, 3336-3354 (2019).
43. Knorr, R. L., Lipowsky, R. & Dimova, R. Autophagosome closure requires membrane scission. Autophagy 11, 2134-2137 (2015).
44. Zhen, Y. et al. ESCRT-mediated phagophore sealing during mitophagy. Autophagy 16, 826-841 (2020).
45. Flower, T. G. et al. A helical assembly of human ESCRT-I scaffolds reverse-topology membrane scission. Nat Struct Mol Biol 27, 570-580 (2020).
46. Mizushima, N. The ATG conjugation systems in autophagy. Curr Opin Cell Biol 63, 1-10 (2020).
47. Xin, Y. et al. Cloning, expression patterns, and chromosome localization of three human and two mouse homologues of GABA (A) receptor-associated protein. Genomics 74, 408-413 (2001).
48. He, H. et al. Post-translational modifications of three members of the human MAPILC3 family and detection of a novel type of modification for MAPILC3B. J Biol Chem 278, 29278-29287 (2003).
49. Weidberg, H. et al. LC3 and GATE-16/GABARAP subfamilies are both essential yet act differently in autophagosome biogenesis. The EMBO journal 29, 1792-1802 (2010).
50. Ichimura, Y. et al. A ubiquitin-like system mediates protein lipidation. Nature 408, 488-492 (2000).
51. Mizushima, N. et al. A protein conjugation system essential for autophagy. Nature 395, 395-398 (1998).
52. Sanjuan, M. A. et al. Toll-like receptor signalling in macrophages links the autophagy pathway to phagocytosis. Nature 450, 1253-1257 (2007).
53. Galluzzi, L. & Green, D. R. Autophagy-Independent Functions of the Autophagy Machinery. Cell 177, 1682-1699 (2019).
54. Ulferts, R. et al. Subtractive CRISPR screen identifies the ATG16L1/vacuolar ATPase axis as required for non-canonical LC3 lipidation. Cell Rep 37, 109899 (2021).
55. Lee, C., Lamech, L., Johns, E. & Overholtzer, M. Selective Lysosome Membrane Turnover Is Induced by Nutrient Starvation. Dev Cell 55, 289-297 e284 (2020).
56. Loi, M., Raimondi, A., Morone, D. & Molinari, M. ESCRT-III-driven piecemeal micro-ER-phagy remodels the ER during recovery from ER stress. Nat Commun 10, 5058 (2019).
57. Guo, H. et al. Atg5 Disassociates the VIVO-ATPase to Promote Exosome Production and Tumor Metastasis Independent of Canonical Macroautophagy. Dev Cell 43, 716-730 e717 (2017).
58. Leidal, A. M. et al. The LC3-conjugation machinery specifies the loading of RNA-binding proteins into extracellular vesicles. Nat Cell Biol 22, 187-199 (2020).
59. Nakamura, S. et al. LC3 lipidation is essential for TFEB activation during the lysosomal damage response to kidney injury. Nat Cell Biol 22, 1252-1263 (2020).
60. Kumar, S. et al. Mammalian Atg8 proteins and the autophagy factor IRGM control mTOR and TFEB at a regulatory node critical for responses to pathogens. Nat Cell Biol 22, 973-985 (2020).
61. Goodwin, J. M. et al. GABARAP sequesters the FLCN-FNIP tumor suppressor complex to couple autophagy with lysosomal biogenesis. Sci Adv 7, eabj2485 (2021).
62. Kumar, S., Jia, J. & Deretic, V. Atg8ylation as a general membrane stress and remodeling response. Cell Stress 5, 128-142 (2021).
63. Stolz, A., Ernst, A. & Dikic, I. Cargo recognition and trafficking in selective autophagy. Nat Cell Biol 16, 495-501 (2014).
64. Randow, F. & Youle, R. J. Self and nonself: how autophagy targets mitochondria and bacteria. Cell Host Microbe 15, 403-411 (2014).
65. Lamark, T. & Johansen, T. Mechanisms of Selective Autophagy. Annu Rev Cell Dev Biol 37, 143-169 (2021).
66. Weidberg, H. et al. LC3 and GATE-16 N termini mediate membrane fusion processes required for autophagosome biogenesis. Developmental cell 20, 444-454 (2011).
67. Maruyama, T. et al. Membrane perturbation by lipidated Atg8 underlies autophagosome biogenesis. Nat Struct Mol Biol 28, 583-593 (2021).
68. Nguyen, T. N. et al. ATG4 family proteins drive phagophore growth independently of the LC3/GABARAP lipidation system. Molecular Cell 81, 2013-2030. e2019 (2021).
69. Nguyen, T. N. et al. Atg8 family LC3/GABARAP proteins are crucial for autophagosome-lysosome fusion but not autophagosome formation during PINK1/Parkin mitophagy and starvation. J Cell Biol 215, 857-874 (2016).
70. Ohnstad, A. E. et al. Receptor-mediated clustering of FIP200 bypasses the role of LC3 lipidation in autophagy. EMBO J 39, e104948 (2020).
71. England, C. G., Luo, H. & Cai, W. HaloTag technology: a versatile platform for biomedical applications. Bioconjug Chem 26, 975-986 (2015).
72. Bache, K. G. et al. The growth-regulatory protein HCRP1/hVps37A is a subunit of mammalian ESCRT-I and mediates receptor down-regulation. Mol Biol Cell 15, 4337-4346 (2004).
73. Pornillos, O. et al. Structure and functional interactions of the Tsg101 UEV domain. Embo J 21, 2397-2406 (2002).
74. Rogov, V. V. et al. Structural and functional analysis of the GABARAP interaction motif (GIM). EMBO Rep 18, 1382-1396 (2017).
75. Jia, J. et al. Galectins Control mTOR in Response to Endomembrane Damage. Mol Cell 70, 120-135 e128 (2018).
76. Gremke, N. et al. mTOR-mediated cancer drug resistance suppresses autophagy and generates a druggable metabolic vulnerability. Nat Commun 11, 4684 (2020).
77. Gomez-Sanchez, R. et al. Atg9 establishes Atg2-dependent contact sites between the endoplasmic reticulum and phagophores. J Cell Biol 217, 2743-2763 (2018).
78. Sawa-Makarska, J. et al. Reconstitution of autophagosome nucleation defines Atg9 vesicles as seeds for membrane formation. Science 369 (2020).
79. Campisi, D. et al. The core autophagy protein ATG9A controls dynamics of cell protrusions and directed migration. J Cell Biol 221 (2022).
80. Claude-Taupin, A. et al. ATG9A protects the plasma membrane from programmed and incidental permeabilization. Nat Cell Biol 23, 846-858 (2021).
81. Itakura, E., Kishi-Itakura, C., Koyama-Honda, I. & Mizushima, N. Structures containing Atg9A and the ULK1 complex independently target depolarized mitochondria at initial stages of Parkin-mediated mitophagy. Journal of cell science 125, 1488-1499 (2012).
82. Narendra, D., Tanaka, A., Suen, D. F. & Youle, R. J. Parkin is recruited selectively to impaired mitochondria and promotes their autophagy. J Cell Biol 183, 795-803 (2008).

83. McEwan, D. G. et al. PLEKHMI regulates autophagosome-lysosome fusion through HOPS complex and LC3/GABARAP proteins. Mol Cell 57, 39-54 (2015).
84. Hasegawa, J. et al. Autophagosome-lysosome fusion in neurons requires INPP5E, a protein associated with Joubert syndrome. EMBO J 35, 1853-1867 (2016).
85. Shoemaker, C. J. et al. CRISPR screening using an expanded toolkit of autophagy reporters identifies TMEM41B as a novel autophagy factor. PLOS Biol 17, e2007044 (2019).
86. Hedman, A. C., Smith, J. M. & Sacks, D. B. The biology of IQGAP proteins: beyond the cytoskeleton. EMBO Rep 16, 427-446 (2015).
87. Morita, E. et al. Human ESCRT and ALIX proteins interact with proteins of the midbody and function in cytokinesis. EMBO J 26, 4215-4227 (2007).
88. Tsuboyama, K. et al. The ATG conjugation systems are important for degradation of the inner autophagosomal membrane. Science 354, 1036-1041 (2016).
89. Komatsu, M. et al. Impairment of starvation-induced and constitutive autophagy in Atg7-deficient mice. J Cell Biol 169, 425-434 (2005).
90. Fujita, N. et al. An Atg4B mutant hampers the lipidation of LC3 paralogues and causes defects in autophagosome closure. Mol Biol Cell 19, 4651-4659 (2008).
91. Sou, Y. S. et al. The Atg8 conjugation system is indispensable for proper development of autophagic isolation membranes in mice. Mol Biol Cell 19, 4762-4775 (2008).
92. Orsi, A. et al. Dynamic and transient interactions of Atg9 with autophagosomes, but not membrane integration, are required for autophagy. Mol Biol Cell 23, 1860-1873 (2012).
93. Ravussin, A., Brech, A., Tooze, S. A. & Stenmark, H. The phosphatidylinositol 3-phosphate-binding protein SNX4 controls ATG9A recycling and autophagy. J Cell Sci 134 (2021).
94. Zhou, C. et al. Recycling of autophagosomal components from autolysosomes by the recycler complex. Nat Cell Biol 24, 497-512 (2022).
95. Zhang, T., Guo, L. & Yang, Y. Mammalian ATG9s drive the autophagosome formation by binding to LC3. bioRxiv.

### REFERENCES SECOND SET

1. Tanaka, A. et al. Proteasome and p97 mediate mitophagy and degradation of mitofusins induced by Parkin. The Journal of cell biology 191, 1367-1380 (2010).
2. Nguyen, T. N. et al. Atg8 family LC3/GABARAP proteins are crucial for autophagosome-lysosome fusion but not autophagosome formation during PINK1/Parkin mitophagy and starvation. J Cell Biol 215, 857-874 (2016).
3. Claude-Taupin, A. et al. ATG9A protects the plasma membrane from programmed and incidental permeabilization. Nat Cell Biol 23, 846-858 (2021).
4. Kumar, S. et al. Mechanism of Stx 17 recruitment to autophagosomes via IRGM and mammalian Atg8 proteins. J Cell Biol 217, 997-1013 (2018).
5. Kumar, S. et al. Phosphorylation of Syntaxin 17 by TBK1 Controls Autophagy Initiation. Dev Cell (2019).
6. Gu, Y. et al. Mammalian Atg8 proteins regulate lysosome and autolysosome biogenesis through SNAREs. EMBO J 38, e101994 (2019).
7. Moreau, K., Ravikumar, B., Renna, M., Puri, C. & Rubinsztein, D. C. Autophagosome precursor maturation requires homotypic fusion. Cell 146, 303-317 (2011).
8. Matsui, T. et al. Autophagosomal YKT6 is required for fusion with lysosomes independently of syntaxin 17. J Cell Biol (2018).
9. Kumar, S. et al. Mammalian hybrid pre-autophagosomal structure HyPAS generates autophagosomes. Cell 184, 5950-5969 e5922 (2021).
10. UniProt, C. The Universal Protein Resource (UniProt) in 2010. Nucleic Acids Res 38, D142-148 (2010).
11. Roney, J. P. & Ovchinnikov, S. State-of-the-Art Estimation of Protein Model Accuracy using AlphaFold. bioRxiv (2022).
12. Grant, B. J., Rodrigues, A. P., ElSawy, K. M., McCammon, J. A. & Caves, L. S. Bio3d: an R package for the comparative analysis of protein structures. Bioinformatics 22, 2695-2696 (2006).
13. Skjærven, L., Yao, X.-Q., Scarabelli, G. & Grant, B. J. Integrating protein structural dynamics and evolutionary analysis with Bio3D. BMC bioinformatics 15, 1-11 (2014).
14. Eskelinen, E. L. Fine structure of the autophagosome. Methods Mol Biol 445, 11-28 (2008).
15. Smith, C. S., Joseph, N., Rieger, B. & Lidke, K. A. Fast, single-molecule localization that achieves theoretically minimum uncertainty. Nat Methods 7, 373-375 (2010).
16. Huang, F., Schwartz, S. L., Byars, J. M. & Lidke, K. A. Simultaneous multiple-emitter fitting for single molecule super-resolution imaging. Biomed Opt Express 2, 1377-1393 (2011).
17. Wester, M. J. et al. Robust, fiducial-free drift correction for super-resolution imaging. Sci Rep 11, 23672 (2021).
18. Chambers, M. C. et al. A cross-platform toolkit for mass spectrometry and proteomics. Nat Biotechnol 30, 918-920 (2012).
19. Kall, L., Canterbury, J. D., Weston, J., Noble, W. S. & MacCoss, M. J. Semi-supervised learning for peptide identification from shotgun proteomics datasets. Nat Methods 4, 923-925 (2007).
20. Kall, L., Storey, J. D. & Noble, W. S. Non-parametric estimation of posterior error probabilities associated with peptides identified by tandem mass spectrometry. Bioinformatics 24, i42-48 (2008).
21. Kall, L., Storey, J. D., MacCoss, M. J. & Noble, W. S. Assigning significance to peptides identified by tandem mass spectrometry using decoy databases. J Proteome Res 7, 29-34 (2008).

SEQUENCES

SEQ ID NO: 1
YGRKKRRQRRRGGMSWLFP
SEQ ID NO: 2
UGGCAGUGUCUUAGCUGGUUGU
SEQ ID NO: 3
CAAUCAGCAAGUAUACUGCCCU
SEQ ID NO: 4
YGRKKRRQRRRGGWEGQLQDLVLDEY

1. A method of treating an autophagy mediated disease state and/or condition in a patient or subject in need, the method comprising administering to said patient an effective amount of a modulator of ATG8.

2. The method according to claim 1 wherein said autophagy mediated disease state and/or condition is selected from the group consisting of a neurodegenerative disease, an infectious disease, an autoimmune disease, an inflammatory disease and cancer.

3. The method according to claim 1 wherein autophagy mediated disease state and/or condition is cancer, rheumatoid arthritis, malaria, antiphospholipid antibody syndrome, lupus, antiphospholipid antibody syndrome, chronic urticaria and Sjogren's disease.

4. The method according to claim 1 wherein said autophagy mediated disease state and/or condition is cancer.

5. The method according to claim 1 wherein said modulator of ATG8 is an inhibitor of ATG8.

6. The method according to claim 5 wherein said inhibitor is a peptide inhibitor of ATG8 according to SEQ ID NO:1.

7. The method according to claim 6 wherein said peptide inhibitor is combined with at least one compound selected from the group consisting of oleuropein, oleuropein aglycone), hsa-miR-34a, has-miR-34a-5p (SEQ ID NO:2), has-miR-34a-3p (SEQ ID NO: 3), AT110 inhibitor and an Atg9A inhibitory peptide according to SEQ ID NO:4 in order to enhance the effect of the Atg8 inhibitory peptide in treating autophagy mediated disease states and/or conditions.

8. The method according to claim 6 wherein said peptide inhibitor is combined with an inhibitory peptide according to SEQ ID NO:4.

9. The method according to claim 1 wherein said treatment further includes administering an additional autophagy modulator to said patient.

10. The method according to claim 9 wherein said autophagy mediated disease state and/or condition is cancer and said additional autophagy modulator is tetrachlorisophthalonitrile, phenylmercuric acetate, JQ1, 2-methoxyestradiol, 3-methyladenine (3MA), epigallocatechin gallate (EGCG), 3BDO, 5-aminolevulinic acid, 5-azacytidine, 6-thioguanine, A-317491, A-867744, ABT-737, ABT-751, aceglutamide, acetazolamide, afatinib, capsaicin, actigenin, ascorbic acid, curcumin, resveratrol, SP600125, U0126, Bafiliomycin A1, chloroquine, LY294002, SB202190, SB203580, SC79, autophinib, wortmannin, crocin, harmines, mangiferin, tetrachlorisophthalonitrile, cycloheximide, hydroxychloroquine, Lys05, leupeptin, E64d, pepstatin A, or a pharmaceutically acceptable salt thereof.

11. The method according claim 1 wherein said disease state and/or condition is cancer and said patient is further administered an additional anticancer agent.

12. The method according to claim 11 wherein said additional anticancer agent is selected from the group consisting of antimetabolites, inhibitors of topoisomerase I and II, alkylating agents, microtubule inhibitors, tyrosine kinase inhibitors, EGF kinase inhibitors and ABL kinase inhibitors.

13. The method according to claim 1 wherein said cancer is a carcinoma or a tumor.

14. The method according to claim 13 wherein said cancer is a carcinoma or a tumor of the central nervous system.

15. The method according to claim 1 wherein said cancer is pancreatic cancer, a glioma, glioblastoma or a neuroblastoma.

16. A pharmaceutical composition comprising an effective amount of a peptide according to SEQ ID NO:1.

17. The composition according to claim 16 further comprising an effective amount of at least one compound selected from the group consisting of oleuropein, oleuropein aglycone, hsa-miR-34a, has-miR-34a-5p, has-miR-34a-3p or AT110 inhibitor.

18. The composition according to claim 16 further comprising at least one additional autophagy modulator.

19. The composition according to claim 18 wherein said additional autophagy modulator is an inhibitor of autophagy.

20. The composition according to claim 18 wherein said additional autophagy modulator is tetrachlorisophthalonitrile, phenylmercuric acetate, JQ1, 2-methoxyestradiol, 3-methyladenine (3MA), epigallocatechin gallate (EGCG), 3BDO, 5-aminolevulinic acid, 5-azacytidine, 6-thioguanine, A-317491, A-867744, ABT-737, ABT-751, aceglutamide, acetazolamide, afatinib, capsaicin, actigenin, ascorbic acid, curcumin, resveratrol, SP600125, U0126, Bafiliomycin A1, chloroquine, LY294002, SB202190, SB203580, SC79, autophinib, wortmannin, crocin, harmines, mangiferin, tetrachlorisophthalonitrile, cycloheximide, hydroxychloroquine, Lys05, leupeptin, E64d, pepstatin A, or a pharmaceutically acceptable salt thereof.


SEQUENCE LISTING (4 sequences)

SEQ ID NO: 1 - protein, 19 aa, unidentified
YGRKKRRQRRRGGMSWLFP

SEQ ID NO: 2 - RNA, 22 nt, unidentified
  source 1..22: mol_type = other RNA
tggcagtgtcttagctggttgt

SEQ ID NO: 3 - RNA, 22 nt, unidentified
  source 1..22: mol_type = other RNA
caatcagcaagtatactgccct

SEQ ID NO: 4 - protein, 26 aa, unidentified
YGRKKRRQRRRGGWEGQLQDLVLDEY